(19) United States
(12) Patent Application Publication
(10) Pub. No.: US 2026/0317387 A1
(43) Pub. Date: Oct. 15, 2026

(54) MYOSTATIN AND ACTIVIN A IMMUNOGENS AND METHODS OF USE

(71) Applicants: Vaxxinity, Inc., Merritt Island, FL (US); United Biomedical, Inc., Dallas, TX (US)

(72) Inventors: Jean-Cosme Dodart, Merritt Island, FL (US); Cyrille Vivian Gineste, Merritt Island, FL (US); Brett Anthony Thibodeaux, Palm Bay, FL (US); Shuang Ding, Syosset, NY (US); Feng Lin, Roslyn, NY (US)

(21) Appl. No.: 19/384,483

(22) Filed: Nov. 10, 2025

Related U.S. Application Data
(60) Provisional application No. 63/718,433, filed on Nov. 8, 2024.

(51) Int. Cl. C07K 14/475; A61K 39/00; A61K 39/39; A61P 37/04; C07K 16/22

(52) U.S. Cl. CPC C07K 14/475; A61K 39/0005; A61K 39/39; A61P 37/04; C07K 16/22; A61K 2039/55505; A61K 2039/55561; C07K 2319/40

(57) ABSTRACT

This disclosure provides myostatin and activin A peptide immunogen constructs, compositions and methods for their use in, for example, maintaining and improving muscle physiology and/or performance (e.g., increasing muscle mass, strength, function, tone or endurance), preventing and/or treating muscular or musculoskeletal injury, preventing and/or treating disorders associated with muscle loss, lack of muscle strength, insufficient muscle growth, muscle wasting, sarcopenia, or cachexia.

### CROSS-REFERENCE TO RELATED APPLICATIONS

This application claims the benefit of U.S. Provisional Application No. 63/718,433, filed on Nov. 8, 2024, the entire contents of which are incorporated by reference herein in their entirety.

### SEQUENCE LISTING

This application contains a Sequence Listing which has been submitted electronically in XML format. The Sequence Listing XML is incorporated by reference herein. Said XML file, created on Oct. 31, 2025, is named AXE-00301.xml and is 401,105 bytes in size.

### FIELD OF THE DISCLOSURE

This disclosure relates to myostatin and activin A peptide immunogen constructs, compositions and methods of their use (e.g., for maintaining and improving muscle physiology (e.g., increasing muscle mass, strength, tone or endurance), preventing and treating muscular or musculoskeletal injury, preventing and treating a disorder associated with muscle loss or insufficient muscle growth, and/or preventing and treating muscle wasting diseases).

### BACKGROUND

Muscle wasting is a loss of muscle mass characterized by muscles weakening and shrinking. It can occur due to a number of different causes including, for example, age, genetics, disuse, malnutrition, injury, and certain medical conditions affecting the musculoskeletal or nervous systems. Muscle wasting can cause mobility issues, pain, prolong bed rest, injury, and discomfort, which in turn may lead to substantially reduced quality of life.

Sarcopenia is a skeletal muscle disorder characterized by the progressive loss of skeletal muscle mass, strength, and physical performance that occurs with aging (see Sayer et al., 2024, Nat Rev Dis Primers 10(1), doi:10.1038/s41572-024-00550-w; Cruz-Jentoft et al., 2019, The Lancet 393 (10191): 2636-2646). It affects approximately 5-13% of individuals aged 60-70 and increases to 11-50% in those over 80 years old, leading to frailty, falls, and disability (see von Haehling et al., 2010, J Cachexia Sarcopenia Muscle 1(1):1-5; Janssen et al., 2002, J Am Geriatr Soc 50(5):889-896; Baumgartner et al., 1998, Epidemiology of Sarcopenia among the elderly in New Mexico, Vol. 147, https://academic.oup.com/aje/article/147/8/755/88959; von Haehling et al., 2010, J Cachexia Sarcopenia Muscle 1(2):129-133).

There are no FDA-approved drugs for sarcopenia (see Kwak et al., 2019, Ann Geriatr Med Res 23(3):98-104). Although myostatin inhibition has been attempted for muscle growth (see Jang et al., 2021, Nutrients 13(5), doi:10.3390/nu13051508; Haidet et al., 2008, PNAS, www.pnas.orgcgidoi10.1073pnas.0709144105; Sharp et al., 2014, J Int Soc Sports Nutr. 11(1), doi:10.1186/1550-2783-11-S1-P42), most of these strategies had failed to achieve the desired impact in clinical trials (see Rybalka et al., 2020, Cells 9(12), doi:10.3390/cells9122657; Lee et al., 2023, Journals of Gerontology 78:32-37, doi:10.1093/gerona/glad033). In the context of sarcopenia, this failure is attributed to the acute nature of these interventions as well as reduced levels of growth hormone in older adults, impacting muscle growth.

There is a need for new approaches for the prevention and treatment of muscle wasting. There is also a need for new approaches for the prevention and treatment of sarcopenia.

### SUMMARY

In a first aspect, the invention provides a peptide immunogen construct comprising a myostatin B cell epitope, a heterologous T cell (Th) epitope, and an optional heterologous spacer, wherein the B cell epitope is covalently linked to the Th epitope directly or through the optional heterologous spacer.

In some embodiments, the myostatin B cell epitope comprises about 7 to about 30 contiguous amino acids from myostatin or a variant thereof.

In some embodiments, the myostatin B cell epitope comprises about 7 to about 30 contiguous amino acids of SEQ ID NO: 1 or SEQ ID NO: 2 or a variant thereof. In some embodiments, the myostatin B cell epitope comprises up to 3, up to 4, up to 5, or up to 6, amino acid substitutions as compared to the corresponding sequence of the 7-30 amino acid fragment of SEQ ID NO: 1 or SEQ ID NO: 2.

In some embodiments, the myostatin B cell epitope comprises a variant of a 7-30 amino acid fragment of SEQ ID NO: 1 or SEQ ID NO: 2, and the variant comprises 1, 2, 3, 4, 5, 6, or 7 amino acid substitutions, deletions, or insertions as compared to the corresponding sequence of the 7-30 amino acid fragment of SEQ ID NO: 1 or SEQ ID NO: 2.

In some embodiments, the myostatin B cell epitope comprises a variant of a 7-30 amino acid fragment of SEQ ID NO: 1 or SEQ ID NO: 2, and the variant comprises 1, 2, or 3 amino acid substitutions as compared to the corresponding sequence of the 7-30 amino acid fragment of SEQ ID NO: 1 or SEQ ID NO: 2.

In some embodiments, the myostatin B cell epitope comprises one or more amino acid substitutions as compared to the sequence of SEQ ID NO: 1 or SEQ ID NO: 2 to facilitate cyclization of the sequence comprising the myostatin B cell epitope.

In some embodiments, the myostatin B cell epitope comprises 1, 2, or 3 amino acid substitutions as compared to the sequence of SEQ ID NO: 1 or SEQ ID NO: 2 to facilitate cyclization of the sequence comprising the myostatin B cell epitope.

In some embodiments, the myostatin B cell epitope is cyclic or cyclized. In some embodiments, the modifications allowing for cyclization described herein were made to make the B cell epitope structure resemble the tertiary structure of the naturally occurring protein fragment.

In some embodiments, the invention provides a peptide immunogen construct comprising a myostatin B cell epitope, a heterologous T cell (Th) epitope, and an optional heterologous spacer, wherein the B cell epitope is covalently linked to the Th epitope directly or through the optional heterologous spacer; wherein the myostatin B cell epitope comprises about 7 to about 30 contiguous amino acids from myostatin, or a variant thereof, and/or wherein the myostatin B cell epitope is cyclized.

In some embodiments, the myostatin B cell epitope comprises or consists of an amino acid sequence within loop 1, loop 2, loop 3, or loop 4 of myostatin, or a variant thereof. In some embodiments, the myostatin B cell epitope comprises or consists of the amino acid sequence of any one of SEQ ID NOs: 3-10, 95-109, 145-164, and 166 or a variant thereof. In some embodiments, the myostatin B cell epitope comprises or consists of the amino acid sequence of SEQ ID NO: 5, 97, 98, 101, 105, 146, 147, 151-155, 157, 159, 161, 163, 164, or 166, or a variant thereof. In some embodiments, the myostatin B cell epitope comprises or consists of the amino acid sequence of SEQ ID NO: 5, 97, 98, 101, or 105, or a variant thereof. In some embodiments, the myostatin B cell epitope comprises or consists of the amino acid sequence of SEQ ID NO: 5. In some embodiments, the myostatin B cell epitope comprises or consists of the amino acid sequence of SEQ ID NO: 97. In some embodiments, the myostatin B cell epitope comprises or consists of the amino acid sequence of SEQ ID NO: 98. In some embodiments, the myostatin B cell epitope comprises or consists of the amino acid sequence of SEQ ID NO: 101. In some embodiments, the myostatin B cell epitope comprises or consists of the amino acid sequence of SEQ ID NO: 105. In some embodiments, the myostatin B cell epitope comprises or consists of the amino acid sequence of SEQ ID NO: 146, 147, 151, 152, 154, 155, 157, 159, 161, 163, 164, 166, or a variant thereof. In some embodiments, the myostatin B cell epitope comprises or consists of the amino acid sequence of SEQ ID NO: 146. In some embodiments, the myostatin B cell epitope comprises or consists of the amino acid sequence of SEQ ID NO: 147. In some embodiments, the myostatin B cell epitope comprises or consists of the amino acid sequence of SEQ ID NO: 151, or a variant thereof. In some embodiments, the myostatin B cell epitope comprises or consists of the amino acid sequence of SEQ ID NO: 152, or a variant thereof. In some embodiments, the myostatin B cell epitope comprises or consists of the amino acid sequence of SEQ ID NO: 153, or a variant thereof. In some embodiments, the myostatin B cell epitope comprises or consists of the amino acid sequence of SEQ ID NO: 154, or a variant thereof. In some embodiments, the myostatin B cell epitope comprises or consists of the amino acid sequence of SEQ ID NO: 155, or a variant thereof. In some embodiments, the myostatin B cell epitope comprises or consists of the amino acid sequence of SEQ ID NO: 157, or a variant thereof. In some embodiments, the myostatin B cell epitope comprises or consists of the amino acid sequence of SEQ ID NO: 159, or a variant thereof. In some embodiments, the myostatin B cell epitope comprises or consists of the amino acid sequence of SEQ ID NO: 161, or a variant thereof. In some embodiments, the myostatin B cell epitope comprises or consists of the amino acid sequence of SEQ ID NO: 163, or a variant thereof. In some embodiments, the myostatin B cell epitope comprises or consists of the amino acid sequence of SEQ ID NO: 164, or a variant thereof. In some embodiments, the myostatin B cell epitope comprises or consists of the amino acid sequence of SEQ ID NO: 166, or a variant thereof.

In some embodiments, the myostatin B cell epitope peptide is acetylated on the N-terminus. In some embodiments, the myostatin B cell epitope peptide comprises an amide group on the C-terminus. In some embodiments, the myostatin B cell epitope peptide is acetylated on the N-terminus and comprises an amide group on the C-terminus. In some embodiments, the myostatin B cell epitope peptide is cyclized using the N-terminus amino group and not acetylated on the N-terminus.

In some embodiments, the myostatin B cell epitope peptide is cyclized (or cyclic). The myostatin B cell epitope peptide can be cyclized using any bridge, linkage or methodology known in the art or described herein. In some embodiments, the myostatin B cell epitope peptide is cyclic due to the presence of amino acids naturally occurring in the myostatin B cell epitope peptide fragment. In some embodiments, the myostatin B cell epitope peptide is cyclic due to the presence of one or more (e.g., two) amino acid substitutions relative to the naturally occurring amino acid sequence of the myostatin B cell epitope peptide fragment. In some embodiments, the myostatin B cell epitope peptides are cyclic due to, e.g., the presence of cysteine residues, whether naturally occurring in the sequences or inserted, e.g., by substitution. In some embodiments, the myostatin B cell epitope peptide comprises, or is cyclized with, a disulfide (S—S) linkage. In some embodiments, the myostatin B cell epitope peptide comprises, or is cyclized with, a lactam linkage.

In some embodiments, the Th epitope is derived from a pathogenic protein. In some embodiments, the Th epitope comprises or consists of a sequence selected from any one of SEQ ID NOs: 19-47 or a variant thereof.

In some embodiments, the Th epitope comprises or consists of the sequence of SEQ ID NO: 30, 32, or 33. In some embodiments, the Th epitope comprises or consists of the sequence of SEQ ID NO: 30. In some embodiments, the Th epitope comprises or consists of the sequence of SEQ ID NO: 32. In some embodiments, the Th epitope comprises or consists of the sequence of SEQ ID NO: 33.

In some embodiments, the myostatin B cell epitope and the Th epitope are linked to one another by direct covalent linkage. In other embodiments, the myostatin B cell epitope and the Th epitope are linked to one another by the heterologous spacer.

In some embodiments, the heterologous spacer comprises or consists of one or more amino acids, which are optionally selected from: Lys-, Gly-, Lys-Lys-Lys-, (α, ε-N)Lys, Lys-Lys-Lys-εLys (SEQ ID NO: 192), ε-N-Lys-Lys-Lys-Lys (SEQ ID NO: 91), and Pro-Pro-Xaa-Pro-Xaa-Pro, wherein Xaa is any amino acid or is aspartic acid (SEQ ID NO: 90). In some embodiments, the heterologous spacer comprises or consists of Lys-Lys-Lys-εLys (SEQ ID NO: 192). In some embodiments, the heterologous spacer comprises or consists of ε-N-Lys-Lys-Lys-Lys (SEQ ID NO: 91). In some embodiments, the heterologous spacer comprises or consists of (α, ε-N)Lys.

In some embodiments, (a) the myostatin B cell epitope is located N-terminal to the Th epitope; (b) the Th epitope is located N-terminal to the myostatin B cell epitope; (c) the myostatin B cell epitope is flanked with a Th epitope on both N-terminal and C-terminal ends; (d) any of (a)-(c) wherein the myostatin B cell epitope(s) is linked to the Th epitope(s) by direct covalent linkage; or (e) any one of (a)-(c) wherein the myostatin B cell epitope(s) is linked to the Th epitope(s) by the heterologous spacer. In some embodiments, the myostatin B cell epitope is located N-terminal to the Th epitope. In some embodiments, the Th epitope is located N-terminal to the myostatin B cell epitope.

In some embodiments, the peptide immunogen construct is of following formula: (Th)ₘ-(A)ₙ-(myostatin B cell epitope), or (myostatin B cell epitope)-(A)ₙ-(Th)ₘ, or (Th)ₘ-(A)ₙ-(myostatin B cell epitope)-(A)ₙ-(Th)ₘ, wherein Th is the T helper epitope: A is the heterologous spacer, which optionally is present and optionally comprises an amino acid; m is from 1 to about 4; and n is from 0 to about 10, wherein optionally the C-terminus of the peptide immunogen construct comprises α-COOH or α-CONH₂ of an amino acid. In some embodiments, the peptide immunogen construct is of following formula: (Th)ₘ-(A)ₙ-(myostatin B cell epitope), wherein Th is the T helper epitope; A is the heterologous spacer, which optionally is present and optionally comprises an amino acid; m is from 1 to about 4; and n is from 0 to about 10, and wherein the C-terminus of the peptide immunogen construct comprises α-COOH or α-CONH₂ of an amino acid. In some embodiments, the peptide immunogen construct is of following formula: (myostatin B cell epitope)-(A)ₙ-(Th)ₘ, wherein Th is the T helper epitope; A is the heterologous spacer, which optionally is present and optionally comprises an amino acid; m is from 1 to about 4; and n is from 0 to about 10, and wherein the C-terminus of the peptide immunogen construct comprises α-COOH or α-CONH₂ of an amino acid. In some embodiments of the formulas provided herein, m is 1 and n is 1. In some embodiments, more than one myostatin B cell epitope is present in the peptide immunogen constructs described herein, e.g., 1-4 myostatin B cell epitopes. In some embodiments, two or three myostatin B cell epitopes are present in the peptide immunogen constructs described herein. In other embodiments, one (1) myostatin B cell epitope is present in the peptide immunogen constructs described herein.

In some embodiments, the peptide immunogen construct comprises or consists of the sequence of any one of SEQ ID NOs: 48-71, 120-134, 167-186 and 188 or a variant thereof. In some embodiments, the peptide immunogen construct comprises or consists of the sequence of SEQ ID NO: 54, 55, 56, 122, 123, 126, 130, 168, 169, 173, 174, 175, 176, 177, 178, 179, 181, 183, 185, or 186, or a variant thereof. In some embodiments, the peptide immunogen construct comprises or consists of the sequence of SEQ ID NO: 54, 55, 56, 122, 123, 126 or 130, or a variant thereof. In some embodiments, the peptide immunogen construct comprises or consists of the sequence of SEQ ID NO: 54, 55, or 56. In some embodiments, the peptide immunogen construct comprises or consists of the sequence of SEQ ID NO: 54. In some embodiments, the peptide immunogen construct comprises or consists of the sequence of SEQ ID NO: 55. In some embodiments, the peptide immunogen construct comprises or consists of the sequence of SEQ ID NO: 56. In some embodiments, the peptide immunogen construct comprises or consists of the sequence of SEQ ID NO: 122. In some embodiments, the peptide immunogen construct comprises or consists of the sequence of SEQ ID NO: 123. In some embodiments, the peptide immunogen construct comprises or consists of the sequence of SEQ ID NO: 126. In some embodiments, the peptide immunogen construct comprises or consists of the sequence of SEQ ID NO: 130. In some embodiments, the peptide immunogen construct comprises or consists of the sequence of SEQ ID NO: 168, 169, 173, 174, 175, 176, 177, 178, 179, 181, 183, 185, 186 or a variant thereof. In some embodiments, the peptide immunogen construct comprises or consists of the sequence of SEQ ID NO: 168. In some embodiments, the peptide immunogen construct comprises or consists of the sequence of SEQ ID NO: 169. In some embodiments, the peptide immunogen construct comprises or consists of the sequence of SEQ ID NO: 173. In some embodiments, the peptide immunogen construct comprises or consists of the sequence of SEQ ID NO: 174. In some embodiments, the peptide immunogen construct comprises or consists of the sequence of SEQ ID NO: 176. In some embodiments, the peptide immunogen construct comprises or consists of the sequence of SEQ ID NO: 177. In some embodiments, the peptide immunogen construct comprises or consists of the sequence of SEQ ID NO: 179. In some embodiments, the peptide immunogen construct comprises or consists of the sequence of SEQ ID NO: 181. In some embodiments, the peptide immunogen construct comprises or consists of the sequence of SEQ ID NO: 183. In some embodiments, the peptide immunogen construct comprises or consists of the sequence of SEQ ID NO: 185. In some embodiments, the peptide immunogen construct comprises or consists of the sequence of SEQ ID NO: 186.

In another aspect, the invention provides a peptide immunogen construct comprising an activin A B cell epitope, a heterologous T cell (Th) epitope, and an optional heterologous spacer, wherein the B cell epitope is covalently linked to the Th epitope directly or through the optional heterologous spacer.

In some embodiments, the activin A B cell epitope comprises about 7 to about 30 contiguous amino acids from activin A or a variant thereof.

In some embodiments, the activin A B cell epitope comprises about 7 to about 30 contiguous amino acids of SEQ ID NO: 12 or a variant thereof. In some embodiments, the activin A B cell epitope comprises up to 3, up to 4, up to 5, or up to 6, amino acid substitutions as compared to the corresponding sequence of the 7-30 amino acid fragment of SEQ ID NO: 12.

In some embodiments, the activin A B cell epitope comprises a variant of a 7-30 amino acid fragment of SEQ ID NO: 12, and the variant comprises 1, 2, 3, 4, 5, 6, or 7 amino acid substitutions, deletions, or insertions as compared to the corresponding sequence of the 7-30 amino acid fragment of SEQ ID NO: 12.

In some embodiments, the activin A B cell epitope comprises a variant of a 7-30 amino acid fragment of SEQ ID NO: 12, and the variant comprises 1, 2, or 3 amino acid substitutions as compared to the corresponding sequence of the 7-30 amino acid fragment of SEQ ID NO: 12.

In some embodiments, the activin A B cell epitope comprises one or more amino acid substitutions as compared to the sequence of SEQ ID NO: 12 to facilitate cyclization of the sequence comprising the activin A B cell epitope.

In some embodiments, the activin A B cell epitope comprises 1, 2, or 3 amino acid substitutions as compared to the sequence of SEQ ID NO: 12 to facilitate cyclization of the sequence comprising the activin A B cell epitope.

In some embodiments, the activin A B cell epitope is cyclic or cyclized.

In some embodiments, the invention provides a peptide immunogen construct comprising an activin A B cell epitope, a heterologous T cell (Th) epitope, and an optional heterologous spacer, wherein the B cell epitope is covalently linked to the Th epitope directly or through the optional heterologous spacer; wherein the activin A B cell epitope comprises about 7 to about 30 contiguous amino acids from myostatin, or a variant thereof, and/or wherein the activin A B cell epitope is cyclized.

In some embodiments, the activin A B cell epitope comprises or consists of an amino acid sequence within loop 1, loop 2, loop 3, or loop 4 of activin A, or a variant thereof. In some embodiments, the activin A B cell epitope comprises or consists of the amino acid sequence of any one of SEQ ID NOs: 13-18, 110-119, 187, and 189 or a variant thereof. In some embodiments, the activin A B cell epitope comprises or consists of the amino acid sequence of SEQ ID NO: 14-18, 111, 116, 118, 119, 187, or a variant thereof. In some embodiments, the activin A B cell epitope comprises or consists of the amino acid sequence of SEQ ID NO: 14, 15, 17, 116, 119, 187, or a variant thereof. In some embodiments, the activin A B cell epitope comprises or consists of the amino acid sequence of SEQ ID NO: 111, 116, 118, 119, or 187 or a variant thereof. In some embodiments, the activin A B cell epitope comprises or consists of the amino acid sequence of SEQ ID NO: 111. In some embodiments, the activin A B cell epitope comprises or consists of the amino acid sequence of SEQ ID NO: 116. In some embodiments, the activin A B cell epitope comprises or consists of the amino acid sequence of SEQ ID NO: 118. In some embodiments, the activin A B cell epitope comprises or consists of the amino acid sequence of SEQ ID NO: 119. In some embodiments, the activin A B cell epitope comprises or consists of the amino acid sequence of SEQ ID NO: 187. In some embodiments, the activin A B cell epitope comprises or consists of the amino acid sequence of SEQ ID NO: 14. In some embodiments, the activin A B cell epitope comprises or consists of the amino acid sequence of SEQ ID NO: 15. In some embodiments, the activin A B cell epitope comprises or consists of the amino acid sequence of SEQ ID NO: 16. In some embodiments, the activin A B cell epitope comprises or consists of the amino acid sequence of SEQ ID NO: 17. In some embodiments, the activin A B cell epitope comprises or consists of the amino acid sequence of SEQ ID NO: 18.

In some embodiments, the activin A B cell epitope peptide is acetylated on the N-terminus. In some embodiments, the activin A B cell epitope peptide comprises an amide group on the C-terminus. In some embodiments, the activin A B cell epitope peptide is acetylated on the N-terminus and comprises an amide group on the C-terminus. In some embodiments, the activin A B cell epitope peptide is cyclized using the N-terminus amino group and not acetylated on the N-terminus.

In some embodiments, the activin A B cell epitope peptide is cyclized (or cyclic). The activin A B cell epitope peptide can be cyclized using any bridge, linkage or methodology known in the art or described herein. In some embodiments, the activin A B cell epitope peptide is cyclic due to the presence of amino acids naturally occurring in the activin A B cell epitope peptide fragment. In some embodiments, the activin A B cell epitope peptide is cyclic due to the presence of one or more (e.g., two) amino acid substitutions relative to the naturally occurring amino acid sequence of the activin A B cell epitope peptide fragment. In some embodiments, the activin A B cell epitope peptides are cyclic due to, e.g., the presence of cysteine residues, whether naturally occurring in the sequences or inserted. e.g., by substitution. In some embodiments, the activin A B cell epitope peptide comprises, or is cyclized with, a disulfide (S—S) linkage. In some embodiments, the activin A B cell epitope peptide comprises, or is cyclized with, a lactam linkage.

In some embodiments, the Th epitope is derived from a pathogenic protein. In some embodiments, the Th epitope comprises or consists of a sequence selected from any one of SEQ ID NOs: 19-47 or a variant thereof.

In some embodiments, the Th epitope comprises or consists of the sequence of SEQ ID NO: 30, 32, or 33. In some embodiments, the Th epitope comprises or consists of the sequence of SEQ ID NO: 30. In some embodiments, the Th epitope comprises or consists of the sequence of SEQ ID NO: 32. In some embodiments, the Th epitope comprises or consists of the sequence of SEQ ID NO: 33.

In some embodiments, the myostatin B cell epitope and the Th epitope are linked to one another by direct covalent linkage.

In some embodiments, the activin A B cell epitope and the Th epitope are linked to one another by the heterologous spacer.

In some embodiments, the heterologous spacer comprises or consists of one or more amino acids, which are optionally selected from: Lys-, Gly-, Lys-Lys-Lys-, (α, ε-N)Lys, Lys-Lys-Lys-εLys (SEQ ID NO: 192), ε-N-Lys-Lys-Lys-Lys (SEQ ID NO: 91), and Pro-Pro-Xaa-Pro-Xaa-Pro, wherein Xaa is any amino acid or is aspartic acid (SEQ ID NO: 90). In some embodiments, the heterologous spacer comprises or consists of Lys-Lys-Lys-εLys (SEQ ID NO: 192). In some embodiments, the heterologous spacer comprises or consists of ε-N-Lys-Lys-Lys-Lys (SEQ ID NO: 91). In some embodiments, the heterologous spacer comprises or consists of (α, ε-N)Lys.

In some embodiments, (a) the activin A B cell epitope is located N-terminal to the Th epitope; (b) the Th epitope is located N-terminal to the activin A B cell epitope; (c) the activin A B cell epitope is flanked with a Th epitope on both N-terminal and C-terminal ends; (d) any of (a)-(c) wherein the activin A B cell epitope(s) is linked to the Th epitope(s) by direct covalent linkage; or (e) any one of (a)-(c) wherein the activin A B cell epitope(s) is linked to the Th epitope(s) by the heterologous spacer. In some embodiments, the activin A B cell epitope is located N-terminal to the Th epitope. In some embodiments, the Th epitope is located N-terminal to the activin A B cell epitope.

In some embodiments, the peptide immunogen construct is of following formula: (Th)ₘ-(A)ₙ-(activin A B cell epitope) or (activin A B cell epitope)-(A)ₙ-(Th)ₘ or (Th)ₘ-(A)ₙ-(activin A B cell epitope)-(A)ₙ-(Th)ₘ, wherein Th is the T helper epitope; A is the heterologous spacer, which optionally is present and optionally comprises an amino acid; m is from 1 to about 4; and n is from 0 to about 10, wherein optionally the C-terminus of the peptide immunogen construct comprises α-COOH or α-CONH₂ of an amino acid. In some embodiments, the peptide immunogen construct is of following formula: (Th)ₘ-(A)ₙ-(activin A B cell epitope), wherein Th is the T helper epitope; A is the heterologous spacer, which optionally is present and optionally comprises an amino acid; m is from 1 to about 4; and n is from 0 to about 10, and wherein the C-terminus of the peptide immunogen construct comprises α-COOH or α-CONH₂ of an amino acid. In some embodiments, the peptide immunogen construct is of following formula: (activin A B cell epitope)-(A)ₙ-(Th)ₘ, wherein Th is the T helper epitope; A is the heterologous spacer, which optionally is present and optionally comprises an amino acid; m is from 1 to about 4; and n is from 0 to about 10, and wherein the C-terminus of the peptide immunogen construct comprises α-COOH or α-CONH₂ of an amino acid. In some embodiments of the formulas provided herein, m is 1 and n is 1. In some embodiments, more than one activin A B cell epitope is present in the peptide immunogen constructs described herein, e.g., 1-4 activin A B cell epitopes. In some embodiments, two or three activin A B cell epitopes are present in the peptide immunogen constructs described herein. In other embodiments, one (1) activin A B cell epitope is present in the peptide immunogen constructs described herein.

In some embodiments, the peptide immunogen construct comprises or consists of the sequence of any one of SEQ ID NOs: 72-89, 135-144, and 190-191 or a variant thereof. In some embodiments, the peptide immunogen construct comprises or consists of the sequence of SEQ ID NO: 75, 76, 78, 79, 85, 136, 138, 139, 140, 141, 142, 143, 144, or 190, or a variant thereof. In some embodiments, the peptide immunogen construct comprises or consists of the sequence of SEQ ID NO: 75, 78, 79, 85, 141, 143, 144, or 190, or a variant thereof. In some embodiments, the peptide immunogen construct comprises or consists of the sequence of SEQ ID NO: 136, 141, 143, 144, or 190, or a variant thereof. In some embodiments, the peptide immunogen construct comprises or consists of the sequence of SEQ ID NO: 136. In some embodiments, the peptide immunogen construct comprises or consists of the sequence of SEQ ID NO: 141. In some embodiments, the peptide immunogen construct comprises or consists of the sequence of SEQ ID NO: 143. In some embodiments, the peptide immunogen construct comprises or consists of the sequence of SEQ ID NO: 144. In some embodiments, the peptide immunogen construct comprises or consists of the sequence of SEQ ID NO: 190. In some embodiments, the peptide immunogen construct comprises or consists of the sequence of SEQ ID NO: 75. In some embodiments, the peptide immunogen construct comprises or consists of the sequence of SEQ ID NO: 76. In some embodiments, the peptide immunogen construct comprises or consists of the sequence of SEQ ID NO: 78. In some embodiments, the peptide immunogen construct comprises or consists of the sequence of SEQ ID NO: 79. In some embodiments, the peptide immunogen construct comprises or consists of the sequence of SEQ ID NO: 84. In some embodiments, the peptide immunogen construct comprises or consists of the sequence of SEQ ID NO: 85.

In another aspect, the invention provides a combination peptide immunogen construct comprising two or more B cell epitopes, a heterologous T cell (Th) epitope, an optional heterologous linker, and an optional heterologous spacer, wherein the two or more B cell epitopes are covalently linked to the Th epitope directly or through the optional heterologous linker and/or optional heterologous spacer. In some embodiments, the two or more B cell epitopes are one to four (1-4) B cell epitopes. In some embodiments, the two or more B cell epitopes are two (2) or three (3) B cell epitopes. In some embodiments, the two or more B cell epitopes are two (2) B cell epitopes. In some embodiments, the two or more B cell epitopes are two or more (e.g., two) identical B cell epitopes, such as any B cell epitope described herein. In some embodiments, the two or more B cell epitopes are two or more (e.g., two) non-identical, different B cell epitopes, such as any two or more B cell epitopes described herein. In some embodiments, the two or more B cell epitopes are two or more (e.g., two) myostatin B cell epitopes (e.g., any myostatin B cell epitopes described herein, identical or different). In some embodiments, the two or more B cell epitopes are two or more (e.g., two) activin A B cell epitopes (e.g., any activin A B cell epitopes described herein, identical or different). In some embodiments, the two or more (e.g., two) B cell epitopes comprise at least one myostatin B cell epitope (e.g., any myostatin B cell epitope described herein) and at least one activin A B cell epitope (e.g., any activin A B cell epitope described herein). In some embodiments, the Th epitope is any Th epitope described herein. In some embodiments, the heterologous spacer is any heterologous spacer described herein. In some embodiments, the heterologous linker is any linker described herein. In some embodiments, the heterologous linker comprises one or more amino acids (e.g., one or more glycines). In some embodiments, the heterologous linker is an amino acid peg derivative. In some embodiments, the heterologous linker is AeeA (i.e., 8-amino-3,6-dioxaoctanoic acid or 2-[2-(2-aminoethoxy)ethoxy]acetic acid (CAS:134978-97-5)). In other embodiments, other heterologous linkers can be used, for example, and without limitation, a linker comprising 3 to 6 pegs (e.g., 1-Amino-3,6,9,12,15,18-hexaoxahenicosan-21-oic acid (6pe), 1-amino-4,7,10,13-tetraoxapentadecanoic acid (3peg)), 6-aminohexanoic acid, or one or more epsilon-lysine. In some embodiments, the two or more B cell epitopes can be positioned in linear and/or sequential manner. In some embodiments, the two or more B cell epitopes are positioned in a non-linear and/or non-sequential manner. In some embodiments, the two or more B cell epitopes are branched off from a Th epitope, e.g., as described herein.

In some embodiments, provided herein is a combination myostatin—activin A peptide immunogen construct comprising a myostatin B cell epitope (e.g., any myostatin B cell epitope described herein), an activin A B cell epitope (e.g., any activin A B cell epitope described herein), and a T helper cell epitope (such as any T helper cell epitope described herein). In some embodiments, the B cell epitopes and the Th epitope are linked by a heterologous spacer (such as any heterologous spacer described herein). In some embodiments, the Th epitope comprises or consists of the sequence of SEQ ID NO: 30, 32, or 33. In some embodiments, the heterologous spacer comprises or consists of Lys-Lys-Lys-εLys (SEQ ID NO: 192), ε-N-Lys-Lys-Lys-Lys (SEQ ID NO: 91), or (α, ε-N)Lys. In some embodiments of the combination myostatin—activin A peptide immunogen construct, the myostatin B cell epitope comprises or consists of the amino acid sequence of any one of SEQ ID NOs: 3-10, 95-109, 145-164, and 166 or a variant thereof. In some embodiments, the myostatin B cell epitope comprises or consists of the amino acid sequence of SEQ ID NO: 5, 97, 146, 147, 152-155, 157, 159, 161, 163, or 166. In some embodiments, the myostatin B cell epitope is of SEQ ID NO: 97. In some embodiments of the combination myostatin—activin A peptide immunogen construct, the activin A B cell epitope comprises or consists of the amino acid sequence of any one of SEQ ID NOs: 13-18, 110-119, 187, and 189 or a variant thereof. In some embodiments, the activin A B cell epitope comprises or consists of the amino acid sequence of SEQ ID NO: 14-18, 111, 116, 118, 119, or 187. In some embodiments, the activin A B cell epitope comprises or consists of the amino acid sequence of SEQ ID NO: 14, 15, 17, 116, 119, or 187. In some embodiments, the activin A B cell epitope of SEQ ID NO: 116. In some embodiments, the myostatin B cell epitope is of SEQ ID NO: 97 and the activin A B cell epitope of SEQ ID NO: 116.

In some embodiments, the combination peptide immunogen construct comprises B cell epitopes sequentially positioned in a linear peptide. In other embodiments, the combination peptide immunogen comprises B cell epitopes that are positioned in a non-linear or non-sequential manner or branched off from a Th epitope. In some embodiments, the B cell epitope peptides are attached to the same side (N-terminal or C-terminal) of the Th epitope using a branched amino acid, which can be: (i) a diamino acid if the B-cell epitopes are attached to the N-terminus of a Th epitope, or (ii) a dicarboxylic acid if the B-cell epitopes are attached to the C-terminus of a Th epitope. Any diamino acid and dicarboxylic acid known in the art or described herein can be used. In some embodiments, the diamino acid is lysine. Other diamino acids include, without limitation, ornithine, 2,4-Diaminobutyric acid, and 2,3-diaminopropionic acid. In some embodiments, the dicarboxylic acid is glutamic acid. Other dicarboxylic acids include, without limitation, aspartic acid and a-amino adipic acid. Between the branched amino acid and the B cell epitope peptide, a heterologous linker can be used. In some embodiments, the heterologous linker comprises one or more amino acids (e.g., one or more Glycines). In some embodiments, the heterologous linker is an amino acid peg derivative. In some embodiments, the heterologous linker is AeeA (i.e., 8-amino-3,6-dioxaoctanoic acid or 2-[2-(2-aminoethoxy)ethoxy]acetic acid (CAS:134978-97-5)). In other embodiments, other heterologous linkers can be used, for example, and without limitation, a linker comprising 3 to 6 pegs (e.g., 1-Amino-3,6,9,12,15,18-hexaoxahenicosan-21-oic acid (6pe), 1-amino-4,7,10,13-tetraoxapentadecanoic acid (3peg)), 6-aminohexanoic acid, or one or more epsilon-lysine. The combination peptide immunogen construct described herein can comprise the B cell epitope peptides described herein covalently linked to any Th epitope using any heterologous spacer described herein or known in the art, wherein the heterologous spacer connects the branched amino acid to the Th epitope. In some embodiments, the heterologous spacer comprises an amino acid (e.g., 2, 3, 4 or 5 Lysine residues). In some embodiments, the heterologous spacer comprises or consists of Lys-Lys-Lys-εLys (SEQ ID NO: 192), ε-N-Lys-Lys-Lys-Lys (SEQ ID NO: 91), or (α, ε-N)Lys. In some embodiments, the combination myostatin—activin A peptide immunogen construct is acetylated on its terminal NH2.

In some embodiments, the combination myostatin—activin A peptide immunogen construct is of SEQ ID NO: 165 (i.e., Ac-STVINHYRMRGHSPFANLKSSAeeA(Ac-QKYPHTHLVHQANPRGSAGPAeeA)K-KKKrKUbith1 (SEQ ID NO: 165), where the peptide between ( ) indicates that it is off the side chain of the Lys and not the alpha amino group, AeeA is a peg spacer, and the Ac indicates that the construct is acetylated on its terminal NH2). In other embodiments, another myostatin B cell epitope (e.g., any one described herein), another activin A B cell epitope (e.g., any one described herein), another linker (e.g., any one described herein), and/or another T helper cell epitope (e.g., any one described herein) can be used in the combination myostatin-activin peptide immunogen constructs of the invention (instead of those used in the construct of SEQ ID NO: 165).

The invention further provides a nucleic acid molecule encoding a peptide immunogen construct described herein.

The invention further provides a vector comprising a nucleic acid molecule described herein.

The invention further provides an isolated antibody or an epitope-binding fragment thereof that specifically binds to the myostatin B cell epitope of the peptide immunogen construct or an activin A B cell epitope of a peptide immunogen construct described herein.

The invention further provides a composition comprising one or more of: (a) a peptide immunogen construct described herein, a nucleic acid described herein, a vector described herein, an isolated antibody described herein, or a peptide immunogen construct described herein and a peptide immunogen construct described herein, and (b) a pharmaceutically acceptable carrier, diluent, or adjuvant.

The invention further provides a composition comprising: (a) a peptide immunogen construct comprising a myostatin B cell epitope, a heterologous T cell (Th) epitope, and an optional heterologous spacer, wherein the B cell epitope is covalently linked to the Th epitope directly or through the optional heterologous spacer; optionally wherein the myostatin B cell epitope comprises about 7 to about 30 contiguous amino acids from myostatin, or a variant thereof, and optionally wherein the myostatin B cell epitope is cyclized, (b) a peptide immunogen construct comprising an activin A B cell epitope, a heterologous T cell (Th) epitope, and an optional heterologous spacer, wherein the B cell epitope is covalently linked to the Th epitope directly or through the optional heterologous spacer; optionally wherein the activin A B cell epitope comprises about 7 to about 30 contiguous amino acids from myostatin, or a variant thereof, and optionally wherein the activin A B cell epitope is cyclized, and (c) a pharmaceutically acceptable carrier, diluent, or adjuvant.

In some embodiments, the composition comprises an adjuvant that is a mineral salt of aluminum, which optionally is selected from Al(OH)₃ and AlPO₄.

In some embodiments, the composition comprises a CpG oligonucleotide (e.g., CpG1).

In some embodiments, administration of the peptide immunogen constructs comprising a myostatin B cell epitope described herein stimulate generation of one or more antibodies to myostatin, wherein such one or more antibodies inhibit myostatin-induced signaling, e.g., in a cell-based assay.

In some embodiments, administration of the peptide immunogen constructs comprising an activin A B cell epitope described herein stimulate generation of one or more antibodies to activin A, wherein such one or more antibodies inhibit activin A-induced signaling, e.g., in a cell-based assay.

The invention further provides a method of maintaining or improving muscle physiology in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of maintaining or improving muscle mass or growth in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of maintaining or improving muscle strength in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of maintaining or improving muscle tone in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of maintaining or improving muscle endurance in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of maintaining or improving muscle function or performance in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of preventing or treating muscular injury in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of preventing or treating musculoskeletal injury in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of preventing and treating a disorder associated with muscle loss or insufficient muscle growth in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of treating or preventing the development of muscle wasting in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of treating, preventing, reducing, inhibiting, or slowing the development of one or more symptom of a muscle wasting disease in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof.

The invention further provides a method of treating, preventing, reducing, inhibiting, or slowing age- or disease-related muscle loss in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof.

The invention further provides a method of treating, preventing, reducing, inhibiting, or slowing the development of sarcopenia in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof.

The invention further provides a method of preventing loss or increasing lean body mass (and optionally reducing fat mass) in a subject in need thereof, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof. In some embodiments, the subject has or is at risk of loss of lean body mass (such as muscle wasting). In some embodiments, the subject is obese. In some embodiments, the subject is overweight. In some embodiments, the subject has BMI of at least or over 25 kg/m². In some embodiments, the subject has BMI of at least or over 30 kg/m². In some embodiments, the subject has BMI of at least or over 35 kg/m². In some embodiments, the subject has BMI of at least or over 40 kg/m². In some embodiments, the subject has a metabolic disease, disorder or syndrome. In some embodiments, the subject is suffering from weight-related or obesity-related disease or disorder (e.g., a metabolic disease, disorder or syndrome). In some embodiments, the subject does not have an age-related growth hormone deficit. In some embodiments, the subject is under 60 years of age, under 65 years of age, under 70 years of age, or under 80 years of age.

The invention also provides a method of reducing fat mass in a subject in need thereof, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof. In some embodiments, the subject is obese. In some embodiments, the subject is overweight. In some embodiments, the subject has BMI of at least or over 25 kg/m². In some embodiments, the subject has BMI of at least or over 30 kg/m². In some embodiments, the subject has BMI of at least or over 35 kg/m². In some embodiments, the subject has BMI of at least or over 40 kg/m². In some embodiments, the subject has a metabolic disease, disorder or syndrome. In some embodiments, the subject is suffering from weight-related or obesity-related disease or disorder (e.g., a metabolic disease, disorder or syndrome). In some embodiments, the subject treated to achieve a reduction in fat mass does not have an age-related growth hormone deficit. In some embodiments, the subject treated to achieve a reduction in fat mass is under 60 years of age, under 65 years of age, under 70 years of age, or under 80 years of age.

The invention further provides a method of preventing loss of muscle, or increasing muscle strength and/or performance, in a subject in need thereof, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof. In some embodiments, the subject has sarcopenia or cachexia.

The invention also provides a method of increasing muscle strength and/or performance in a subject at least or over 55, 60, 65, 70, 75 or 80 years of age (or a subject suffering from a reduced level of growth hormone, e.g., relative to an average level in a human subject having a normal level of growth hormone), the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof. In some embodiments, the subject is over 60 years old. In some embodiments, the subject is over 65 years old. In some embodiments, the subject is over 70 years old. In some embodiments, the subject has a reduced muscle strength and/or performance as measured by any test known in the art or described herein (e.g., reduced relative to an average muscle strength and/or performance in a human subject having a normal level of growth hormone). In some embodiments, the subject exhibits muscle wasting. In some embodiments, the subject has sarcopenia or cachexia. In some embodiments, the subject has a reduced muscle mass. In some embodiments, the reduction in muscle strength, muscle performance and/or muscle mass is relative to the same subject at an earlier time period (e.g., 1 year, 2 years, 3 years, or 5 years prior). In some embodiments, the reduction in muscle strength, muscle performance and/or muscle mass is relative to a human subject having an average muscle strength, performance or mass, e.g., at 30, 35, 40, 45 or 50 years of age.

The invention also provides a method of treating or preventing sarcopenia, or improving or ameliorating a symptom thereof, in a subject in need there (such as an age-related sarcopenia), the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof. In some embodiments, the subject is at least or over 55, 60, 65, 70, 75 or 80 years of old. In some embodiments, the subject is over 60 years old. In some embodiments, the subject is over 65 years old. In some embodiments, the subject is over 70 years old. In some embodiments, the subject is suffering from a reduced level of growth hormone, e.g., relative to an average level in a human subject having a normal level of growth hormone.

The invention further provides a method of maintaining or improving bone physiology in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, or a composition described herein, or a combination of any thereof.

The invention further provides a method of inducing an immune response to myostatin and/or activin A in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof.

The invention further provides a method of treating or preventing insulin resistance in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof.

The invention further provides a method of maintaining or improving glucose metabolism in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof.

The invention further provides a method of treating or preventing metabolic disease, disorder or syndrome in a subject, the method comprising administering to the subject an effective amount of an immunotherapy targeting myostatin and/or activin A, wherein the immunotherapy is selected from a peptide immunogen construct described herein, a nucleic acid molecule described herein, a vector described herein, an isolated antibody described herein, a composition described herein, or a combination thereof.

In some embodiments, the subject has or is at risk of developing muscle wasting due to an inherited or genetic cause, inflammation, infection, injury, systemic disease, toxin, or other environmental exposure. In some embodiments, the subject has or is at risk of developing muscle wasting due to immobilization, prolonged bed rest, and/or injury. In some embodiments, the subject has or is at risk of developing muscle wasting due to sarcopenia or progressive loss of muscle strength due to aging.

In some embodiments, the subject has an inherited or genetic myopathy, which is optionally selected from muscular dystrophy (e.g., Duchenne Muscular Dystrophy (DMD) and Becker Muscular Dystrophy (BMD), myotonic dystrophy (e.g., DM1 and DM2), facioscapulohumeral muscular dystrophy, oculopharyngeal muscular dystrophy, and limb girdle muscular dystrophy), congenital myopathy (e.g., nemaline myopathy and central core myopathy), metabolic myopathy (e.g., acid maltase or acid alpha-1, 4-glucosidase deficiency (Pompe's disease), glycogen storage disorders 3-11, carnitine deficiency, fatty acid oxidation defects, and carnitine palmitoyl transferase deficiency), and mitochondrial myopathy.

In some embodiments, the subject has or is at risk of developing an autoimmune or inflammatory myopathy (i.e., myositis), which is optionally selected from dermatomyositis, polymyositis, inclusion body myositis, and juvenile myositis, or another autoimmune disorder (e.g., myasthenia gravis).

In some embodiments, the subject has or is at risk of developing an infection, which optionally is selected from an infection by a virus (e.g., HIV, influenza virus, and Epstein-Barr virus), a bacterium (e.g., pyomyositis, S. aureus, and streptococci), a spirochete (e.g., Lyme disease), and a parasite (e.g., trichinosis).

In some embodiments, the subject has or is at risk of exposure to a toxin, which optionally is selected from a medication (e.g., a cholesterol-lowering medication (e.g., a statin), propofol, amiodaron, colchicine, chloroquine, an antiviral, a protease inhibitor, omeprazole, and tryptophan), alcohol, and toluene.

In some embodiments, the subject has or is at risk of developing a systemic disease, which optionally is selected from an endocrine disorder (e.g., thyroid, parathyroid, pituitary, and adrenal disorder), systemic inflammatory disease (e.g., systemic lupus erythematosus, rheumatoid arthritis, scleroderma, Sjogren's syndrome, mixed connective disease, and sarcoidosis), electrolyte imbalance, critical illness myopathy, and amyloid myopathy.

In some embodiments, the subject has or is at risk of developing a neurological disease or condition, which optionally is selected from amyotrophic lateral sclerosis (ALS), multiple sclerosis (MS), spinal muscular atrophy (SMA), Guillain-Barre syndrome, carpel tunnel syndrome, Charcot-Marie-Tooth disease, Parkinson's disease, Lewy body disease, polio, spinal cord injury, peripheral nerve injury, stroke, and nerve damage caused by diabetes, toxins, or alcohol.

In some embodiments, the subject is or is expected to be present in an environment with decreased or no gravity, e.g., during a space flight or being at a space station or the like.

In some embodiments, the subject has or experiences prolonged inactivity (e.g., is bedridden, has a seated job or a sedentary lifestyle, or cannot move limbs due to stroke or other brain disease), burn, long-term corticosteroid therapy, malnutrition, anorexia, cachexia, osteoarthritis, advanced age, cancer, chronic kidney disease, heart failure, or chronic obstructive pulmonary disease.

In some embodiments, the subject has or is at risk of developing one or more symptom selected from muscle loss, movement issues, balance problems, trouble walking, trouble using arms or legs, contractures, muscle rigidity, muscle twitching, fasciculations, muscle cramps, numbness, tingling, painful sensations, breathing problems, curved spine, heart problems, swallowing problems (which may lead to, e.g., nutritional problems and aspiration pneumonia), reduced muscle mass (e.g., in general or in one limb), weakness (e.g., in general or in one limb), pain, discomfort, one arm or leg being smaller than the other, weakness in one arm or leg, numbness or tingling in an arm or leg, trouble walking, balance problems, facia weakness, gradual memory loss, reduced muscle strength, impaired ability to perform physical activities, decrease in muscle size, cramps, stiffness, spasms, fatigue, motor delay, respiratory impairment, and bulbar muscle dysfunction.

In some embodiments, the subject has or is at risk of developing insulin resistance, type II diabetes, elevated A1C levels, pre-diabetes, metabolic syndrome, hypertension, hyperglycemia, hypercholesterolemia, high triglycerides, overweight, or obesity.

In some embodiments, the subject is a mammal, e.g., a human. In some embodiments, the subject is a female. In some embodiments, the subject is a male.

The invention also includes use of any of the peptide immunogen constructs, nucleic acids, vectors, compositions, and combinations in methods such as any of those described herein, and use of these materials in the preparation of medicaments for carrying out such methods.

Other features and advantages of the invention will be apparent from the following description and the claims.

### BRIEF DESCRIPTION OF THE DRAWINGS

FIG. 1 is a graph showing antibody titers against myostatin resulting from immunization of Guinea pigs with the indicated myostatin peptide immunogen constructs.

FIG. 2 is a graph showing immunogenicity of the peptide immunogen construct of SEQ ID NO: 55 injected into Guinea pigs.

FIG. 3 is a graph showing the results of a neutralization assay testing peptide (SEQ ID NO: 55)-induced antibodies in HEK293 cells.

FIG. 4 is a set of graphs showing the effects of peptide (SEQ ID NO: 55)-induced antibodies on fiber width and number of nuclei in C2C12 cells.

FIG. 5 is a schematic showing a treatment regimen used for in vivo studies.

FIG. 6 is a series of graphs showing immunogenicity at week 11 of mice vaccinated with the indicated doses of peptide immunogen construct of SEQ ID NO: 55.

FIG. 7 is a graph showing body weights of peptide construct (SEQ ID NO: 55)-vaccinated mice at the indicated times.

FIG. 8 is a set of graphs showing twitch tension and tetanic tension in tibialis anterior isolated from peptide construct (SEQ ID NO: 55)-vaccinated mice.

FIG. 9 is a table and a set of graphs showing immunogenicity of the indicated activin A peptide constructs injected into Guinea pigs.

FIG. 10 is a set of graphs showing the results of an assay of the effects of purified activin A IgGs on activin A-induced SMAD2/3 signaling.

FIG. 11 is a graph showing immunogenicity of the indicated activin A peptide constructs in rats at week 6 post-vaccination.

FIG. 12 is a graph showing immunogenicity of the indicated myostatin peptide constructs in rats at week 6 post-vaccination.

FIG. 13 is a graph showing immunogenicity of the indicated myostatin or activin A peptide constructs in Guinea pigs at 15 weeks post-vaccination.

FIG. 14 is a graph showing the change in body weights over time of Guinea pigs immunized with the indicated myostatin or activin A peptide construct.

FIG. 15A is a graph showing the immunogenicity of the indicated latent myostatin peptide constructs in rats at week 15 post-vaccination and FIG. 15B shows antibody titers against latent myostatin resulting from the immunization.

FIG. 16A is a graph showing the immunogenicity of the indicated promyostatin peptide constructs in rats at week 15 post-vaccination and FIG. 16B shows antibody titers against promyostatin resulting from the immunization.

FIG. 17A is a graph showing the immunogenicity of the indicated mature myostatin peptide constructs in rats at week 15 post-vaccination and FIG. 17B shows antibody titers against mature myostatin resulting from the immunization.

FIG. 18 is a graph showing antibody titers against mature myostatin or GDF-11 at 15 weeks after the immunization of rats with the myostatin construct of SEQ ID NO: 168.

FIG. 19A is a graph showing the immunogenicity of the indicated activin A peptide constructs in rats at week 15 post-vaccination and FIG. 19B shows antibody titers against mature activin A resulting from the immunization.

FIG. 20 is a graph showing the immunogenicity of the indicated myostatin peptide constructs in rats at week 15 post-immunization.

FIG. 21 is a graph showing the immunogenicity of the indicated activin A peptide constructs in rats at week 15 post-immunization.

FIG. 22 depicts an exemplary structure of a bivalent construct (containing two B cell epitopes), specifically the construct of SEQ ID NO: 165.

FIGS. 23A-23B show that immunization against myostatin (“MSTN”) and Activin A (“Act A”) using the peptide immunogen constructs of the invention stimulated robust production of anti-MSTN and anti-Act A antibodies. FIG. 23A shows timeline and treatment regimen over 24 weeks. FIG. 23B shows antibody titers as absorbance readings against log dilution at week 15. Peptide immunogen construct V13 (SEQ ID NO: 122) and peptide immunogen construct V35 (SEQ ID NO: 165) treated mice sera were evaluated by MSTN ELISA and peptide immunogen construct V07 (SEQ ID NO: 141) and peptide immunogen construct V35 (SEQ ID NO: 165) treated mice sera were evaluated by Act A ELISA.

FIGS. 24A-24B show forelimb grip strength increases following immunization against MSTN and/or Act A in growth hormone (GH) sufficient littermate control mice (FIG. 24A) and in GH deficient dwarf mice (FIG. 24B) (* indicates p<0.05).

FIGS. 25A-25D show lean and fat mass ratios (as % body weight) in mice following immunization against MSTN and/or Act A. FIG. 25A shows lean mass ratios in GH sufficient littermate control mice. FIG. 25B shows fat mass ratios in GH sufficient littermate control mice. FIG. 25C shows lean mass ratios in GH deficient dwarf mice. FIG. 25D shows fat mass ratios in GH deficient dwarf mice.

FIGS. 26A-26D show transcriptomic changes in the gastrocnemius muscle of the anti-MSTN and/or anti-Act A immunized mice. FIGS. 26A-26B show top 25 upregulated and top 25 downregulated genes in differential expression analysis in the gastrocnemius muscle of GH sufficient littermate control mice (FIG. 26A) and in GH deficient dwarf mice (FIG. 26B). FIGS. 26C-26D show a heatmap of differentially expressed genes in placebo and anti-MSTN transcriptome in GH deficient dwarf mice (FIG. 26C) and a Gene Ontology (GO) enrichment analysis for molecular functions of the differentially expressed genes (FIG. 26D).

FIGS. 27A-27D show forelimb grip strength, and lean and fat mass ratios (as % body weight) in aged mice following immunization against MSTN and/or Act A. FIGS. 27A-27B show forelimb grip strength at week 17 (FIG. 27A) and at week 23 (FIG. 27B). FIG. 27C shows lean mass ratios in mice. FIG. 27D shows fat mass ratios in mice (* indicates p<0.05; ** indicates p<0.01; *** indicates p<0.001).

FIGS. 28A-28F show energy expense and gastrocnemius muscle lipid constituent changes in aged mice following immunization against MSTN and/or Act A. FIGS. 28A-28B show total energy expense over a 45-hour period in aged males (FIG. 28A) and in aged females (FIG. 28B). FIGS. 28C-28D show heatmaps of overall lipid content changes in the gastrocnemius muscle in aged males (FIG. 28C) and in aged females (FIG. 28D). FIGS. 28E-28F show phosphatidylcholine levels (FIG. 28E) and phosphatidylethanolamine levels (FIG. 28F) in aged female gastrocnemius muscle (* indicates p<0.05).

### DETAILED DESCRIPTION

The present disclosure is based, in part, on the discovery that certain myostatin and activin A peptides can be used to induce an immune response to myostatin and activin A, respectively, leading to improvements in muscle physiology and, thus, improvements in certain features of muscle wasting.

Accordingly, the disclosure is directed to certain myostatin and activin A peptides, as well as peptide immunogen constructs and compositions that include these peptides.

The disclosure additionally is directed to methods of maintaining or improving muscle physiology (e.g., maintaining or increasing one, two, three, four or more of: muscle mass, growth, strength, tone, endurance, function or performance) in a subject in need thereof by administration of a peptide immunogen construct or a related molecule or composition described herein. In some embodiments, the disclosure is directed to treating a subject having a muscle strength or performance deficiency, or muscle wasting, by administration of a peptide immunogen construct or composition described herein. In some embodiments, the muscle deficiency treated in accordance with the methods provided herein is a deficiency in muscle strength. In some embodiments, the disclosure is directed to treating, preventing, or promoting recovery from sarcopenia in a subject by administration of a peptide immunogen construct or composition described herein. In some embodiments, the disclosure is directed to treating a subject suffering from age-related (e.g., where the subject is at least 60 or at least 70 years of age) reduction in muscle strength by administration of a peptide immunogen construct or composition described herein. In some embodiments, the disclosure is directed to treating a subject having cachexia by administration of a peptide immunogen construct or composition described herein. In some embodiments, the disclosure is directed to preventing sarcopenia, cachexia or an age-related reduction in muscle strength by administration of a peptide immunogen construct or composition described herein. In some embodiments, the disclosure is directed to treating a subject having a muscle deficiency (such as a deficiency in muscle strength) associated with a reduction in growth hormone (GH) level (such as due to aging) by administration of a peptide immunogen construct or composition described herein. In some embodiments, the subject treated in accordance with the disclosure is 60 years old, 65 years old, 70 years old, or older, and optionally exhibits a reduced GH level (e.g., relative to the level in a subject at an earlier age, e.g., at 30, 40 or 50 years of age). In some embodiments, the subject treated in accordance with the disclosure is 60 years old, 65 years old, 70 years old, or older, and optionally exhibits a reduced insulin-like growth factor 1 (IGF-1) level (e.g., relative to the level in a subject at an earlier age, e.g., at 30, 40 or 50 years of age). In some embodiments, the subject treated (e.g., for sarcopenia or another disorder described herein) exhibits a reduced GH level and/or a reduced IGF-1 level, e.g., due to aging. In some embodiments, the disclosure is directed to improving muscle performance in a subject having a reduced level of growth hormone, e.g., due to aging, by administration of a peptide immunogen construct or composition described herein. In some embodiments, the disclosure is directed to treating sarcopenia in a subject having a reduced level of growth hormone, e.g., due to aging, by administration of a peptide immunogen construct or composition described herein.

The disclosure additionally is directed to methods of preventing or treating a muscle wasting disease or condition in a subject in need thereof by administration of a peptide immunogen construct or a related molecule or composition described herein. The present disclosure is also directed to methods of treating, preventing, reducing, or inhibiting one or more symptoms of a muscle wasting disease. In some embodiments, the disclosure is directed to methods of treating, preventing, reducing, or improving sarcopenia or one or more symptoms of sarcopenia by administration of a peptide immunogen construct or composition described herein. In some embodiments, the disclosure is directed to methods of treating, preventing, reducing, or improving cachexia or one or more symptoms of sarcopenia by administration of a peptide immunogen construct or composition described herein. In some embodiments, the disclosure is directed to methods of treating, preventing, reducing, or improving reduction in muscle strength (e.g., due to any condition or disorder) by administration of a peptide immunogen construct or composition described herein. In some embodiments, the disclosure is directed to methods of treating, preventing, reducing, or improving age-related reduction in muscle strength by administration of a peptide immunogen construct or composition described herein (e.g., where the treated subject is at least or more than 60 years of age, 65 years of age, 70 years of age, 75 years of age, or 80 years of age, optionally wherein the subject is a female). In some embodiments, the treatment effects and improvements described herein can be achieved in a subject having a reduced level of growth hormone, for example due to aging (e.g., a subject at least or more than 60 years of age, 65 years of age, 70 years of age, 75 years of age, or 80 years of age, optionally wherein the subject is a female). In some embodiments, the compositions and methods described herein are effective to increase muscle strength in a subject having a growth hormone deficiency or decreased levels of growth hormone (e.g., due to aging). In some embodiments, the treatment effects and improvements described herein (e.g., increase in muscle strength) can be achieved without increase in muscle mass. For example, in some embodiments, the disclosure is directed to methods of treating, preventing, reducing, or improving sarcopenia in subjects having a decreased level of growth hormone (e.g., without increasing muscle mass), by administration of a peptide immunogen construct or composition described herein. Further, the disclosure is directed to methods of preventing and treating insulin resistance and related diseases and conditions. Advantageously, the compositions and methods of the disclosure can be used to provide substantial benefits for inhibiting, slowing the development of, or treating the diseases and conditions described herein (or one or more symptoms thereof), leading to improved quality of life and prolonged health span.

The compositions and methods of the disclosure are described in an exemplary manner below.

The section headings used herein are for organizational purposes only and are not to be construed as limiting the subject matter described.

Unless otherwise indicated, all technical and scientific terms used herein have the same meaning as commonly understood by those of ordinary skill in the art to which this invention belongs. In case of conflict, the present specification, including explanations of terms, will control. In addition, the materials, methods, and examples are illustrative only and not intended to be limiting.

The singular terms “a,” “an,” and “the” include the respective plural terms, unless context indicates otherwise. Similarly, the word “or” is intended to include “and” unless the context clearly indicates otherwise. Hence “comprising A or B” means including A or B, or A and B. Although methods and materials similar or equivalent to those described herein can be used in the practice or testing of the disclosed methods, suitable methods and materials are described below.

The term “about,” when used to modify a numeric value herein, indicate that deviations of up to 10% above and below the numeric value remain within the intended meaning of the recited value. In some embodiments, the deviation is up to about 7.5%, 6%, 5%, 4%, 3%, 2% or 1% above and below the recited value.

All publications, patent applications, patents, and other references mentioned herein are incorporated by reference herein in their entirety. All references or portions of references cited in this application are expressly incorporated herein by reference in their entirety for any purpose as if each individual publication, patent or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

### Immunotherapeutic Agents and Compositions

The compositions and methods of the disclosure can include or use, for example, one or more peptide or peptide fusion (e.g., a peptide immunogen construct as described herein), a protein (e.g., an antibody), or a nucleic acid molecule (e.g., mRNA or nucleic acid in a viral vector) encoding one of said molecules, wherein the molecule is directed against myostatin or activin A. In some embodiments, the compositions and methods include or employ one or more molecules directed against myostatin and one or more molecules directed against activin A.

### B Cell Epitope Peptides

In some embodiments, the compositions and methods of the disclosure employ a peptide immunogen construct including a B cell epitope from myostatin linked to a heterologous T helper cell (Th) epitope, directly or through an optional heterologous linker or spacer.

The B cell epitope portion of the myostatin peptide immunogen constructs can optionally include about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from mature myostatin, which is located at the C-terminal end of myostatin preproprotein, and corresponds, e.g., to the sequence from about the aspartic acid at amino acid position 267 (D267) to about the serine at amino acid position 375 (S375) of full-length myostatin preproprotein (SEQ ID NO: 1). The sequence of human mature myostatin is set forth in SEQ ID NO: 2. In some embodiments, the B cell epitope portion of the myostatin peptide immunogen constructs can optionally include about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from full-length myostatin preproprotein (SEQ ID NO: 1). In some embodiments, the B cell epitope portion of the myostatin peptide immunogen constructs can optionally include about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from about the leucine at amino acid position 80 (L80) to about the leucine at amino acid position 270 (L270) of full-length myostatin preproprotein (SEQ ID NO: 1). The heterologous Th epitope portion of the peptide immunogen constructs can optionally be derived from pathogenic proteins. The B cell epitope and Th epitope portions of the peptide immunogen constructs act together when administered to a subject to stimulate the generation of antibodies that specifically recognize and bind to the myostatin B cell epitope portion of the constructs.

Accordingly, the phrase “myostatin peptide immunogen construct,” as used herein, refers to a peptide containing (a) a B cell epitope having about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from myostatin (e.g., mature myostatin or myostatin preprotein); (b) a heterologous Th epitope; and (c) an optional heterologous spacer.

In certain embodiments, the myostatin peptide immunogen construct can be represented by the formulae: (Th)ₘ-(A)ₙ-(myostatin peptide)ₚ-X, (myostatin peptide)ₚ-(A)ₙ-(Th)ₘ-X, or (Th)ₘ-(A)ₙ-(myostatin peptide)ₚ-(A)ₙ-(Th)ₘ-X, wherein Tb is a heterologous T helper epitope; A is an optional heterologous spacer; (myostatin peptide) is a myostatin B cell epitope having about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from mature myostatin (e.g., a peptide derived from loop 1, loop 2, loop 3, or loop 4 of mature myostatin, e.g., as described herein); X is an α-COOH or α-CONH₂ of an amino acid; m is an integer from 1 to about 4; p is an integer from 1 to about 4 (e.g., 2); and n is an integer from 0 to about 10. In certain embodiments, the myostatin peptide immunogen construct can be represented by the formulae: (Th)ₘ-(A)ₙ-(myostatin peptide)ₚ-X, (myostatin peptide)ₙ-(A)ₙ-(Th)ₘ-X, or (Th)ₘ-(A)ₙ-(myostatin peptide)ₚ-(A)ₙ-(Th)ₘ-X, wherein Th is a heterologous T helper epitope; A is an optional heterologous spacer; (myostatin peptide) is a myostatin B cell epitope having about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from myostatin preprotein; X is an α-COOH or α-CONH₂ of an amino acid; m is an integer from 1 to about 4; p is an integer from 1 to about 4 (e.g., 2); and n is an integer from 0 to about 10. In some embodiments, A is an amino acid and n indicates the number of amino acids, wherein each A can be identical to one another or one or more of the A's can be a different amino acid. It is to be understood that the myostatin peptides described herein can optionally include one or more modifications (e.g., one or more substitution, deletion, or addition), for example, one or more (e.g., 1, 2, 3, 4, or 5) substitutions, which optionally may be designed to facilitate peptide cyclization. In some embodiments, the peptides are linear. In other embodiments, the peptides are cyclic due to, e.g., the presence of, e.g., cysteine residues, whether naturally occurring in the sequences or inserted, e.g., by substitution. The various components of the disclosed myostatin peptide immunogen constructs are described further below, after a description of activin A peptides.

In some embodiments, the compositions and methods of the disclosure employ a peptide immunogen construct including a B cell epitope from activin A linked to a heterologous T helper cell (Th) epitope, directly or through an optional heterologous linker or spacer.

The B cell epitope portion of the activin A peptide immunogen constructs can optionally include about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from mature activin A, which is located at the C-terminal end of activin A preproprotein. The sequence of human mature activin A is set forth in SEQ ID NO: 12. The heterologous Th epitope portion of the peptide immunogen constructs can optionally be derived from pathogenic proteins. The B cell epitope and Th epitope portions of the peptide immunogen constructs act together when administered to a subject to stimulate the generation of antibodies that specifically recognize and bind to the activin A B cell epitope portion of the constructs.

Accordingly, the phrase “activin A peptide immunogen construct,” as used herein, refers to a peptide containing (a) a B cell epitope having about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from activin A (e.g., mature activin A); (b) a heterologous Th epitope; and (c) an optional heterologous spacer.

In certain embodiments, the peptide immunogen construct can be represented by the formulae: (Th)ₘ-(A)ₙ-(activin A peptide)ₚ-X, (activin A peptide)ₚ-(A)ₙ-(Th)ₘ-X, or (Th)ₘ-(A)ₙ-(activin A peptide)ₚ-(A)ₙ-(Th)ₘ-X, wherein Th is a heterologous T helper epitope; A is an optional heterologous spacer; (activin A peptide) is an activin A B cell epitope having about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues from mature activin A (e.g., a peptide derived from loop 1, loop 2, loop 3, or loop 4 of mature activin A, e.g., as described herein); X is an α-COOH or α-CONH₂ of an amino acid; m is an integer from 1 to about 4; p is an integer from 1 to about 4 (e.g., 2); and n is an integer from 0 to about 10. In some embodiments, A is an amino acid and n indicates the number of amino acids, wherein each A can be identical to one another or one or more of the A's can be a different amino acid. It is to be understood that the activin A peptides described herein can optionally include one or more modifications (e.g., one or more substitution, deletion, or addition), for example, one or more (e.g., 1, 2, 3, 4, or 5) substitutions, which optionally may be designed to facilitate peptide cyclization. In some embodiments, the peptides are linear. In other embodiments, the peptides are cyclic due to, e.g., the presence of, e.g., cysteine residues, whether naturally occurring in the sequences or inserted, e.g., by substitution. The various components of the disclosed activin A peptide immunogen constructs are described further below.

In some embodiments, the B cell epitope peptides described herein are acetylated on the N-terminus (e.g., when the B cell epitope is linked to the N-terminal end of the T helper cell epitope). In some embodiments, the B cell epitope peptides described herein comprise an amide group on the C-terminus (e.g., when the B cell epitope is linked to the C-terminal end of the T helper cell epitope). In some embodiments, the B cell epitope peptide is acetylated on the N-terminus and comprises an amide group on the C-terminus. In some embodiments, the B cell epitope peptide described herein is cyclized using the N-terminus amino group and not acetylated on the N-terminus.

### Myostatin and Activin A Fragments

The term “myostatin preproprotein” and the like, as used herein, refers to (a) the full-length myostatin preproprotein from any organism that expresses myostatin. In some embodiments, the myostatin preproprotein is human. In certain embodiments, the full-length human myostatin preproprotein has 375 amino acids (see, e.g., SEQ ID NO: 1). Myostatin preproprotein includes an N-terminal core of hydrophobic amino acids, which act as a signal sequence for secretion. Proteolytic processing in the Golgi at an RSRR site by a serine protease, such as furin or another member of the proprotein convertase family, generates an N-terminal latency-associated peptide (LAP) and C-terminal mature myostatin.

The term “mature myostatin,” as used herein, refer to the amino acid sequence of the myostatin preproprotein that is C-terminal from the serine protease cleavage site RSRR. In certain embodiments, myostatin or mature myostatin is human. In certain embodiments, mature myostatin has the amino acid sequence between residues 267-375 (SEQ ID NO: 2) of myostatin preproprotein (SEQ ID NO: 1).

The phrase “myostatin fragment,” “myostatin peptide,” or “B cell epitope of myostatin,” as used herein, refers to a portion of the full-length myostatin sequence that includes about 7 to about 30 (e.g., 7-15, 7-14, 7-13, 7-12, 7-11, or 7-10, or 7, 8, 9, 10, 11, 12, 13, 14, or 15) contiguous amino acid residues from the myostatin sequence. The various myostatin fragments described herein can be referred to by their amino acid positions in relation to the full-length sequence of prepromyostatin represented by SEQ ID NO: 1. In some embodiments, a myostatin fragment includes one or more amino acid changes (e.g., substitutions, deletions, or additions), for example, one or more amino acid substitutions (e.g., 1, 2, 3, 4, 5, or 6 amino acid substitutions). In some embodiments, the one or more amino acid changes, for example, the one or more (e.g., 1, 2, 3, 4, or 5) amino acid substitutions, are designed to facilitate cyclization of the myostatin fragment. In some embodiments, the myostatin fragment has the sequence of SEQ ID NO: 3, 4, 5, 6, 7, 8, 9, or 10 (see Table 1). In some embodiments, the myostatin fragment has the sequence of SEQ ID NO: 95, 96, 97, 98, 99, 100, 101, 102, 103, 104, 105, 106, 107, 108, or 109 (see Table 1). In some embodiments, the myostatin fragment has the sequence of SEQ ID NO: 145, 146, 147, 148, 149, 150, 151, 152, 153, or 154 (see Table 1). In some embodiments, the myostatin fragment has the sequence of SEQ ID NO: 155, 156, 157, 158, 159, 160, 161, 162, 163, 164, or 166 (see Table 1).

The term “activin preproprotein” and the like, as used herein, refers to (a) the full-length activin A preproprotein from any organism that expresses activin A. In some embodiments, the activin A preproprotein is human. In certain embodiments, the full-length human activin A preproprotein has 426 amino acids (see, e.g., SEQ ID NO: 11). Activin A preproprotein includes an N-terminal core of hydrophobic amino acids, which act as a signal sequence for secretion. Proteolytic processing in the Golgi at an RRRR site by a serine protease, such as furin or another member of the proprotein convertase family, generates C-terminal mature activin A, which will homo- or hetero-dimerize to form a physiologically active molecule.

The terms “mature activin A,” as used herein, refer to the amino acid sequence of the activin A preproprotein that is C-terminal from the serine protease cleavage site RRRR. In certain embodiments, activin A or mature activin A is human. In certain embodiments, mature activin A has the amino acid sequence between residues 311 to 426 (SEQ ID NO: 12) of activin A preproprotein (SEQ ID NO: 11).

The phrase “activin A fragment,” “activin A peptide,” or “B cell epitope of activin A,” as used herein, refers to a portion of the full-length activin A sequence that includes about 7 to about 30 (e.g., 7-15, 7-14, 7-13, 7-12, 7-11, or 7-10, or 7, 8, 9, 10, 11, 12, 13, 14, or 15) contiguous amino acid residues from the activin A sequence. The various activin A fragments described herein can be referred to by their amino acid positions in relation to the full-length sequence of preproactivin A represented by SEQ ID NO: 11. In some embodiments, an activin A fragment includes one or more amino acid changes (e.g., substitutions, deletions, or additions), for example, one or more amino acid substitutions (e.g., 1, 2, 3, 4, 5, or 6 amino acid substitutions). In some embodiments, the one or more amino acid changes, for example, the one or more (e.g., 1, 2, 3, 4, or 5) amino acid substitutions, are designed to facilitate cyclization of the activin A fragment. In some embodiments, the activin A fragment has the sequence of SEQ ID NO: 13, 14, 15, 16, 17, or 18 (see Table 2). In some embodiments, the activin A fragment has the sequence of SEQ ID NO: 110, 111, 112, 113, 114, 115, 116, 117, 118, 119, 187, or 189 (see Table 2).

### Myostatin and Activin A Peptide Analogues

The myostatin and activin A fragments of the present disclosure also include immunologically functional analogues or homologues of myostatin or activin A, or fragments thereof. Functional immunological analogues or homologues of myostatin or activin A, or fragments thereof, include variants that retain substantially the same immunogenicity as the original peptide. Immunologically functional analogues can have one or more conservative substitutions in an amino acid position; a change in overall charge; a covalent attachment to another moiety; or amino acid additions, insertions, or deletions; and/or any combination thereof.

Conservative substitutions are those in which one amino acid residue is substituted for another amino acid residue with similar chemical properties. For example, the nonpolar (hydrophobic) amino acids include alanine, leucine, isoleucine, valine, proline, phenylalanine, tryptophan, and methionine; the polar neutral amino acids include glycine, serine, threonine, cysteine, tyrosine, asparagine, and glutamine; the positively charged (basic) amino acids include arginine, lysine, and histidine; and the negatively charged (acidic) amino acids include aspartic acid and glutamic acid.

Immunologically functional analogues can include amino acid sequences that comprise conservative substitutions, additions, deletions, or insertions from one to about four amino acid residues that elicit immune responses that are cross-reactive with myostatin or myostatin fragments, or activin A or activin A fragments. The conservative substitutions, additions, and insertions can be accomplished with natural or non-natural amino acids. Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-aminohexanoic acid), hydroxyproline, mercaptopropionic acid (MPA), 3-nitro-tyrosine, pyroglutamic acid, 2,3-diaminopropionic acid, 2,4-diaminobutyric acid, ornithine, and the like. Naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In some embodiments, myostatin or activin A B cell epitopes provided herein comprise a non-naturally occurring amino acid, e.g., one or more of the following: 6-aminohexanoic acid, 2,3-diaminopropionic acid, 2,4-diaminobutyric acid, and ornithine.

In some embodiments, the immunologically functional analogues of the myostatin or activin A B cell epitope peptides comprise one or more (e.g., two) amino acid substitutions with a non-naturally occurring amino acid to allow cyclization of the peptide.

In certain embodiments, the immunologically functional analogue of a particular peptide includes the same amino acid sequence as the original peptide and further includes three lysine residues (Lys-Lys-Lys) added to the amino terminus of the myostatin or activin A peptide (or a fragment thereof). In such embodiments, the addition of three lysine residues to the original peptide sequence changes the overall charge of the original peptide but does not alter the function of the original peptide. In certain embodiments, a functional analogue of a myostatin or activin A peptide has at least 50% sequence identity to the original amino acid sequence. In other embodiments, the functional analogue has at least 80% identity to the original amino acid sequence. In yet other embodiments, the functional analogue has at least 85% identity to the original amino acid sequence. In still other embodiments, the functional analogue has at least 90% identity to the original amino acid sequence. In further embodiments, the functional analogue has at least 95% or more (e.g., at least 96%, 97%, 98%, or 99%) identity to the original amino acid sequence. The percent identity between two sequences can be determined manually by inspection of the two optimally aligned sequences or by using software programs or algorithms (e.g., BLAST, ALIGN, CLUSTAL) using standard parameters, as is known in the art. In some embodiments, the functional analogue includes 1, 2, 3, 4, 5, 6, 7, 8, 9, or 10 amino acid substitutions or deletions, as compared to the original amino acid sequence. In some embodiments, 1, 2, or 3 substitutions are included.

### Heterologous T Helper Cell Epitopes (Th Epitopes)

Peptide immunogen constructs of disclosure include a B cell epitope from myostatin or activin A (e.g., as described above) covalently linked to a heterologous T helper cell (Th) epitope directly or through an optional heterologous spacer. The heterologous Th epitopes in the myostatin or activin A peptide immunogen constructs enhance the immunogenicity of the myostatin or activin A peptides, which facilitates the production of specific high titer antibodies directed against the optimized target B cell epitopes (i.e., the myostatin peptide or the activin A peptide) through rational design.

The term “heterologous,” as used herein in reference to myostatin, refers to an amino acid sequence that is derived from an amino acid sequence that is not part of, or homologous with, the wild-type sequence of myostatin. Thus, a heterologous Th epitope is a Th epitope derived from an amino acid sequence that is not naturally found in myostatin (i.e., the Th epitope is not autologous to myostatin). Since the Th epitope is heterologous to myostatin, the natural amino acid sequence of myostatin is not extended in either the N-terminal or C-terminal directions when the heterologous Tb epitope is covalently linked to the myostatin peptide.

The term “heterologous,” as used herein in reference to activin A, refers to an amino acid sequence that is derived from an amino acid sequence that is not part of, or homologous with, the wild-type sequence of activin A. Thus, a heterologous Th epitope is a Th epitope derived from an amino acid sequence that is not naturally found in activin A (i.e., the Th epitope is not autologous to activin A). Since the Th epitope is heterologous to activin A, the natural amino acid sequence of activin A is not extended in either the N-terminal or C-terminal directions when the heterologous Th epitope is covalently linked to the activin A peptide.

The heterologous Th epitope of the present disclosure can be any Th epitope that does not have an amino acid sequence naturally found in the B cell epitope-containing peptide of the construct in which it occurs (i.e., the myostatin or activin A peptide). The Th epitope can have an amino acid sequence derived from any species (e.g., human, pig, cattle, dog, rat, mouse, guinea pigs, etc.) or from a pathogen (e.g., a measles virus or a hepatis virus (e.g., a hepatitis virus surface protein; see below)). The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e., little, if any, of the antibodies generated by the myostatin peptide or the activin A immunogen constructs will be directed towards the Th epitope(s), thus allowing a very focused immune response directed to the targeted B cell epitope of the myostatin or activin A peptide.

Th epitopes include, but are not limited to, amino acid sequences derived from foreign pathogens, as exemplified in Table 3 (SEQ ID NOs: 19-47). Further, Th epitopes include idealized artificial Th epitopes and combinatorial idealized artificial Th epitopes (e.g., SEQ ID NOs: 20 and 27-33). The heterologous Th epitope peptides presented as a combinatorial sequence (e.g., SEQ ID NOs: 28-31), contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptide assemblies can allow broad Th epitope coverage for animals having a diverse genetic background. Representative combinatorial sequences of heterologous Th epitope peptides include SEQ ID NOs: 28-31, which are shown in Table 3. Th epitope peptides of the present disclosure provide broad reactivity and immunogenicity to animals and patients from genetically diverse populations. In some embodiments, the sequence is SEQ ID NO: 30. In some embodiments, the sequence is SEQ ID NO: 32. In some embodiments, the sequence is SEQ ID NO: 33.

Peptide immunogen constructs comprising Th epitopes can optionally be produced simultaneously in a single solid-phase peptide synthesis in tandem with the B cell epitope-containing myostatin or activin A peptide.

Th epitopes also include immunological analogues of Th epitopes (see, e.g., above description of analogs). Immunological Th analogues include immune-enhancing analogs, cross-reactive analogs, and fragments of any of these Th epitopes that are sufficient to enhance or stimulate an immune response to the myostatin or activin A peptides.

Immunologically functional analogues of the Th epitope peptides are also effective and can be used in the methods of the disclosure. Immunologically functional Th analogues can include conservative substitutions, additions, deletions, and insertions of from one to about five amino acid residues (e.g., 1, 2, 3, 4, or 5) in the Th epitope which do not essentially modify the Th-stimulating function of the Th epitope. The conservative substitutions, additions, and insertions can be accomplished with natural or non-natural amino acids, which can also be the case for myostatin and activin A peptides, as described above.

Table 3 identifies another variation of a functional analogue for Th epitope peptide. In particular, SEQ ID NOs: 20 and 27 of MvF1 and MvF2 Th are functional analogues of SEQ ID NOs: 30 and 32 of MvF4 and MvF5 in that they differ in the amino acid frame by the deletion (SEQ ID NOs: 20 and 27) or the inclusion (SEQ ID NOs: 30 and 32) of two amino acids each at the N- and C-termini. The differences between these two series of analogous sequences would not affect the function of the Th epitopes contained within these sequences. Therefore, functional immunological Th analogues can, for example, include several versions of the Th epitope derived from Measles Virus Fusion protein MvF1-4 Ths (SEQ ID NOs: 20, 27, 28, 30, and 32) and from Hepatitis Surface protein HBsAg 1-3 Ths (SEQ ID NOs: 29, 31, and 33).

The Th epitope in the peptide immunogen constructs can be covalently linked at either N- or C-terminal end of the myostatin or activin A peptide. In some embodiments, the Th epitope is covalently linked to the N-terminal end of the myostatin or activin A peptide. In other embodiments, the Th epitope is covalently linked to the C-terminal end of the myostatin or activin A peptide. In certain embodiments, more than one Th epitope is covalently linked to the myostatin or activin A peptide. When more than one Th epitope is linked to the myostatin or activin A peptide, each Th epitope can have the same amino acid sequence or different amino acid sequences. In addition, when more than one Th epitope is linked to the myostatin or activin A peptide, the Th epitopes can be arranged in any order. For example, the Th epitopes can be consecutively linked to the N-terminal end of the myostatin or activin A peptide, or consecutively linked to the C-terminal end of the myostatin or activin A peptide, or a Th epitope can be covalently linked to the N-terminal end of the myostatin or activin A peptide while a separate Th epitope is covalently linked to the C-terminal end of the myostatin or activin A peptide. There is no limitation in the arrangement of the Th epitopes in relation to the myostatin or activin A peptide.

In some embodiments, the Th epitope is covalently linked to the myostatin or activin A peptide directly. In other embodiments, the Th epitope is covalently linked to the myostatin or activin A peptide through a heterologous spacer, e.g., as described in further detail below.

### Heterologous Spacer or Linker

The peptide immunogen constructs optionally include a heterologous spacer or linker that covalently links the B cell epitope from myostatin or activin A to the heterologous T helper cell (Th) epitope.

As discussed above, the term “heterologous” refers to an amino acid sequence that is derived from an amino acid sequence that is not part of, or homologous with, the wild-type sequence of myostatin, in the case of myosin peptide immunogen constructs, or activin A, in the case of activin A peptide immunogen constructs. Thus, the natural amino acid sequence of myostatin is not extended in either the N-terminal or C-terminal directions when the heterologous spacer or linker is covalently linked to the B cell epitope from myostatin because the spacer is heterologous to the myostatin sequence. Similarly, the natural amino acid sequence of activin A is not extended in either the N-terminal or C-terminal directions when the heterologous spacer or linker is covalently linked to the B cell epitope from activin A because the spacer is heterologous to the activin A sequence.

The spacer or linker is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer or linker can provide structural features to the myostatin or activin A peptide immunogen construct. Structurally, the spacer or linker provides a physical separation of the Th epitope from the B cell epitope of the myostatin or activin A fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer or linker can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences of flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 90), where Xaa is any amino acid, for example, aspartic acid. In some embodiments, the spacer comprises K-K-K, εK-K-K-K (SEQ ID NO: 91), or K-K-K-εK (SEQ ID NO: 192).

In some embodiments, the spacer is a polylysine spacer or a spacer comprising or consisting of two or more lysines. In some embodiments, the spacer comprises or consists of 2, 3 or 4 lysines (e.g., 3-4 lysines). In some embodiments, the spacer comprises an epsilon lysine linkage. In some embodiments, the spacer comprises ε-Lys (EK) or Lys-ε-Lys (K-εK). In some embodiments, the sequence of the spacer comprises ε-Lys-Lys-Lys-Lys (SEQ ID NO: 91) or Lys-Lys-Lys-ε-Lys (SEQ ID NO: 192). As is understood in the art, for a construct including ε-Lys as or within a spacer, the C-terminus of a first peptide sequence is conjugated to the epsilon amino group of the lysine to form a first amide bond, and the N-terminus of a second peptide sequence is conjugated to the carboxyl group of the lysine to form a second amide bond.

The structure of the Lys-ε-Lys (K-εK) linkage is shown below:

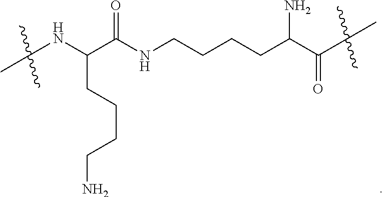 [SMILES: *NC(CCCCN)C(=O)NCCCCC(N)C(*)=O]

The spacer or linker can also provide functional features to the myostatin or activin A peptide immunogen construct. For example, the spacer or linker can be designed to change the overall charge of the myostatin or activin A peptide immunogen construct, which can affect the solubility of the peptide immunogen construct. Additionally, changing the overall charge of the myostatin or activin A peptide immunogen construct can affect the ability of the peptide immunogen construct to associate with other compounds and reagents. As discussed in further detail below, the myostatin or activin A peptide immunogen construct can be formed into a stable immunostimulatory complex with a highly charged oligonucleotide, such as CpG oligomers through electrostatic association. The overall charge of the myostatin or activin A peptide immunogen construct is important for the formation of these stable immunostimulatory complexes.

Chemical compounds that can be used as a spacer or linker include, but are not limited to (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-aminohexanoic acid), hydroxyproline, mercaptopropionic acid (MPA), 3-nitro-tyrosine, pyroglutamic acid, and the like.

The spacer or linker in a myostatin or activin A peptide immunogen construct can be covalently linked at either N- or C-terminal end of the Th epitope and the myostatin or activin A peptide. In some embodiments, the spacer is covalently linked to the C-terminal end of the Th epitope and to the N-terminal end of the myostatin or activin A peptide. In other embodiments, the spacer is covalently linked to the C-terminal end of the myostatin or activin A peptide and to the N-terminal end of the Th epitope. In certain embodiments, more than one spacer or linker can be used, for example, when more than one Th epitope is present in the peptide immunogen construct. When more than one spacer is used, each spacer can be the same as each other or different. Additionally, when more than one Th epitope is present in the peptide immunogen construct, the Th epitopes can be separated with a spacer, which can be the same as, or different from, the spacer used to separate the Th epitope from the B cell epitope. There is no limitation in the arrangement of the spacer in relation to the Th epitope or the myostatin or activin A fragment.

In certain embodiments, the heterologous spacer or linker is a naturally occurring amino acid or a non-naturally occurring amino acid. In other embodiments, the spacer contains more than one naturally occurring or non-naturally occurring amino acid (e.g., the spacer is a peptide). In specific embodiments, the spacer is Lys-, Gly-, Lys-Lys-Lys-, (α, ε-N)Lys, Lys-Lys-Lys-ε-Lys (SEQ ID NO: 192), or ε-N-Lys-Lys-Lys-Lys (SEQ ID NO: 91), or SEQ ID NO: 91 wherein the ε-Lys is on the C-terminal end of the Lys-Lys-Lys peptide.

### Multivalent or Bivalent Peptide Immunogen Constructs

In some embodiments, provided herein is a multivalent or bivalent peptide immunogen construct comprising two or more B cell epitopes, a heterologous T cell (Th) epitope, an optional heterologous linker, and an optional heterologous spacer, wherein the two or more B cell epitopes are covalently linked to the Th epitope directly or through the optional heterologous linker and/or spacer. In some embodiments, the two or more B cell epitopes are two B cell epitopes. In some embodiments, the two or more B cell epitopes are two or more (e.g., two) identical B cell epitopes, such as any B cell epitope described herein. In some embodiments, the two or more B cell epitopes are two or more (e.g., two) non-identical, different B cell epitopes, such as any two or more B cell epitopes described herein. In some embodiments, the two or more B cell epitopes are two or more (e.g., two) myostatin B cell epitopes (e.g., any myostatin B cell epitopes described herein, identical or different). In some embodiments, the two or more B cell epitopes are two or more (e.g., two) activin A B cell epitopes (e.g., any activin A B cell epitopes described herein, identical or different). In some embodiments, the two or more (e.g., two) B cell epitopes comprise at least one myostatin B cell epitope (e.g., any myostatin B cell epitope described herein) and at least one activin A B cell epitope (e.g., any activin A B cell epitope described herein).

In some embodiments, a peptide immunogen provided herein comprises two or more (e.g., 2) B cell epitopes sequentially positioned in a linear peptide.

In some embodiments, a peptide immunogen provided herein comprises two or more (e.g., 2) B cell epitopes that are positioned in a non-linear or non-sequential manner or branched off from a Th epitope. The branched design may improve or simplify peptide synthesis and/or improve or eliminate steric interactions resulting in poor interaction between the peptide and its target.

In some embodiments, the B cell epitope peptides are attached to the same side (N-terminal or C-terminal) of the Th epitope using a branched amino acid, which can be: (i) a diamino acid if the B-cell epitopes are attached to the N-terminus of a Th epitope, or (ii) a dicarboxylic acid if the B-cell epitopes are attached to the C-terminus of a Th epitope. Any diamino acid and dicarboxylic acid known in the art or described herein can be used. In some embodiments, the diamino acid is lysine. Other diamino acids include, without limitation, ornithine, 2,4-Diaminobutyric acid, and 2,3-diaminopropionic acid. In some embodiments, the dicarboxylic acid is glutamic acid. Other dicarboxylic acids include, without limitation, aspartic acid and a-amino adipic acid.

By adding more than one branched amino acid, the number of B cell epitope peptides supported by the same Th epitope can be added to a peptide immunogen provided herein. In some embodiments, the B cell epitope peptides are attached to the same side (N-terminal or C-terminal) of the Th epitope using two or more branched amino acids as described herein or known in the art.

Between the branched amino acid and the B cell epitope peptide, a heterologous linker can be used, e.g., to (i) to prevent steric interferences between the B cell epitope peptides during synthesis, and/or (ii) to avoid steric interference that would prevent either the binding of B cell receptor to B cell epitope or the binding of MHC receptor to Th epitope. In some embodiments, the linker comprises one or more amino acids (e.g., one or more Glycines). In some embodiments, the linker is an amino acid peg derivative. In some embodiments, the heterologous linker is AeeA (i.e., 8-amino-3,6-dioxaoctanoic acid or 2-[2-(2-aminoethoxy)ethoxy]acetic acid (CAS:134978-97-5)). In other embodiments, other heterologous linkers can be used, for example, and without limitation, a spacer comprising 3 to 6 pegs (e.g., 1-Amino-3,6,9,12,15,18-hexaoxahenicosan-21-oic acid (6pe), 1-amino-4,7,10,13-tetraoxapentadecanoic acid (3peg)), 6-aminohexanoic acid, or one or more epsilon-lysine. Other spacers or linkers described herein can also be used between the branched amino acid and the B cell epitope peptide.

In some embodiments, B cell epitopes can be synthesized simultaneously using a Fmoc-Lys(Fmoc)-OH on the branched amino acid. In some embodiments, B cell epitopes can be synthetized sequentially by using Lysine with orthogonal protections on the α-amino group and on ε-amino group, for example with Lysine using Fmoc-Lys(Mtt)-OH, Fmoc-Lys(DDe)-OH and Fmoc-Lys(Alloc)-OH, where different B-cell epitopes can be synthesized on the same Th epitope.

### Specific Embodiments of the Peptide Immunogen Constructs

The myostatin peptide immunogen construct can be represented by the formulae: (Th)ₘ-(A)ₙ-(myostatin fragment), or (myostatin fragment)-(A)ₙ-(Th)ₘ-, or (Th)ₘ-(A)ₙ-(myostatin fragment)-(A)ₙ-(Th)ₘ-, wherein Th is a heterologous T helper epitope; A is an optional heterologous spacer; (myostatin fragment) is a myostatin B cell epitope having about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues of myostatin (or a variant thereof, e.g., as described herein); m is an integer from 1 to about 4; and n is an integer from 0 to about 10. In some embodiments, a C-terminal X is included, which is an α-COOH or α-CONH₂ of an amino acid. In some embodiments, A is an amino acid and n indicates the number of amino acids, wherein each A can be identical to one another or one or more of the A's can be a different amino acid.

In certain embodiments, the heterologous Th epitope in the myostatin peptide immunogen construct has an amino acid sequence selected from any of SEQ ID NOs: 19-47, or combinations thereof, shown in Table 3. In specific embodiments, the Tb epitope has an amino acid sequence selected from any of SEQ ID NOs: 27-33. In some embodiments, the Th epitope has an amino acid sequence of SEQ ID NOs: 30. In some embodiments, the Th epitope has an amino acid sequence of SEQ ID NOs: 32. In certain embodiments, the myostatin peptide immunogen construct contains more than one Th epitope.

In certain embodiments, the optional heterologous spacer is selected from any of Lys-, Gly-, Lys-Lys-Lys-, (α, ε-N)Lys, ε-N-Lys-Lys-Lys-Lys (SEQ ID NO: 91), and combinations thereof. In specific embodiments, the heterologous spacer is ε-N-Lys-Lys-Lys-Lys (SEQ ID NO: 91). In some embodiments, the heterologous spacer is Lys-Lys-Lys-εLys (SEQ ID NO: 192).

In certain embodiments, the myostatin fragment has about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) amino acid residues of myostatin, corresponding to the sequence from about the D at amino acid position 267 (D267) to about the S at amino acid position 375 (S375) of full-length myostatin. In certain embodiments, the myostatin fragment has about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) amino acid residues from about the leucine at amino acid position 80 (L80) to about the leucine at amino acid position 270 (1270) of full-length myostatin preproprotein (SEQ ID NO: 1). In specific embodiments, the myostatin fragment has an amino acid sequence of a fragment of SEQ ID NO: 2, as shown in Table 1. In specific embodiments, the myostatin fragment has an amino acid sequence of a fragment of SEQ ID NO: 1, as shown in Table 1. In some embodiments, the myostatin peptide has a sequence selected from SEQ ID NOs: 3-10, as shown in Table 1. In some embodiments, the myostatin peptide has a sequence selected from SEQ ID NOs: 95-109, as shown in Table 1. In some embodiments, the myostatin peptide has a sequence selected from SEQ ID NOs: 145-164 and 166, as shown in Table 1. In some embodiments, the myostatin peptide has a sequence selected from any one of: SEQ ID NOs: 5, 97, 98, 101, 105, 146, and 147, as shown in Table 1. In certain embodiments, the myostatin peptide immunogen construct has an amino acid sequence selected from any of SEQ ID NOs: 48, 49, 50, 51, 52, 53, 54, 55, 56, 57, 58, 59, 60, 61, 62, 63, 64, 65, 66, 67, 68, 69, 70, and 71, as shown in Table 4. In certain embodiments, the myostatin peptide immunogen construct has an amino acid sequence selected from any of SEQ ID NOs: 120, 121, 122, 123, 124, 125, 126, 127, 128, 129, 130, 131, 132, 133, and 134, as shown in Table 4. In certain embodiments, the myostatin peptide immunogen construct has an amino acid sequence selected from any of SEQ ID NOs: 167, 168, 169, 170, 171, 172, 173, 174, 175, 176, 177, 178, 179, 180, 181, 182, 183, 184, 185, 186 and 188, as shown in Table 4. In certain embodiments, the myostatin peptide immunogen construct has an amino acid sequence selected from any one of: SEQ ID NOs: 54, 55, 56, 122, 123, 126, 130, 168, 169, 173, 174, 176, 177, 179, 181, 183, 185, and 186 as shown in Table 4.

The activin A peptide immunogen construct can be represented by the formulae: (Th)ₘ-(A)ₙ-(activin A fragment), or (activin A fragment)-(A)ₙ-(Th)ₘ-, or (Th)ₘ-(A)ₙ-(activin A fragment)-(A)ₙ-(Th)ₘ-, wherein Th is a heterologous T helper epitope; A is an optional heterologous spacer; (activin A fragment) is an activin A B cell epitope having about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) contiguous amino acid residues of activin A (or a variant thereof, e.g., as described herein); m is an integer from 1 to about 4; and n is an integer from 0 to about 10. In some embodiments, a C-terminal X is included, which is an α-COOH or α-CONH₂ of an amino acid. In some embodiments, A is an amino acid and n indicates the number of amino acids, wherein each A can be identical to one another or one or more of the A's can be a different amino acid.

In certain embodiments, the heterologous Th epitope in the activin A peptide immunogen construct has an amino acid sequence selected from any of SEQ ID NOs: 19-47, or combinations thereof, shown in Table 3. In specific embodiments, the Th epitope has an amino acid sequence selected from any of SEQ ID NOs: 27-33. In some embodiments, the Th epitope has an amino acid sequence of SEQ ID NOs: 30. In some embodiments, the Th epitope has an amino acid sequence of SEQ ID NOs: 32. In certain embodiments, the activin A peptide immunogen construct contains more than one Th epitope.

In certain embodiments, the optional heterologous spacer is selected from any of Lys-, Gly-, Lys-Lys-Lys-, Lys-Lys-Lys-ε-Lys (SEQ ID NO: 192), (α, ε-N)Lys, ε-N-Lys-Lys-Lys-Lys (SEQ ID NO: 91), and combinations thereof. In specific embodiments, the heterologous spacer is ε-N-Lys-Lys-Lys-Lys (SEQ ID NO: 91). In some embodiments, the heterologous spacer is Lys-Lys-Lys-ε-Lys (SEQ ID NO: 192).

In certain embodiments, the activin A fragment has about 7 to about 30 (e.g., 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30) amino acid residues of activin A. In specific embodiments, the activin A fragment has an amino acid sequence represented by SEQ ID NO: 12, as shown in Table 1. In some embodiments, the activin A peptide has a sequence selected from SEQ ID NOs: 13-18, as shown in Table 2. In some embodiments, the activin A peptide has a sequence selected from SEQ ID NOs: 110-119, 187, and 189, as shown in Table 2. In some embodiments, the activin A peptide has a sequence selected from any one of: SEQ ID NOs: 111, 116, 118 and 119, as shown in Table 2. In certain embodiments, the activin A peptide immunogen construct has an amino acid sequence selected from any of SEQ ID NOs: 72-89, as shown in Table 5. In certain embodiments, the activin A peptide immunogen construct has an amino acid sequence selected from any of SEQ ID NOs: 135, 136, 137, 138, 139, 140, 141, 142, 143, 144, 190, and 191 as shown in Table 5. In certain embodiments, the activin A peptide immunogen construct has an amino acid sequence selected from any one of: SEQ ID NOs: 136, 141, 143, 144, and 190 as shown in Table 5.

### Compositions

Peptide immunogen constructs and related molecules described herein can be comprised with compositions, including pharmaceutical compositions, which are capable of eliciting an immune response and the production of antibodies against the peptide immunogen constructs in a subject (e.g., a human patient). The disclosed compositions can include one or a mixture of more than one of the peptide immunogen constructs. In some embodiments, the compositions include one or more myostatin peptide immunogen construct described herein. In some embodiments, the compositions include one or more activin A peptide immunogen construct described herein. In some embodiments, the compositions include one or more myostatin peptide immunogen construct described herein and one or more activin A peptide immunogen construct described herein. In some embodiments, the compositions include one myostatin peptide immunogen construct described herein and one activin A peptide immunogen construct described herein. As one example, the compositions can include construct peptide (SEQ ID NO: 55) as described herein. As another example, the composition can include a myostatin peptide immunogen construct selected from Table 4 (optionally wherein the myostatin peptide portion of the construct is 7-30, 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27, 28, 29, or 30 amino acids in length), together with an activin A peptide immunogen construct selected from Table 5 (optionally wherein the activin A peptide portion of the construct is 7-30, 7-29, 7-28, 7-27, 7-26, 7-25, 7-24, 7-23, 7-22, 7-21, 7-20, 7-19, 7-18, 7-19, 7-18, 7-17, 7-16, 7-15, 7-14, 7-13, 7-12, 7-11, or 7-10, or 7, 8, 9, 10, 11, 12, 13, 14, 15, 16, 17, 18, 19, 20, 21, 22, 23, 24, 25, 26, 27 28, 29, or 30 amino acids in length). As another example, the composition can include, a myostatin peptide immunogen construct of SEQ ID NO: 54, 55, 56, 122, 123, 126, 130, 168, 169, 173, 174, 176, 177, 179, 181, 183, 185, or 186 (or another myostatin peptide immunogen construct described herein), and an activin A peptide immunogen construct of SEQ ID NO: 136, 141, 143, 144, or 190 (or another activin A peptide immunogen construct described herein).

Furthermore, in some embodiments, the compositions can include the peptide immunogen construct(s) together with one or more additional component, e.g., carriers, adjuvants, buffers, and/or other suitable reagents. In some embodiments, the compositions include the peptide immunogen constructs in the form of a stabilized immunostimulatory complex with a CpG oligomer that is optionally supplemented with an adjuvant.

Compositions containing one or more disclosed peptide immunogen construct can be in liquid or solid form. Liquid compositions can include water, buffers, solvents, salts, and/or any other acceptable reagent that does not alter the structural or functional properties of the peptide immunogen constructs.

### Pharmaceutical Compositions

The present disclosure includes pharmaceutical compositions containing the disclosed peptide immunogen construct(s).

Pharmaceutical compositions can contain carriers and/or other additives in a pharmaceutically acceptable delivery system. Accordingly, pharmaceutical compositions can contain a pharmaceutically effective amount of one or more peptide immunogen construct together with pharmaceutically acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the peptide immunogen construct(s) without having any specific antigenic effect itself. Adjuvants used in the pharmaceutical composition can include oils, aluminum salts, virosomes, aluminum phosphate (e.g., ADJU-PHOS®), aluminum hydroxide (e.g., ALHYDROGEL®), liposyn, saponin, squalene, L121, Emulsigen®, monophosphoryl lipid A (MPL), QS21, ISA 35, ISA 206, ISA50V, ISA51, ISA 720, as well as the other adjuvants and emulsifiers.

In some embodiments, the pharmaceutical composition contains Montanide™ ISA 51 (an oil adjuvant composition comprised of vegetable oil and mannide oleate for production of water-in-oil emulsions). Tween® 80 (also known as polysorbate 80 or polyoxyethylene (20) sorbitan monooleate), a CpG oligonucleotide, and/or any combination thereof. In other embodiments, the pharmaceutical composition is a water-in-oil-in-water (i.e., w/o/w) emulsion with Emulsigen or Emulsigen D as the adjuvant.

Pharmaceutical compositions can be formulated for immediate release or for sustained release. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the peptide immunogen constructs can also be prepared prior to injection. The pharmaceutical compositions can be administered by any suitable mode of application, for example, intramuscularly, subcutaneously, intradermally, intravenously, intraperitoneally, intranasally, orally, etc. and by use of any suitable formulation or delivery device.

Pharmaceutical compositions can also be formulated in a suitable dosage unit form. In some embodiments, the pharmaceutical composition contains from about 0.5 μg to about 1 mg of the peptide immunogen construct(s) per kg body weight. In some embodiments, the pharmaceutical composition contains 10-1000 μg, e.g., 20-500 μg, 50-400 μg, 50-500 μg, 100-400 μg, or 100-300 kg of an immunotherapy as described herein (e.g., a peptide immunogen construct(s)). In some embodiments, the indicated dose is the total dose of one or more peptide immunogen constructs administered. In some embodiments where more than one peptide immunogen construct are administered, each of the peptide immunogen constructs are administered in about equal amount by weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human, but non-human mammals, including cattle, pigs, goats, sheep, horses, dogs, cats, and transgenic mammals can also be treated. When delivered in multiple doses, the pharmaceutical compositions may be conveniently divided into an appropriate amount per dosage unit form as determined to be appropriate by one skilled in the art. The administered dosage will depend on the age, weight, and general health of the subject as is well known in the therapeutic arts.

In some embodiments, the pharmaceutical composition contains more than one peptide immunogen construct and/or antibody. A pharmaceutical composition containing a mixture of more than one peptide immunogen construct (and/or antibody) can allow for synergistic enhancement of the immunoefficacy of the constructs. Pharmaceutical compositions containing more than one peptide immunogen construct can be more effective in a larger genetic population due to a broad MHC class II coverage thus provide an improved immune response to the peptide immunogen constructs.

In some embodiments, the pharmaceutical composition contains one or more myostatin peptide immunogen constructs described herein and/or one or more activin A peptide immunogen constructs described herein.

In some embodiments, the pharmaceutical composition contains a myostatin peptide immunogen construct selected from SEQ ID NOs: 48-71, 120-134, 167-186, and 188, as well as homologues, analogues, fragments, and/or combinations thereof. In specific embodiments, pharmaceutical compositions contain a myostatin peptide immunogen construct selected from SEQ ID NOs: 48-71, 120-134, 167-186, and 188, and any combination thereof. In some embodiments, pharmaceutical compositions contain a myostatin peptide immunogen construct selected from SEQ ID NO: 54, 55, 56, 122, 123, 126, 130, 168, 169, 173, 174, 175, 176, 177, 178, 179, 181, 183, 185, and 186, and any combination thereof. In some embodiments, pharmaceutical compositions contain a myostatin peptide immunogen construct selected from SEQ ID NO: 54, 55, 56, 122, 123, 126 and 130.

In some embodiments, the pharmaceutical composition contains an activin A peptide immunogen construct selected from SEQ ID NOs: 72-89, 135-144, and 190-191, as well as homologues, analogues, fragments, and/or combinations thereof. In specific embodiments, pharmaceutical compositions contain an activin A peptide immunogen construct selected from SEQ ID NOs: 72-89, 135-144, and 190-191, and any combination thereof. In some embodiments, pharmaceutical compositions contain an activin A peptide immunogen construct selected from SEQ ID NO: 75, 76, 78, 79, 85, 136, 138, 139, 140, 141, 142, 143, 144, and 190, and any combination thereof.

In some embodiments, the pharmaceutical composition contains a myostatin peptide immunogen construct and an activin A peptide immunogen construct.

In some embodiments, the pharmaceutical composition contains a myostatin peptide immunogen construct selected from SEQ ID NOs: 48-71, 120-134, 167-186 and 188, as well as homologues, analogues, fragments, and/or combinations thereof, and an activin A peptide immunogen construct selected from SEQ ID NOs:72-89 and 135-144, as well as homologues, analogues, fragments, and/or combinations thereof. In specific embodiments, pharmaceutical compositions contain a myostatin peptide immunogen construct selected from SEQ ID NOs: 48-71, 120-134, 167-186 and 188, and any combination thereof, and an activin A peptide immunogen construct selected from SEQ ID NOs: 72-89, 135-144, and 190-191. In some embodiments, the pharmaceutical composition contains a myostatin peptide immunogen construct selected from SEQ ID NO: 54, 55, 56, 122, 123, 126, 130, 168, 169, 173, 174, 175, 176, 177, 178, 179, 181, 183, 185, and 186, as well as homologues, analogues, fragments, and/or combinations thereof, and an activin A peptide immunogen construct selected from SEQ ID NO: 75, 76, 78, 79, 85, 136, 138, 139, 140, 141, 142, 143, 144, and 190, as well as homologues, analogues, fragments, and/or combinations thereof.

Pharmaceutical compositions containing a myostatin and/or activin A peptide immunogen construct can be used to elicit an immune response and to produce antibodies in a subject upon administration. In some embodiments, a pharmaceutical composition as described herein is administered to a subject 1, 2, 3, 4, 5, or more times as determined to be appropriate by those of skill in the art. The compositions can be administered, for example, in an initial dose, followed by 1 or more (e.g., 2, 3, 4, 5, or more) booster doses. In some embodiments, an initial dose is administered in week 1 and then is followed by a dose at week 5 and an additional dose at week 13. In some embodiments, the amount of each dose is the same (e.g., 100 μg or 300 μg; also see above). In some embodiments, the amount of each dose varies, as can be determined to be appropriate by those of skill in the art. For example, the initial dose may be lower, e.g., 40 μg, followed by higher doses of 100 μg, 300 μg, 500 μg, or 1000 μg in subsequent administrations (e.g., at weeks 5 and 13). In some embodiments, a pharmaceutical composition as described herein is administered to a subject for a period of time, e.g., for at least or more than 3 months, 6 months, 9 months, 1 year, 1.5 years, 2 years, 3 years, 5 years, or 10 years. In some embodiments, a pharmaceutical composition as described herein is administered to a subject in an initial dose or doses (e.g., in the first 1-3 months) followed by booster doses (e.g., every 1-6 months) administered for a period of time, e.g., for at least or more than 6 months, 9 months, 1 year, 1.5 years, 2 years, 3 years, 5 years, or 10 years.

### Immunostimulatory Complexes

The compositions of the present disclosure can also contain one or more peptide immunogen construct in the form of an immunostimulatory complex with a CpG oligonucleotide. Such immunostimulatory complexes are specifically adapted to act as an adjuvant and as a peptide immunogen stabilizer. The immunostimulatory complexes are in particulate form, which can efficiently present the peptide immunogen(s) to the cells of the immune system to produce an immune response. The immunostimulatory complexes may be formulated as a suspension for parenteral administration. The immunostimulatory complexes may also be formulated in the form of water in oil emulsions, as a suspension in combination with a mineral salt or with an in-situ gelling polymer for the efficient delivery of the peptide immunogen(s) to the cells of the immune system of a subject following parenteral administration.

The stabilized immunostimulatory complex can be formed by complexing one or more peptide immunogen construct with an anionic molecule, oligonucleotide, polynucleotide, or combinations thereof via electrostatic association. The stabilized immunostimulatory complexes may be incorporated into a pharmaceutical composition as an immunogen delivery system.

In certain embodiments, the one or more peptide immunogen construct is designed to contain a cationic portion that is positively charged at a pH in the range of 5.0 to 8.0. The net charge on the cationic portion of the peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the peptide immunogen construct and expressed as the net average charge. A suitable peptide immunogen has a cationic portion with a net average positive charge of +1. In some embodiments, the peptide immunogen has a net positive charge in a range that is larger than +2. In some embodiments, the cationic portion of the peptide immunogen construct is the heterologous spacer. In certain embodiments, the cationic portion of the peptide immunogen construct has a charge of +4 when the spacer sequence is (α, ε-N)Lys, ε-N-Lys-Lys-Lys-Lys (SEQ ID NO: 91).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. In some embodiments, the CpG immunostimulatory single-stranded DNA molecules contain 18-48 nucleotide bases, with the number of repeats of CpG motif in the range of 3 to 8.

In some embodiments, the anionic oligonucleotide is represented by the formula: 5′X¹CGX²3′ wherein C and G are unmethylated; and X¹ is selected from A (adenine), G (guanine) and T (thymine); and X² is C (cytosine) or T (thymine). In other embodiments, the anionic oligonucleotide is represented by the formula: 5′ (X³)2CG(X⁴) 3′ wherein C and G are unmethylated; and X³ is selected from A, T, or G; and X⁴ is C or T.

In some embodiments, the CpG oligonucleotide is as described in WO 03/068169, the contents of which are incorporated by reference. In some embodiments, the CpG comprises or consists of a sequence of CpG1 (tcgtcgtttt gtcgttttgt cgttttgtcg tt; SEQ ID NO: 92), CpG2 (tcgtcgtttt gtcgttttgt cgtt; SEQ ID NO: 93), or CpG3 (tcgtcgtttt gtcgttttgt cgtt; SEQ ID NO: 94). In some embodiments, the CpG oligonucleotide (e.g., CpG1, CpG2, or CpG3) is a phosphorothioate oligonucleotide.

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions, and polymeric gels.

### Methods of Making Peptide Immunogen Constructs

The peptide immunogen constructs of the disclosure can be made using chemical synthesis methods that are well known in the art (see, e.g., Fields et al., Chapter 3 in Synthetic Peptides: A User's Guide, ed. Grant, W. H. Freeman & Co., New York, NY, 1992, p. 77). For example, the peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of peptide immunogen constructs comprising combinatorial library peptides for Th epitopes can be accomplished by providing a mixture of alternative amino acids for coupling at a given variable position. After complete assembly of a desired peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to those of skill in the art. The quality of peptides produced by this chemical process can be controlled and defined and, as a result, reproducibility of peptide immunogen constructs, immunogenicity, and yield can be assured.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically derived drugs. Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The peptide immunogen constructs can also be made using recombinant DNA technology including using nucleic acid molecules, vectors, and/or host cells. As such, nucleic acid molecules encoding the peptide immunogen constructs and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the peptide immunogen constructs and immunologically functional analogues of the myostatin or activin A fragment derived peptide immunogen constructs. For example, methods can include a step of incubating a host cell containing an expression vector containing a nucleic acid molecule encoding a peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

### Methods for Manufacturing of Immunostimulatory Complexes

As noted above, the compositions and methods of the disclosure can further include or employ immunostimulatory complexes comprising peptide immunogen constructs and CpG oligodeoxynucleotide (ODN) molecules. Stabilized immunostimulatory complexes (ISC) are derived from a cationic portion of the peptide immunogen construct and a polyanionic CpG ODN molecule. The self-assembling system is driven by electrostatic neutralization of charge. Stoichiometry of the molar charge ratio of cationic portion of the peptide immunogen construct to anionic oligomer determines extent of association. The non-covalent electrostatic association of peptide immunogen construct and CpG ODN is a completely reproducible process. The peptide/CpG ODN immunostimulatory complex aggregates, which facilitate presentation to the “professional” antigen-presenting cells (APC) of the immune system thus further enhancing of the immunogenicity of the complexes. These complexes are easily characterized for quality control during manufacturing. The peptide/CpG ISC are well tolerated in vivo. This particulate system comprising CpG ODN and fragment derived peptide immunogen constructs was designed to take advantage of the generalized B cell mitogenicity associated with CpG ODN use and yet promote balanced Th-1/Th-2 type responses.

The CpG ODN in the disclosed pharmaceutical compositions can be 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen-presenting cells (APC). Consequently, such formulations offer potential advantages by promoting the stimulation of immune responses by alternative mechanisms.

### Antibodies

The disclosure also provides antibodies that specifically recognize and bind to myostatin and/or activin A, for example, to a B cell epitope portion of a peptide immunogen construct described herein. In some embodiments, the provided antibodies are elicited in response to vaccination with one or more peptide immunogen constructs described herein. In some embodiments, the antibodies elicited by administration of one or more peptide immunogen constructs described herein to a subject are harvested or isolated from the subject, and optionally purified.

Antibodies for use in therapy can be generated using standard methods in the art and include, e.g., monoclonal antibodies, polyclonal antibodies, multispecific antibodies (e.g., bispecific and trispecific antibodies), and antibody fragments, provided that the desired antigen-binding activity and specificity is maintained. Antibody fragments include, for example, Fv, single-chain Fv (scFv), Fab, Fab′, di-scFv, sdAb (single domain antibody), and (Fab′)₂ (including a chemically linked F(ab′)₂). Antibodies also include, e.g., chimeric antibodies, humanized antibodies, and antibodies of various species such as mouse, human, cynomolgus monkey, etc. Furthermore, antibody variants having the sequences from other organisms are also included. Antibody fragments also include either orientation of single chain scFvs, tandem di-scFv, diabodies, tandem tri-sdcFv, minibodies, etc. Antibody fragments further include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

### Methods of Treatment

The present disclosure provides methods for treating, delaying, lessening, preventing, and/or improving one or more symptoms of a muscle wasting disease or condition, or insulin resistance, or glucose metabolism, or metabolic disease, using the disclosed immunotherapies (e.g., peptide immunogen constructs (and related compositions and complexes), nucleic acid molecules, vectors, and/or antibodies directed against the B cell epitope of the peptide immunogen constructs). In some embodiments, the methods include administering to a subject a composition containing one or more of the disclosed peptide immunogen constructs. In some embodiments, the methods include administering to a subject a composition containing one or more disclosed antibodies. In certain embodiments, the compositions utilized in the methods contain one or more disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to subjects with muscle wasting diseases or conditions, or insulin resistance. The disclosed methods also include dosing regimens, dosage forms, and routes for administering the peptide immunogen constructs to a subject at risk for, or with, a muscle wasting disease or condition, or insulin resistance. As explained above, use of one or more myostatin peptide immunogen construct in combination with one or more activin A peptide immunogen construct is included in the disclosure.

The present disclosure also provides methods for maintaining or improving muscle physiology using the disclosed immunotherapies (e.g., peptide immunogen constructs (and related compositions and complexes), nucleic acid molecules, vectors, and/or antibodies directed against the B cell epitope of the peptide immunogen constructs). In some embodiments, the methods include administering to a subject a composition containing one or more disclosed peptide immunogen constructs. In some embodiments, provided herein are methods of increasing muscle mass or muscle growth using the peptide immunogen constructs described herein. In some embodiments, provided herein are methods of increasing muscle strength using the peptide immunogen constructs described herein. In some embodiments, muscle strength or muscle performance is measured by any method known in the art or described herein. In some embodiments, muscle strength is measured by measuring grip strength by any method described herein or known in the art. In some embodiments, provided herein are methods of increasing muscle tone using the peptide immunogen constructs described herein. In some embodiments, provided herein are methods of increasing muscle endurance using the peptide immunogen constructs described herein. In some embodiments, provided herein are methods of improving muscle function or performance using the peptide immunogen constructs described herein.

The present disclosure also provides methods for treating, delaying, lessening, preventing, and/or improving one or more symptoms of muscular or musculoskeletal injury using the disclosed immunotherapies (e.g., peptide immunogen constructs (and related compositions and complexes), nucleic acid molecules, vectors, and/or antibodies directed against the B cell epitope of the peptide immunogen constructs). In some embodiments, the methods include administering to a subject a composition containing one or more disclosed peptide immunogen constructs. In some embodiments, provided herein are methods of preventing or treating muscular injury using the peptide immunogen constructs described herein. In some embodiments, provided herein are methods of preventing or treating musculoskeletal injury using the peptide immunogen constructs described herein. In some embodiments, the muscular or musculoskeletal injury is a muscle tear or muscle strain.

The present disclosure also provides methods for treating, delaying, lessening, preventing, and/or improving one or more symptoms of muscle loss or insufficient muscle growth using the disclosed immunotherapies (e.g., peptide immunogen constructs (and related compositions and complexes), nucleic acid molecules, vectors, and/or antibodies directed against the B cell epitope of the peptide immunogen constructs). In some embodiments, the methods include administering to a subject a composition containing one or more disclosed peptide immunogen constructs. In some embodiments, provided herein are methods of preventing or treating muscle loss using the peptide immunogen constructs described herein. In some embodiments, provided herein are methods of preventing or treating insufficient muscle growth using the peptide immunogen constructs described herein. In some embodiments, provided herein are methods of preventing or treating muscle atrophy using the peptide immunogen constructs described herein. In some embodiments, provided herein are methods of preventing or treating cachexia or muscle loss associated with a disease or condition (e.g., cancer) using the peptide immunogen constructs described herein. In some embodiments, provided herein are methods of preventing or treating age- or disease-related muscle loss in a subject using the peptide immunogen constructs described herein (e.g., wherein the disease is cancer). In some embodiments, the subject is 55 years of age or older. In some embodiments, the subject is 60 years of age or older. In some embodiments, the subject is 65 years of age or older. In some embodiments, the subject is 70 years of age or older. In some embodiments, the subject is 75 years of age or older. In some embodiments, the subject is 80 years of age or older. In some embodiments, the subject is 85 years of age or older. In some embodiments, the subject exhibits a deficit or a severe deficiency in the G-H and/or IGF-1 due to aging. In some embodiments, the subject exhibits a deficit in GH which does not allow GH-associated muscle growth or leads to a decreased or insubstantial GH-associated muscle growth. In some of these embodiments, the subject is a human female. In some embodiments, provided herein are methods of preventing or treating sarcopenia in a subject using the peptide immunogen constructs described herein (e.g., wherein the disease is cancer). In some embodiments, administering of peptide immunogen constructs described herein prevents loss or increases lean body mass in a subject. In some embodiments, administering of peptide immunogen constructs described herein increases muscle strength and/or performance.

The present disclosure also provides methods for treating, delaying, lessening, preventing, and/or improving metabolic disease or one or more symptoms of metabolic disease using the disclosed immunotherapies (e.g., peptide immunogen constructs (and related compositions and complexes), nucleic acid molecules, vectors, and/or antibodies directed against the B cell epitope of the peptide immunogen constructs). In some embodiments, the methods include administering to a subject a composition containing one or more disclosed peptide immunogen constructs. In some embodiments, provided herein are methods of preventing or treating metabolic disease using the peptide immunogen constructs described herein.

In some embodiments, provided herein are methods of maintaining or improving glucose metabolism using the disclosed immunotherapies (e.g., peptide immunogen constructs (and related compositions and complexes), nucleic acid molecules, vectors, and/or antibodies directed against the B cell epitope of the peptide immunogen constructs). In some embodiments, the methods include administering to a subject a composition containing one or more disclosed peptide immunogen constructs. In some embodiments, provided herein are methods of maintaining or improving glucose metabolism using the peptide immunogen constructs described herein.

In some embodiments, provided herein are methods of treating or preventing insulin resistance using the disclosed immunotherapies (e.g., peptide immunogen constructs (and related compositions and complexes), nucleic acid molecules, vectors, and/or antibodies directed against the B cell epitope of the peptide immunogen constructs). In some embodiments, the methods include administering to a subject a composition containing one or more disclosed peptide immunogen constructs. In some embodiments, provided herein are methods of treating or preventing insulin resistance using the peptide immunogen constructs described herein.

As used herein, the term “preventing” a disease or condition in a subject refers to administering a medicament (e.g., the peptide immunogen construct described herein) to the subject prior to the onset of the disease or condition, when administration of the medicament to a statistical sample prior to the onset of the disease or condition reduces the occurrence of the disease or condition in the treated sample relative to an untreated control sample, or delays the onset or reduces the occurrence or severity of one or more symptoms of the disease or condition relative to the untreated control sample.

As used herein, the term “treating” a disease or condition in a subject refers to administering a medicament (e.g., the peptide immunogen construct described herein) to the subject having or suspected of having a disease or condition (i.e., after the onset of the disease or condition), such that at least one symptom of the disease or condition is decreased or prevented from worsening.

As used herein, a “subject” is a human or a non-human animal. This term includes mammals, such as humans, primates, livestock animals, companion animals and animal models of disease.

As used herein, “administering” or “administration of” to a subject can be carried out using one of a variety of methods known to those skilled in the art. In some embodiments, the administration is parenteral. In some embodiments, the administration is intravenous, subcutaneous, intramuscular, intradermal, or intraperitoneal. For example, peptide immunogens described herein can be administered intramuscularly. In some embodiments, the pharmaceutical compositions are formulated for any of the administration methods described herein, e.g., for intramuscular (IM) administration. Administering can also be performed, for example, once, a plurality of times, and/or over one or more extended periods.

As used herein, the term an “effective amount” or a “therapeutically effective amount” is an amount of a peptide immunogen that, when administered to a subject will have or expected to have a therapeutic effect. The therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated.

In some embodiments, the subject being treated in accordance with the methods described herein is a human. Subjects who can be treated according to the methods of the disclosure include patients, such as human patients, who have or are at risk of developing a muscle wasting disease or condition such as one or more disease or condition described herein. Symptoms that can be improved according to the methods of the disclosure include, for example, one or more symptom of muscle wasting such as one or more of those listed below. The subjects can also include patients, such as human patients, who have or are at risk of developing insulin resistance. The human patients who can be treated according to the methods of the disclosure include pediatric, teen, adult, and elderly patients. Non-human animals, e.g., those described elsewhere herein, can also be treated according to the methods of the disclosure. In some embodiments, the subject is a mammal. In some embodiments, the subject is a farm animal (e.g., a cow, a pig, a goat, or a sheep). In some embodiments, the subject is a domestic animal (e.g., a dog or a cat). In some embodiments, the subject is a human.

In some embodiments, the subject is at least or over 55 years old, 60 years old, 65 years old, 70 years old, 75 years old, 80 years old, or 85 years old. In some embodiments, the subject is at least or over 55 years old, 60 years old, 65 years old, 70 years old, 75 years old, 80 years old, or 85 years old, in particular where the treatment in accordance with the methods described herein is for improving muscle strength and/or muscle performance (optionally without increasing lean mass or reducing fat mass). In some embodiments, the subject is suffering from sarcopenia or muscle wasting. In some embodiments, the subject is a human female, e.g., a female over 55, 60, 65, 70 or 80 years of age, optionally suffering from sarcopenia or muscle wasting. In some embodiments, the subject is a human male, e.g., a male over 55, 60, 65, 70 or 80 years of age, optionally suffering from sarcopenia or muscle wasting.

In some embodiments, the subject is of any age, e.g., any age over 15, 16, 17 or 18. In some embodiments, the subject is of any age, e.g., any age over 18 and does not have a growth hormone deficiency or does not have a substantial reduction in growth hormone levels. In some embodiments, the subject is younger than 55 years old, 60 years old, 65 years old, 70 years old, 75 years old, 80 years old, or 85 years old. In some embodiments, the subject is younger than 60 years of age. In some embodiments, the subject is younger than 70 years of age. In some embodiments, the subject is younger than 80 years of age. In some embodiments, the subject is older than 55 years old, 60 years old, or older than 65 years old. In some embodiments, the subject is of any age, e.g., any age over 18 but younger than 60 or 65, in particular where the treatment in accordance with the methods described herein is for increasing lean mass and/or reducing fat mass (and optionally further for improving muscle strength and/or muscle performance). In some embodiments, the subject is suffering from sarcopenia or muscle wasting. In some embodiments, the subject is suffering from obesity and/or a metabolic disease, disorder or syndrome. In some embodiments, the subject is a human female, optionally suffering from obesity and/or a metabolic disease, disorder or syndrome. In some embodiments, the subject is a human male, optionally suffering from obesity and/or metabolic disease, disorder or syndrome.

Muscle wasting can be caused by diseases and conditions of the muscle, as well as diseases and conditions of the nerves that control muscles. In addition, muscle wasting can be caused by injury (e.g., injury to a muscle or nerve), lack of use of a muscle, exposure to a toxin, and/or a systemic disease. Furthermore, the cause of muscle wasting may be genetic, acquired, direct, or indirect. The methods and compositions of the disclosure can be used in the treatment or prevention of any of these types of diseases or conditions (e.g., as described herein), as well as to prevent, treat, inhibit, or ameliorate one or more symptom thereof (e.g., as described herein). Moreover, it should be understood that, in some embodiments, treatment according to the present disclosure may be effective against one or more symptoms of a muscle wasting disease, while not addressing the underlying cause. It should also be understood that, in some embodiments, treatment according to the present disclosure may be carried out in combination with other therapies directed at treating the underlying cause of the muscle wasting and/or one or more symptoms thereof.

In more detail, muscle wasting diseases or conditions that can be treated according to the methods of the disclosure include those that are caused, for example, by inherited or genetic causes, inflammation, infection, injury, systemic diseases, toxins, and other environmental exposures. Specific, non-limiting examples of muscle wasting diseases or conditions that can be treated according to the methods of the disclosure are provided, as follows.

Inherited or genetic myopathies include, for example, muscular dystrophy (e.g., Duchenne Muscular Dystrophy (DMD) and Becker Muscular Dystrophy (BMD), myotonic dystrophy (e.g., DM1 and DM2), facioscapulohumeral muscular dystrophy, oculopharyngeal muscular dystrophy, and limb girdle muscular dystrophy), congenital myopathy (e.g., nemaline myopathy and central core myopathy), metabolic myopathy (e.g., acid maltase or acid alpha-1, 4-glucosidase deficiency (Pompe's disease), glycogen storage disorders 3-11, carnitine deficiency, fatty acid oxidation defects, and carnitine palmitoyl transferase deficiency), and mitochondrial myopathy. Autoimmune or inflammatory myopathies (i.e., myositis) include, e.g., dermatomyositis, polymyositis, inclusion body myositis, and juvenile myositis, as well as other autoimmune disorders (e.g., myasthenia gravis). Infections that may lead to muscle wasting include, for example, infections by viruses (e.g., HIV, influenza virus, and Epstein-Barr virus), bacteria (e.g., pyomyositis, S. aureus, and streptococci), spirochetes (e.g., Lyme disease), and parasites (e.g., trichinosis). Toxins that can cause myopathies include, for example, certain medications (e.g., cholesterol-lowering medications (e.g., statins), propofol, amiodaron, colchicine, chloroquine, antivirals, protease inhibitors, omeprazole, and tryptophan), and toxins such as alcohol and toluene. Systemic diseases can also lead to muscle wasting, e.g., endocrine disorders (e.g., thyroid, parathyroid, pituitary, or adrenal disorders), systemic inflammatory disease (e.g., systemic lupus erythematosus, rheumatoid arthritis, scleroderma, Sjogren's syndrome, mixed connective disease, and sarcoidosis), electrolyte imbalance, critical illness myopathy, and amyloid myopathy.

Neurological diseases and conditions that may be characterized by muscle wasting include, for example, amyotrophic lateral sclerosis (ALS), multiple sclerosis (MS), spinal muscular atrophy (SMA), Guillain-Barre syndrome, carpel tunnel syndrome, Charcot-Marie-Tooth disease, Parkinson's disease, Lewy body disease, polio, spinal cord injury, peripheral nerve injury, stroke, and nerve damage caused by diabetes, toxins, or alcohol.

In addition to the above, being present in an environment with decreased or no gravity, e.g., during a space flight or being at a space station or the like, can lead to muscle wasting.

Other diseases or conditions associated with muscle wasting include prolonged inactivity (e.g., in those who are bedridden, have seated jobs or a sedentary lifestyle, or cannot move limbs due to stroke or other brain disease), burns, long-term corticosteroid therapy, malnutrition, anorexia, cachexia, osteoarthritis, age, cancer, chronic kidney disease, heart failure, and chronic obstructive pulmonary disease.

Symptoms that can be prevented, treated, inhibited, or ameliorated according to the invention include, for example, muscle loss, movement issues, balance problems, trouble walking, trouble using arms or legs, contractures, muscle rigidity, muscle twitching, fasciculations, muscle cramps, numbness, tingling, painful sensations, breathing problems, curved spine, heart problems, swallowing problems (which may lead to, e.g., nutritional problems and aspiration pneumonia), reduced muscle mass (e.g., in general or in one limb), weakness (e.g., in general or in one limb), pain, discomfort, one arm or leg being smaller than the other, weakness in one arm or leg, numbness or tingling in an arm or leg, trouble walking, balance problems, facia weakness, gradual memory loss, reduced muscle strength, impaired ability to perform physical activities, decrease in muscle size, cramps, stiffness, spasms, fatigue, motor delay, respiratory impairment, and bulbar muscle dysfunction.

In some embodiments, symptoms that can be prevented, treated, inhibited, or ameliorated according to the invention include immobilization, prolonged bed rest, and injury associated with muscle wasting. In some embodiments, the methods and compositions provided herein delay, decrease or stop progressive loss of muscle performance or muscle strength due to aging. In some embodiments, the methods and compositions provided herein improve skeletal muscle performance, e.g., in subjects suffering from muscle wasting, sarcopenia, or age-related reduction in skeletal muscle performance. In some embodiments, the methods and compositions provided herein improve the contractility of the skeletal muscle, e.g., in subjects suffering from muscle wasting, sarcopenia, or age-related decrease in skeletal muscle contractility. In some embodiments, the subject treated in accordance with the methods described herein exhibits naturally occurring growth hormone secretion decline.

Furthermore, as noted above, the methods and compositions of the disclosure can be used to prevent or treat insulin resistance in a subject, such as a human patient described herein. In some embodiments, the subject has or is at risk of developing type II diabetes or pre-diabetes. In some embodiments, the subject has or is at risk of developing metabolic syndrome. In some embodiments, the subject has or is at risk of developing hypertension, hyperglycemia, hypercholesterolemia, high triglycerides, overweight, and/or obesity. In some embodiments, treatment according to the present disclosure improves one or more symptom in such subjects, e.g., decreases insulin resistance, reduces blood glucose levels, reduces A1C levels, and/or decreases or normalizes blood pressure, cholesterol levels, and/or triglycerides.

The above-described methods comprise administering a pharmaceutical composition comprising a pharmacologically effective amount of one or more immunotherapy targeting myostatin and/or a pharmacologically effective amount of an immunotherapy targeting activin A (e.g., one or more peptide immunogen construct and/or an antibody, e.g., as described herein) to a subject in need thereof. The amounts and regimens used in the methods can be consistent with the information provided above in the section concerning compositions or as determined to be appropriate by those of skill in the art.

In some embodiments, one or more peptide immunogen construct described herein is administered to a subject 1, 2, 3, 4, 5, or more times as determined to be appropriate by those of skill in the art. The compositions can be administered, for example, in an initial dose, followed by 1 or more (e.g., 2, 3, 4, 5, 6, 7, 8, 9, 10, or more) booster doses. In some embodiments, administration of booster doses can continue long-term, e.g., for at least or more than 6 months, 9 months, 1 year, 1.5 years, 2 years, 3 years, 5 years, or 10 years. In some embodiments, an initial dose is administered in week 1 and then is followed by a dose, e.g., at week 2-6, e.g., week 5, and an additional dose later, e.g., at week 10-20, e.g., week 13. In some embodiments, the amount of each dose is the same (e.g., 100 μg or 300 μg; also see above). In some embodiments, the amount of each dose varies, as can be determined to be appropriate by those of skill in the art. For example, the initial dose may be lower, e.g., 40 μg, followed by higher doses of 100 μg, 300 μg, 500 μg, or 1000 μg in subsequent administrations. In other embodiments, the initial dose may be higher (e.g., 300 μg), followed by lower doses (e.g., 100 μg or less). In some embodiments, one or more peptide immunogen construct described herein is administered to a subject for a period of time, e.g., for at least or more than 6 months, 9 months, 1 year, 1.5 years, 2 years, 3 years, 5 years, or 10 years. In some embodiments, one or more peptide immunogen construct described herein is administered to a subject in an initial dose or doses (e.g., in the first 1-3 months) followed by booster doses (e.g., every 1-6 months) administered for a period of time, e.g., for at least or more than 6 months, 9 months, 1 year, 1.5 years, 2 years, 3 years, 5 years, or 10 years. In some embodiments, one or more peptide immunogen construct described herein is administered to a subject 1, 2, or 3 times, resulting in a long-term effect, such that a further booster dose is not administered at least until 6 months later, at least until 1 year later, or at least until 2 years later. In some embodiments, one or more peptide immunogen construct described herein is administered to a subject 1, 2, or 3 times resulting in a lasting improvement (e.g., in muscle strength, muscle performance, or muscle mass) for at least 6 months, 9 months, 1 year, or 2 years.

### Other Exemplary Embodiments I

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Some embodiments are within the scope of the following numbered paragraphs.

1. A peptide immunogen construct comprising a myostatin B cell epitope, a heterologous T cell (Th) epitope, and an optional heterologous spacer, wherein the B cell epitope is covalently linked to the Th epitope directly or through the optional heterologous spacer.

2. The peptide immunogen construct of paragraph 1, wherein the myostatin B cell epitope comprises about 7 to about 30 contiguous amino acids from myostatin or a variant thereof.

3. The peptide immunogen construct of paragraph 1 or 2, wherein the myostatin B cell epitope comprises about 7 to about 30 contiguous amino acids of SEQ ID NO:1 or SEQ ID NO: 2 or a variant thereof.

4. The peptide immunogen construct of paragraph 3, wherein the myostatin B cell epitope comprises a variant of an 7-30 amino acid fragment of SEQ ID NO:1 or SEQ ID NO: 2, and the variant comprises 1, 2, 3, 4, 5, or 6 amino acid substitutions, deletions, or insertions as compared to the corresponding sequence of the 7-30 amino acid fragment of SEQ ID NO: 1 or SEQ ID NO: 2.

5. The peptide immunogen construct of paragraph 4, wherein the myostatin B cell epitope comprises a variant of an 7-30 amino acid fragment of SEQ ID NO:1 or SEQ ID NO: 2, and the variant comprises 1, 2, or 3 amino acid substitutions as compared to the corresponding sequence of the 7-30 amino acid fragment of SEQ ID NO: 1 or SEQ ID NO: 2.

6. The peptide immunogen construct of any one of paragraphs 1 to 5, wherein the myostatin B cell epitope comprises 1, 2, or 3 amino acid substitutions as compared to the sequence of SEQ ID NO: 1 or SEQ ID NO: 2 to facilitate cyclization of the sequence comprising the myostatin B cell epitope.

7. The peptide immunogen construct of any one of paragraphs 1 to 6, wherein the myostatin B cell epitope comprises or consists of an amino acid sequence within loop 1, loop 2, loop 3, or loop 4 of myostatin, or a variant thereof, and optionally comprises or consists of the amino acid sequence of any one of SEQ ID NOs: 3-10, 95-109, 145-164, and 166 or a variant thereof.

8. The peptide immunogen construct of any one of paragraphs 1 to 7, wherein the Th epitope is derived from a pathogenic protein, and optionally comprises or consists of a sequence selected from any one of SEQ ID NOs: 19-47 or a variant thereof.

9. The peptide immunogen construct of any one of paragraphs 1 to 8, wherein the Th epitope comprises or consists of the sequence of SEQ ID NO: 30, 32, or 33.

10. The peptide immunogen construct of any one of paragraphs 1 to 9, wherein the myostatin B cell epitope and the Th epitope are linked to one another by the heterologous spacer.

11. The peptide immunogen construct of paragraph 10, wherein the heterologous spacer comprises or consists of one or more amino acids, which are optionally selected from: Lys-, Gly-, Lys-Lys-Lys-, Lys-Lys-Lys-ε-Lys (SEQ ID NO: 192), (α, ε-N)Lys, ε-N-Lys-Lys-Lys-Lys (SEQ ID NO: 91), and Pro-Pro-Xaa-Pro-Xaa-Pro, wherein Xaa is any amino acid or is aspartic acid (SEQ ID NO: 90).

12. The peptide immunogen construct of any one of paragraphs 1 to 11, wherein: (a) the myostatin B cell epitope is located N-terminal to the Th epitope; (b) the Th epitope is located N-terminal to the myostatin B cell epitope; (c) the myostatin B cell epitope is flanked with a Th epitope on both N-terminal and C-terminal ends; (d) any of (a)-(c) wherein the myostatin B cell epitope(s) is linked to the Th epitope(s) by direct covalent linkage; or (e) any one of (a)-(c) wherein the myostatin B cell epitope(s) is linked to the Th epitope(s) by the heterologous spacer.

13. The peptide immunogen construct of any one of paragraphs 1 to 12, wherein the peptide immunogen construct is of following formula: (Th)ₘ-(A)ₙ-(myostatin B cell epitope) or (myostatin B cell epitope)-(A)ₙ-(Th)ₘ or (Th)ₘ-(A)ₙ-(myostatin B cell epitope)-(A)ₙ-(Th)ₘ wherein Th is the T helper epitope; A is the heterologous spacer, which optionally comprises an amino acid; in is from 1 to about 4; and n is from 0 to about 10, wherein optionally the C-terminus of the peptide immunogen construct comprises α-COOH or α-CONH₂ of an amino acid.

14. The peptide immunogen construct of any one of paragraphs 1 to 13, wherein the peptide immunogen construct comprises or consists of the sequence of any one of SEQ ID NOs: 48-71, 120-134, 167-186 and 188 or a variant thereof.

15. A peptide immunogen construct comprising an activin A B cell epitope, a heterologous T cell (Th) epitope, and an optional heterologous spacer, wherein the B cell epitope is covalently linked to the Th epitope directly or through the optional heterologous spacer.

16. The peptide immunogen construct of paragraph 15, wherein the activin A B cell epitope comprises about 7 to about 30 contiguous amino acids from activin A or a variant thereof.

17. The peptide immunogen construct of paragraph 15 or 16, wherein the activin A B cell epitope comprises about 7 to about 30 contiguous amino acids of SEQ ID NO: 12 or a variant thereof.

18. The peptide immunogen construct of paragraph 17, wherein the activin A B cell epitope comprises a variant of a 7-30 amino acid fragment of SEQ ID NO: 12, and the variant comprises 1, 2, 3, 4, 5, or 6 amino acid substitutions, deletions, or insertions as compared to the corresponding sequence of the 7-30 amino acid fragment of SEQ ID NO: 12.

19. The peptide immunogen construct of paragraph 18, wherein the activin A B cell epitope comprises a variant of a 7-30 amino acid fragment of SEQ ID NO: 12, and the variant comprises 1, 2, or 3 amino acid substitutions as compared to the corresponding sequence of the 7-30 amino acid fragment of SEQ ID NO: 12.

20. The peptide immunogen construct of any one of paragraphs 15 to 19, wherein the activin A B cell epitope comprises 1, 2, or 3 amino acid substitutions as compared to the sequence of SEQ ID NO: 12 to facilitate cyclization of the sequence comprising the activin A B cell epitope.

21. The peptide immunogen construct of any one of paragraphs 15 to 20, wherein the activin A B cell epitope comprises or consists of an amino acid sequence within loop 1, loop 2, loop 3, or loop 4 of activin A, or a variant thereof, and optionally comprises or consists of the amino acid sequence of any one of SEQ ID NOs: 13-18, 110-119, 187, and 189 or a variant thereof.

22. The peptide immunogen construct of any one of paragraphs 15 to 21, wherein the Th epitope is derived from a pathogenic protein, and optionally comprises or consists of a sequence selected from any one of SEQ ID NOs: 19-47 or a variant thereof.

23. The peptide immunogen construct of any one of paragraphs 15 to 22, wherein the Th epitope comprises or consists of the sequence of SEQ ID NO: 30, 32, or 33.

24. The peptide immunogen construct of any one of paragraphs 15 to 23, wherein the activin A B cell epitope and the Th epitope are linked to one another by the heterologous spacer.

25. The peptide immunogen construct of paragraph 24, wherein the heterologous spacer comprises or consists of one or more amino acids, which are optionally selected from: Lys-, Gly-, Lys-Lys-Lys-, Lys-Lys-Lys-ε-Lys (SEQ ID NO: 192), (α, ε-N)Lys, ε-N-Lys-Lys-Lys-Lys (SEQ ID NO: 91), and Pro-Pro-Xaa-Pro-Xaa-Pro, wherein Xaa is any amino acid or is aspartic acid (SEQ ID NO: 90).

26. The peptide immunogen construct of any one of paragraphs 15 to 25, wherein: (a) the activin A B cell epitope is located N-terminal to the Th epitope; (b) the Th epitope is located N-terminal to the activin A B cell epitope; (c) the activin A B cell epitope is flanked with a Th epitope on both N-terminal and C-terminal ends; (d) any of (a)-(c) wherein the activin A B cell epitope(s) is linked to the Th epitope(s) by direct covalent linkage; or (e) any one of (a)-(c) wherein the activin A B cell epitope(s) is linked to the Th epitope(s) by the heterologous spacer.

27. The peptide immunogen construct of any one of paragraphs 15 to 26, wherein the peptide immunogen construct is of following formula: (Th)ₘ-(A)ₙ-(activin A B cell epitope) or (activin A B cell epitope)-(A)ₙ-(Th)ₘ or (Th)ₘ-(A)ₙ-(activin A B cell epitope)-(A)ₙ-(Th)ₘ wherein Th is the T helper epitope; A is the heterologous spacer, which optionally comprises an amino acid; m is from 1 to about 4; and n is from 0 to about 10, wherein optionally the C-terminus of the peptide immunogen construct comprises α-COOH or α-CONH₂ of an amino acid.

28. The peptide immunogen construct of any one of paragraphs 15 to 27, wherein the peptide immunogen construct comprises or consists of the sequence of any one of SEQ ID NOs: 72-89, 135-144, and 190-191 or a variant thereof.

29. A nucleic acid molecule encoding a peptide immunogen construct of any one of paragraphs 1 to 28.

30. A vector comprising the nucleic acid molecule of paragraph 29.

31. An isolated antibody or an epitope-binding fragment thereof that specifically binds to the myostatin B cell epitope of the peptide immunogen construct of any one of paragraphs 1 to 14 or the activin A B cell epitope of the peptide immunogen construct of any one of paragraphs 15 to 28.

32. A composition comprising one or more of: (a) a peptide immunogen construct of any one of paragraphs 1 to 28, a nucleic acid of paragraph 29, a vector of paragraph 30, an isolated antibody of paragraph 31, or a peptide immunogen construct of any one of paragraphs 1 to 14 and a peptide immunogen construct of any one of paragraphs 15 to 28, and (b) a pharmaceutically acceptable carrier, diluent, or adjuvant.

33. The composition of paragraph 32, wherein the composition comprises an adjuvant that is a mineral salt of aluminum, which optionally is selected from Al(OH)₃ and AlPO₄.

34. The composition of paragraph 32 or 33, wherein the composition comprises a CpG oligonucleotide.

35. A method of treating or preventing the development of muscle wasting in a subject, the method comprising administering to the subject an effective amount of an immunotherapy targeting myostatin and/or activin A, wherein the immunotherapy is selected from a peptide immunogen construct of any one of paragraphs 1 to 28, a nucleic acid molecule of paragraph 29, a vector of paragraph 30, an isolated antibody of paragraph 31, a composition of any one of paragraphs 32 to 34, or a combination of any thereof.

36. A method of treating, preventing, reducing, inhibiting, or slowing the development of one or more symptom of a muscle wasting disease in a subject, the method comprising administering to the subject an effective amount of an immunotherapy targeting myostatin and/or activin A, wherein the immunotherapy is selected from a peptide immunogen construct of any one of paragraphs 1 to 28, a nucleic acid molecule of paragraph 29, a vector of paragraph 30, an isolated antibody of paragraph 31, a composition of any one of paragraphs 32 to 34, or a combination thereof.

37. A method of inducing an immune response to myostatin and/or activin A in a subject, the method comprising administering to the subject an effective amount of an immunotherapy targeting myostatin and/or activin A, wherein the immunotherapy is selected from a peptide immunogen construct of any one of paragraphs 1 to 28, a nucleic acid molecule of paragraph 29, a vector of paragraph 30, an isolated antibody of paragraph 31, a composition of any one of paragraphs 32 to 34, or a combination thereof.

38. A method of treating or preventing insulin resistance in a subject, the method comprising administering to the subject an effective amount of an immunotherapy targeting myostatin and/or activin A, wherein the immunotherapy is selected from a peptide immunogen construct of any one of paragraphs 1 to 28, a nucleic acid molecule of paragraph 29, a vector of paragraph 30, an isolated antibody of paragraph 31, or a composition of any one of paragraphs 32 to 34, or a combination thereof.

39. The method of any one of paragraphs 35 to 38, wherein the subject has or is at risk of developing muscle wasting due to an inherited or genetic cause, inflammation, infection, injury, systemic disease, toxin, or other environmental exposure.

40. The method of any one of paragraphs 35 to 39, wherein the subject has an inherited or genetic myopathy, which is optionally selected from muscular dystrophy (e.g., Duchenne Muscular Dystrophy (DMD) and Becker Muscular Dystrophy (BMD), myotonic dystrophy (e.g., DM1 and DM2), facioscapulohumeral muscular dystrophy, oculopharyngeal muscular dystrophy, and limb girdle muscular dystrophy), congenital myopathy (e.g., nemaline myopathy and central core myopathy), metabolic myopathy (e.g., acid maltase or acid alpha-1, 4-glucosidase deficiency (Pompe's disease), glycogen storage disorders 3-11, carnitine deficiency, fatty acid oxidation defects, and carnitine palmitoyl transferase deficiency), and mitochondrial myopathy.

41. The method of any one of paragraphs 35 to 40, wherein the subject has or is at risk of developing an autoimmune or inflammatory myopathy (i.e., myositis), which is optionally selected from dermatomyositis, polymyositis, inclusion body myositis, and juvenile myositis, or another autoimmune disorder (e.g., myasthenia gravis).

42. The method of any one of paragraphs 35 to 41, wherein the subject has or is at risk of developing an infection, which optionally is selected from an infection by a virus (e.g., HIV, influenza virus, and Epstein-Barr virus), a bacterium (e.g., pyomyositis, S. aureaus, and streptococci), a spirochete (e.g., Lyme disease), and a parasite (e.g., trichinosis).

43. The method of any one of paragraphs 35 to 42, wherein the subject has or is at risk of exposure to a toxin, which optionally is selected from a medication (e.g., a cholesterol-lowering medication (e.g., a statin), propofol, amiodaron, colchicine, chloroquine, an antiviral, a protease inhibitor, omeprazole, and tryptophan), alcohol, and toluene.

44. The method of any one of paragraphs 35 to 43, wherein the subject has or is at risk of developing a systemic disease, which optionally is selected from an endocrine disorder (e.g., thyroid, parathyroid, pituitary, and adrenal disorder), systemic inflammatory disease (e.g., systemic lupus erythematosus, rheumatoid arthritis, scleroderma, Sjogren's syndrome, mixed connective disease, and sarcoidosis), electrolyte imbalance, critical illness myopathy, and amyloid myopathy.

45. The method of any one of paragraphs 35 to 44, wherein the subject has or is at risk of developing a neurological disease or condition, which optionally is selected from amyotrophic lateral sclerosis (ALS), multiple sclerosis (MS), spinal muscular atrophy (SMA), Guillain-Barre syndrome, carpel tunnel syndrome, Charcot-Marie-Tooth disease, Parkinson's disease, Lewy body disease, polio, spinal cord injury, peripheral nerve injury, stroke, and nerve damage caused by diabetes, toxins, or alcohol.

46. The method of any one of paragraphs 35 to 45, wherein the subject is or is expected to be present in an environment with decreased or no gravity, e.g., during a space flight or being at a space station or the like.

47. The method of any one of paragraphs 35 to 46, wherein the subject has or experiences prolonged inactivity (e.g., is bedridden, has a seated job or a sedentary lifestyle, or cannot move limbs due to stroke or other brain disease), burn, long-term corticosteroid therapy, malnutrition, anorexia, cachexia, osteoarthritis, advanced age, cancer, chronic kidney disease, heart failure, or chronic obstructive pulmonary disease.

48. The method of any one of paragraphs 35 to 47, wherein the subject has or is at risk of developing one or more symptom selected from muscle loss, movement issues, balance problems, trouble walking, trouble using arms or legs, contractures, muscle rigidity, muscle twitching, fasciculations, muscle cramps, numbness, tingling, painful sensations, breathing problems, curved spine, heart problems, swallowing problems (which may lead to, e.g., nutritional problems and aspiration pneumonia), reduced muscle mass (e.g., in general or in one limb), weakness (e.g., in general or in one limb), pain, discomfort, one arm or leg being smaller than the other, weakness in one arm or leg, numbness or tingling in an arm or leg, trouble walking, balance problems, facia weakness, gradual memory loss, reduced muscle strength, impaired ability to perform physical activities, decrease in muscle size, cramps, stiffness, spasms, fatigue, motor delay, respiratory impairment, and bulbar muscle dysfunction.

49. The method of any one of paragraphs 35 to 48, wherein the subject has or is at risk of developing insulin resistance, type II diabetes, elevated A1C levels, pre-diabetes, metabolic syndrome, hypertension, hyperglycemia, hypercholesterolemia, high triglycerides, overweight, or obesity.

### Other Exemplary Embodiments II

Additional contemplated embodiments of the invention are set forth in the following numbered paragraphs.

1. A peptide immunogen construct comprising a myostatin B cell epitope, a heterologous T cell (Th) epitope, and an optional heterologous spacer, wherein the B cell epitope is covalently linked to the Th epitope directly or through the optional heterologous spacer; wherein the myostatin B cell epitope is any one of the B cell epitopes set forth in Table 1 (where this disclosure is meant to individually and specifically single out each of the myostatin B cell epitopes set forth in Table 1), optionally wherein the myostatin B cell epitope is cyclized or is capable of cyclization; further optionally where the myostatin B cell epitope is acetylated on the N-terminus or cyclized using the N-terminus amino group and/or has an amide on the C-terminus.

2. The peptide immunogen construct of paragraph 1, wherein the Th epitope is selected from any one of SEQ ID NOs: 19-47, optionally wherein the Th epitope is of SEQ ID NO: 30, 32, or 33; further optionally wherein the Th epitope is of SEQ ID NO: 32 (UBITh1).

3. The peptide immunogen construct of paragraph 2, wherein the myostatin B cell epitope and the Th epitope are linked to one another by the heterologous spacer, wherein the heterologous spacer is, or comprises, Lys-, Gly-, Lys-Lys-Lys-, Lys-Lys-Lys-ε-Lys (SEQ ID NO: 192), (α, ε-N)Lys, ε-N-Lys-Lys-Lys-Lys (SEQ ID NO: 91), or Pro-Pro-Xaa-Pro-Xaa-Pro, wherein Xaa is any amino acid or is aspartic acid (SEQ ID NO: 90), optionally wherein the heterologous spacer comprises or consists of 3 or 4 lysines (optionally including an epsilon lysine), or the heterologous spacer is Lys-Lys-ε-Lys (SEQ ID NO: 192), (α, ε-N)Lys, or ε-N-Lys-Lys-Lys-Lys (SEQ ID NO: 91).

4. The peptide immunogen construct of any one of paragraphs 1-3, wherein the peptide immunogen construct is of the following formula:

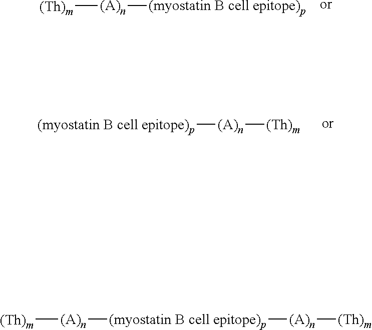

wherein
Th is the T helper epitope;
A is the heterologous spacer, which optionally comprises an amino acid;
m is from 1 to about 4;
p is from 1 to about 4;
and
n is from 0 to about 10, wherein optionally the C-terminus of the peptide immunogen construct comprises α-COOH or α-CONH₂ of an amino acid; optionally wherein m is 1, p is 1 or 2, and n is 1.

5. The peptide immunogen construct of paragraph 1, wherein the peptide immunogen construct comprises or consists of the sequence of any one of SEQ ID NOs: 48-71, 120-134, 167-186 and 188 or a variant thereof; optionally wherein the peptide immunogen construct comprises or consists of the sequence of any one of: SEQ ID NO: 54, 55, 56, 122, 123, 126, 130, 168, 169, 173, 174, 175, 176, 177, 178, 179, 181, 183, 185, or 186.

6. A myostatin peptide immunogen construct comprising or consisting of a sequence set forth in Table 4 (where this disclosure is meant to individually and specifically single out each of the myostatin peptide immunogen constructs set forth in Table 4).

7. A peptide immunogen construct comprising an activin A B cell epitope, a heterologous T cell (Th) epitope, and an optional heterologous spacer, wherein the B cell epitope is covalently linked to the Th epitope directly or through the optional heterologous spacer, wherein the activin A B cell epitope is any one of the B cell epitopes set forth in Table 2 (where this disclosure is meant to individually and specifically single out each of the activin A B cell epitopes set forth in Table 2), optionally wherein the activin A B cell epitope is cyclized or is capable of cyclization; further optionally the activin A B cell epitope is acetylated on the N-terminus or cyclized using the N-terminus amino group and/or has an amide on the C-terminus.

8. The peptide immunogen construct of paragraph 7, wherein the Th epitope is selected from any one of SEQ ID NOs: 19-47, optionally wherein the Th epitope comprises or consists of the sequence of SEQ ID NO: 30, 32, or 33; further optionally wherein the Th epitope is of SEQ ID NO: 32 (UBITh1).

9. The peptide immunogen construct of paragraph 8, wherein the activin A B cell epitope and the Th epitope are linked to one another by the heterologous spacer, wherein the heterologous spacer is, or comprises, Lys-, Gly-, Lys-Lys-Lys-, Lys-Lys-Lys-ε-Lys (SEQ ID NO: 192), (α, ε-N)Lys, ε-N-Lys-Lys-Lys-Lys (SEQ ID NO: 91), or Pro-Pro-Xaa-Pro-Xaa-Pro, wherein Xaa is any amino acid or is aspartic acid (SEQ ID NO: 90); optionally wherein the heterologous spacer comprises or consists of 3 or 4 lysines (optionally including an epsilon lysine), or the heterologous spacer is Lys-Lys-Lys-ε-Lys (SEQ ID NO: 1.92), (α, ε-N)Lys, or ε-N-Lys-Lys-Lys-Lys (SEQ ID NO: 91).

10. The peptide immunogen construct of any one of paragraphs 7-9, wherein the peptide immunogen construct is of the following formula:

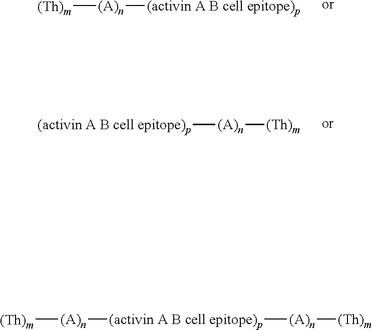

wherein
Th is the T helper epitope;
A is the heterologous spacer, which optionally comprises an amino acid;
m is from 1 to about 4;
p is from 1 to about 4;
and
n is from 0 to about 10, wherein optionally the C-terminus of the peptide immunogen construct comprises α-COOH or α-CONH₂ of an amino acid; optionally m is 1, p is 1 or 2, and n is 1.

11. The peptide immunogen construct of paragraph 7, wherein the peptide immunogen construct comprises or consists of the sequence of any one of SEQ ID NOs: 72-89, 135-144, and 190-191 or a variant thereof; optionally wherein the peptide immunogen construct comprises or consists of the sequence of any one of: SEQ ID NOs: 75, 76, 78, 79, 85, 136, 138, 139, 140, 141, 142, 143, 144, or 190; optionally wherein the construct comprises or consists of SEQ ID NOs: 75, 76, 78, 79, 85, 136, 141, 143, 144 or 190.

12. An activin A peptide immunogen construct comprising or consisting of a sequence set forth in Table 5 (where this disclosure is meant to individually and specifically single out each of the myostatin peptide immunogen constructs set forth in Table 5).

13. A peptide immunogen construct comprising:
(i) two or more B cell epitopes selected from:
   a myostatin B cell epitope set forth in Table 1 (where this disclosure is meant to individually and specifically single out each of the myostatin B cell epitopes set forth in Table 1), optionally wherein the B cell epitope is cyclized, and
   an activin A B cell epitope set forth in Table 2 (where this disclosure is meant to individually and specifically single out each of the myostatin B cell epitopes set forth in Table 2), optionally wherein the B cell epitope is cyclized;
   optionally wherein the two or more B cell epitopes are two to four B cell epitopes, further optionally wherein the two or more B cell epitopes are two or three B cell epitopes
(ii) a heterologous T cell (Th) epitope, wherein the Th epitope is selected from any one of SEQ ID NOs: 19-47, optionally wherein the Th epitope is of SEQ ID NO: 30, 32, or 33; further optionally wherein the Th epitope is of SEQ ID NO: 32 (UBITh1); and
(iii) optionally, one or more heterologous spacers and/or linkers,
wherein the myostatin and activin A B cell epitopes are covalently linked to the Th epitope directly or through the one or more heterologous spacers and/or linkers.

14. The peptide immunogen construct of paragraph 13, wherein the myostatin B cell epitope and the Th epitope are linked to one another by the heterologous spacer, wherein the heterologous spacer is, or comprises, Lys-, Gly-, Lys-Lys-Lys-, Lys-Lys-Lys-ε-Lys (SEQ ID NO: 192), (α, ε-N)Lys, ε-N-Lys-Lys-Lys-Lys (SEQ ID NO: 91), or Pro-Pro-Xaa-Pro-Xaa-Pro, wherein Xaa is any amino acid or is aspartic acid (SEQ ID NO: 90), optionally wherein the heterologous spacer comprises or consists of 3 or 4 lysines (optionally including an epsilon lysine), or the heterologous spacer is Lys-Lys-ε-Lys (SEQ ID NO: 192), (α, ε-N)Lys, or ε-N-Lys-Lys-Lys-Lys (SEQ ID NO: 91).

15. The peptide immunogen construct of paragraph 13 or 14, wherein the two or more B cell epitopes are two or more of the myostatin B cell epitopes, optionally wherein the two or more B cell epitopes do not comprise an activin A B cell epitope.

16. The peptide immunogen construct of paragraph 13 or 14, wherein the two or more B cell epitopes are two or more of the activin A B cell epitopes, optionally wherein the two or more B cell epitopes do not comprise a myostatin B cell epitope.

17. The peptide immunogen construct of paragraph 13 or 14, wherein the two or more B cell epitopes comprise at least one myostatin B cell epitope and at least one activin A B cell epitope; optionally wherein the two or more B cell epitopes comprise or consist of one myostatin B cell epitope and one activin A B cell epitope.

18. The peptide immunogen construct of any one of paragraphs 13-17, wherein the one or more heterologous spacers and/or linkers comprise a branched amino acid, optionally wherein the branched amino acid is (i) a diamino acid and the B cell epitopes are attached to the N-terminus of the Th epitope, optionally wherein the diamino acid is lysine, or (ii) a dicarboxylic acid and the B cell epitopes are attached to the C-terminus of the Th epitope, optionally wherein the dicarboxylic acid is glutamic acid.

19. The peptide immunogen construct of any one of paragraph 18, wherein the branched amino acid is linked to each of the B cell epitopes by a linker, optionally wherein the linker is an amino acid peg derivative; optionally wherein the peg derivative is 8-amino-3,6-dioxaoctanoic acid or 2-[2-(2-aminoethoxy)ethoxy]acetic acid (“AeeA”); further optionally wherein the branched amino acid is covalently linked to the Th epitope by a heterologous spacer, optionally wherein the heterologous spacer is a polylysine spacer, optionally comprising or consisting of 3 or 4 lysines, or wherein the heterologous spacer is Lys-Lys-Lys-ε-Lys (SEQ ID NO: 192), (α, ε-N)Lys, or ε-N-Lys-Lys-Lys-Lys (SEQ ID NO: 91).

20. The peptide immunogen construct of paragraph 13, which comprises or consists of

(SEQ ID NO: 165)
Ac-STVINHYRMRGHSPFANLKSSAeeA(Ac-QKYPHTHLVHQANPRGSA
GPAeeA)K-KKKrK-Ubith1.

21. A nucleic acid molecule encoding a peptide immunogen construct of any one of paragraphs 1-20.

22. A vector comprising the nucleic acid molecule of paragraph 21.

23. An isolated antibody or an epitope-binding fragment thereof that specifically binds to and/or elicited by the myostatin B cell epitope identified in Table 1, and/or that specifically binds to and/or elicited by the activin A B cell epitope identified in Table 2.

24. A pharmaceutical composition comprising one or more of: (a) one or more peptide immunogen constructs of any one of paragraphs 1-20, a nucleic acid of paragraph 21, a vector of paragraph 22, an isolated antibody of paragraph 23, and (b) a pharmaceutically acceptable carrier, diluent, or adjuvant.

25. The pharmaceutical composition of paragraph 24, which comprises a CpG oligonucleotide, optionally a GpG1 oligonucleotide.

26. The pharmaceutical composition of paragraph 25, which comprises an adjuvant, optionally wherein the adjuvant is a mineral salt of aluminum, which optionally is selected from Al(OH)₃ and AlPO₄.

27. A method of inducing an immune response to myostatin and/or activin A in a subject, the method comprising administering to the subject an effective amount of an immunotherapy targeting myostatin and/or activin A, wherein the immunotherapy is selected from a peptide immunogen construct of any one of paragraphs 1-20, a nucleic acid molecule of paragraph 21, a vector of paragraph 22, an isolated antibody of paragraph 23, or a pharmaceutical composition of any one of paragraphs 24-26.

28. A method of treating, preventing, reducing, inhibiting, or slowing the development of muscle wasting or a symptom thereof in a subject, the method comprising administering to the subject an effective amount of an immunotherapy targeting myostatin and/or activin A, wherein the immunotherapy is selected from a peptide immunogen construct of any one of paragraphs 1-20, a nucleic acid molecule of paragraph 21, a vector of paragraph 22, an isolated antibody of paragraph 23, or a pharmaceutical composition of any one of paragraphs 24-26.

29. The method of paragraph 28, wherein the subject has or is at risk of developing muscle wasting due to an inherited or genetic cause, inflammation, an autoimmune disorder, infection, injury, systemic disease, a neurological disease, a toxin, an environmental exposure, prolonged inactivity, malnutrition, anorexia, cachexia, osteoarthritis, advanced age, cancer, or chronic disease.

30. A method of treating, preventing, reducing, inhibiting, or slowing the development of sarcopenia or a symptom thereof in a subject (e.g., where the subject is at least 60, 65 or 70 years of age), the method comprising administering to the subject an effective amount of an immunotherapy targeting myostatin and/or activin A, wherein the immunotherapy is selected from a peptide immunogen construct of any one of paragraphs 1-20, a nucleic acid molecule of paragraph 21, a vector of paragraph 22, an isolated antibody of paragraph 23, or a pharmaceutical composition of any one of paragraphs 24-26.

31. A method of treating, preventing, reducing, inhibiting, or slowing the development of cachexia or a symptom thereof in a subject, the method comprising administering to the subject an effective amount of an immunotherapy targeting myostatin and/or activin A, wherein the immunotherapy is selected from a peptide immunogen construct of any one of paragraphs 1-20, a nucleic acid molecule of paragraph 21, a vector of paragraph 22, an isolated antibody of paragraph 23, or a pharmaceutical composition of any one of paragraphs 24-26.

32. A method of treating or preventing insulin resistance in a subject, the method comprising administering to the subject an effective amount of an immunotherapy targeting myostatin and/or activin A, wherein the immunotherapy is selected from a peptide immunogen construct of any one of paragraphs 1-20, a nucleic acid molecule of paragraph 21, a vector of paragraph 22, an isolated antibody of paragraph 23, or a pharmaceutical composition of any one of paragraphs 24-26.

33. A method of increasing lean mass and/or reducing fat mass in a subject, the method comprising administering to the subject an effective amount of an immunotherapy targeting myostatin and/or activin A, wherein the immunotherapy is selected from a peptide immunogen construct of any one of paragraphs 1-20, a nucleic acid molecule of paragraph 21, a vector of paragraph 22, an isolated antibody of paragraph 23, or a pharmaceutical composition of any one of paragraphs 24-26.

34. The method of paragraph 32 or 33, wherein the subject has or is at risk of developing insulin resistance, type II diabetes, elevated A1C levels, pre-diabetes, metabolic syndrome, hypertension, hyperglycemia, hypercholesterolemia, high triglycerides, overweight body mass, or obesity.

35. A method of treating or preventing muscular or musculoskeletal injury (e.g., muscle tear or muscle strain) in a subject, the method comprising administering to the subject an effective amount of an immunotherapy targeting myostatin and/or activin A, wherein the immunotherapy is selected from a peptide immunogen construct of any one of paragraphs 1-20, a nucleic acid molecule of paragraph 21, a vector of paragraph 22, an isolated antibody of paragraph 23, or a pharmaceutical composition of any one of paragraphs 24-26.

36. The method of any one of paragraphs 27-35, wherein the subject is a human.

37. A pharmaceutical composition comprising a myostatin peptide immunogen construct of any one of paragraphs 1 to 6 and an activin A peptide immunogen construct of any one of paragraphs 7 to 12, optionally comprising adjuvants or excipients specified in any one of paragraphs 24-26.

38. A kit comprising a myostatin peptide immunogen construct of any one of paragraphs 1 to 6 and an activin A peptide immunogen construct of any one of paragraphs 7 to 12, optionally comprising adjuvants or excipients specified in any one of paragraphs 24-26.

39. A method of treating any disease or condition described herein (e.g., muscle wasting or sarcopenia) comprising administering to the subject the pharmaceutical composition of paragraph 37, optionally the method is as set forth in any one of paragraphs 27-36.

40. A composition comprising: (i) a means for eliciting an immune response to myostatin; (ii) a means for eliciting an immune response to activin A; and optionally a pharmaceutically acceptable adjuvant or excipient.

41. A method of treating any disease or condition described herein (e.g., muscle wasting or sarcopenia) comprising administering to the subject the composition of paragraph 40, optionally the method is as set forth in any one of paragraphs 27-36.

The following examples illustrate certain features and aspects of the disclosure and are not to be considered as limiting of the scope of the disclosure in any way.

### EXAMPLES

### Example 1

Myostatin is a secreted growth differentiation factor that is a member of the TGFβ protein family and is a negative regulator of muscle growth. It is synthesized as a 375 amino acid precursor, which includes an N-terminal signal sequence for secretion. The precursor protein is cleaved by a serine protease at an RSRR site to generate a latency-associated peptide (LAP) and mature myostatin, with the latter being characterized by nine conserved cysteine residues that play a role in eventual homo-dimerization. Mature myostatin is comprised of amino acids 267-375 of the proprotein and is characterized by intrachain disulfide bonds between the following sets of amino acids: 281-340, 309-372, and 313-374. The three-dimensional structure of myostatin is characterized by four loops (loops 1-4). We have designed peptide immunogens corresponding to certain myostatin loop sequences. In some examples, the peptides were designed to include certain amino acid substitutions that facilitate cyclization of the peptides, resulting in structures that mimic those of the sequences when present in the context of an intact loop. We have found that the designed peptide immunogens, present in the context of constructs that also include heterologous T cell epitopes, are immunogenic in animal models. The induced antibodies have neutralizing effects in cell culture studies, and the peptide immunogens can provide improvements in features of muscle wasting in an animal model system. Additional information about these studies is provided below.

### Immunogenicity

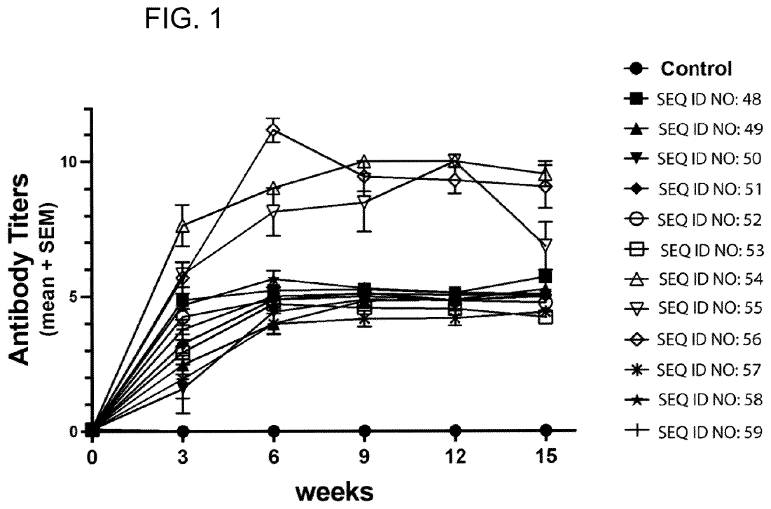

The myostatin peptide immunogen constructs listed in FIG. 1 were injected IM into Guinea pigs at 400 ug peptide in montanide (ISA-51) on week 0, and at 100 ug peptide in montanide on weeks 3, 6, 9 and 12. Serum samples obtained at 6, 9, 12, and 15 weeks-post immunization (wpi) were diluted as indicated in the figure and OD 450 readings were obtained. The results show that the peptide immunogen constructs are immunogenic as compared to placebo and pre-immune sera, with SEQ ID NOs: 54-56 showing the highest activity.

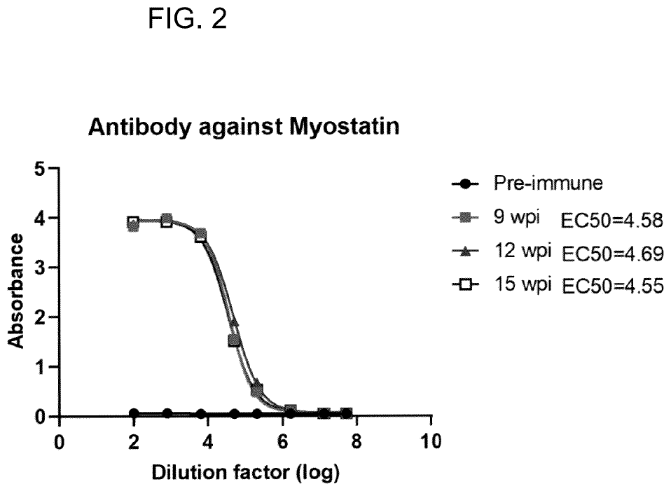

Four Guinea pigs were vaccinated with a peptide of SEQ ID NO: 55 at 400 μg per injection, IM in monatide ISA-51, at 0, 3, 6, 9, and 12 wpi. Sera were collected on weeks 9, 12, and 15 post-immunization. Pooled sera were analyzed on plates coated with full-length myostatin protein. FIG. 2 shows that the immunizations resulted in the production of antibodies that recognize myostatin, as compared to pre-immune sera.

### Neutralization—HEK293 Cells

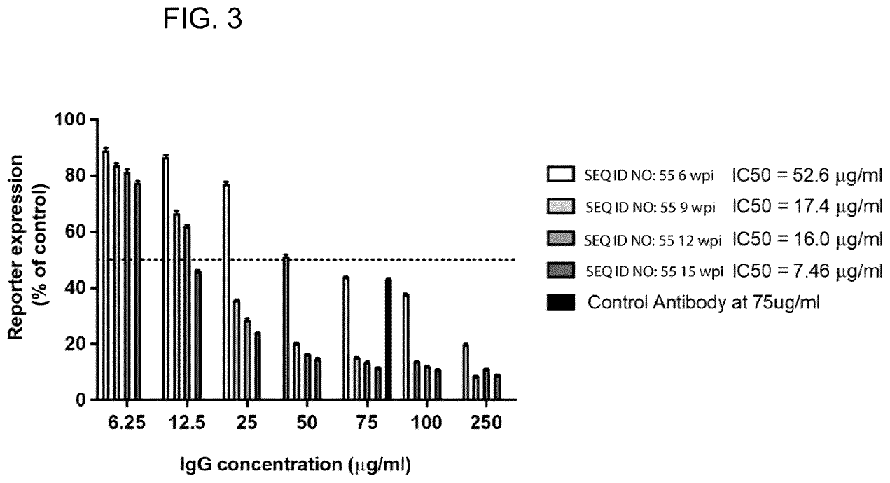

Peptide (SEQ ID NO: 55)-induced antibodies were tested in a neutralization assay in HEK293 cells. Sera from immunized Guinea pigs were collected on weeks 6, 9, 12, and 15 wpi. IgG fractions were isolated and tested for in vitro activity in a reporter assay. Myostatin can bind to TGFβ receptor, composed of two TβR-I and two TβR-II, activate downstream smad pathway, and then induce expression of a reporter protein, SEAP. Neutralizing effects of peptide (SEQ ID NO: 55)-induced IgGs were evaluated in this assay along with a control monoclonal antibody against myostatin. The results are shown in FIG. 3, which shows concentration and time post-immunization effects of peptide (SEQ ID NO: 55) immunization on neutralization.

Methods: Human TGFβ SEAP reporter HEK293 cells (InvivoGen) were grown following the manufacturer's instructions. All cells were cultured in 5% CO₂ at 37° C., and the medium was replaced every 3 days. HEK-blue TGFβ cells allow the detection of bioactive TGFβ by monitoring the activation of the TGFβ/Smad pathway. They were generated by stable transfection of HEK-293 cells with the human TGFBRI, Smad 3, and Smad 4 genes. For neutralization, IgGs diluted with 2% FBS in DMEM mixed with 250 ng/mL myostatin and neutralized for 1 hour. Removed medium of HEK-blue TGFβ and added 100 uL mixed solution in each well of 96 well plates. Incubated the plate at 37° C. in a CO₂ incubator for 16 hours. Prepared 160 μL of resuspended QUANTI-Blue per well and added 40 μL of induced cells supernatant. After incubating for 1 hour, the determination of SEAP levels by using a spectrophotometer at 620-655 nm.

### Neutralization—C2C12 Cells

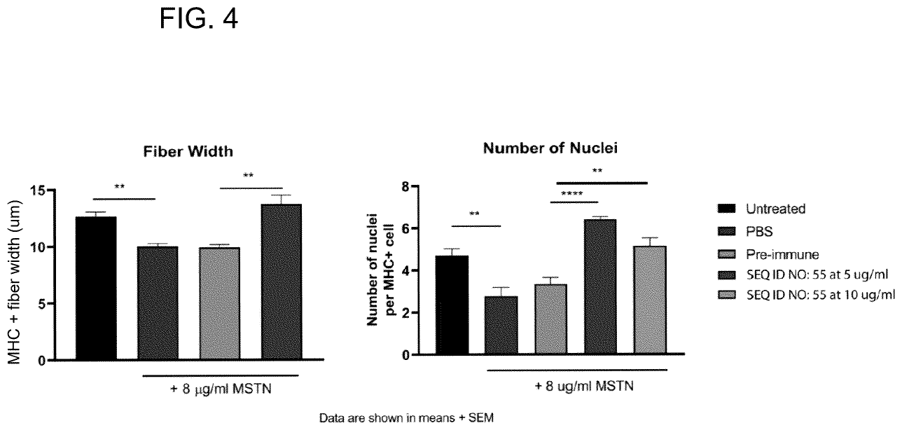

Myostatin inhibits muscle cell differentiation in C2C12 cells. Peptide (SEQ ID NO: 55)-induced antibodies were evaluated in myostatin-treated C2C12 cells. Muscle cell differentiation was determined by fiber width and number of nuclei per cell. The results are shown in FIG. 4. Treatment with peptide (SEQ ID NO: 55) (5 ug/mL) countered the effects of 8 μg/mL myostatin on fiber width. Treatment with peptide (SEQ ID NO: 55) (5 and 10 μg/mL) also countered the effects of 8 μg/mL myostatin on number of nuclei per MHC+ cell.

Methods: C2C12 mouse skeletal muscle cell line obtained from ATCC were grown in high-glucose Dulbecco's modified Eagle's medium (DMEM, 12800-017, Thermo Fisher) supplemented with 10% fetal bovine serum (10437-208, Thermo Fisher) and penicillin-streptomycin 100 IU/mL in 5% CO₂ at 37° C. The growth medium was changed every 3 days. C2C12 cells that had grown to approximately 80% confluence in culture flasks were trypsinized and seeded into culture plates to allow incubation in DMEM containing 10% FBS until reaching about 90% confluence. To induce myoblast differentiation, C2C12 cells were grown in DMEM supplemented with 2% horse serum (16050-122, Thermo Fisher) for different stages14. After the medium was replaced with DMEM containing 2% horse serum (HS), the cells were induced to differentiate into myotubes until fusion of more than 90% cells into myotubes.

C2C12 myoblasts were seeded onto Nunc Thermanox coverslips or 96 well-plates at a density of 20,000 cells/cm². After a 16-hour attachment period, DMEM with 2% HS was added to culture. The cells were incubated at 37° C. in 5% CO₂ for 72 hours, after which the media was replaced with fresh media for a further 72 hours. IgGs were pre-incubated with 8 g/mL myostatin for 2 hours and then added to the cells at day 6 for a further 72 hours.

### In Vivo Studies

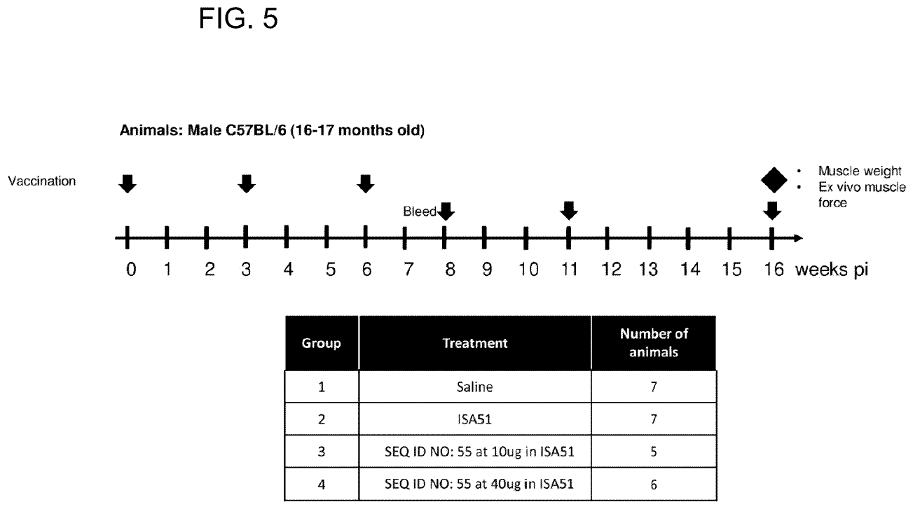

In vivo effects of peptide (SEQ ID NO: 55) immunization of mice were analyzed by testing for antibody titers, as well as effects on body weight and muscle force. The treatment regimen is described below and illustrated in FIG. 5.

Animals. Naïve male C57BL/6 mice (15-16 months old) were obtained from National Laboratory Animal Center (Taiwan). Mice were kept in a humidity and temperature-controlled vivarium on a 12-hour light/dark cycle and were allowed access to food ad libitum. Mice were housed in middle-sized cage and each cage housed up to 8 littermates. Mice were at allowed to acclimate for at least 3 days and were 16-17 months old at study initiation. All animal care and use followed the Institutional Animal Care and Use Committee guidelines set forth by the United Biomedical, Inc. Asia (UBIA). It was noted that animal #33 (no bedding), #22 (vaccinated to blood vessel), #24, and #37 were found dead on 1, 4, 11, and 16 wpi, respectively.

Vaccination. Mice were first pseudo-randomly assigned to different groups adjusted for matching average body weights. The mean of body weight of each group was approximate. After grouping, mice were immunized with 0.2 ml of high dose of peptide (SEQ ID NO: 55) (200 μg/ml), low dose of peptide (SEQ ID NO: 55) (50 μg/ml), adjuvant (ISA51), or saline on thigh (0.1 ml/thigh). Mice received vaccination at 0, 3, and 6 wpi.

Sera collection. Whole blood samples were collected from facial vein by lancet every 2-3 weeks. Blood was allowed to clot by keeping at room temperature for at least 30 minutes. Sera were collected from the supernatants after centrifugation at 5,000 g for 10 minutes at 22° C. and stored at −80° C. until use.

### Antibody Titers

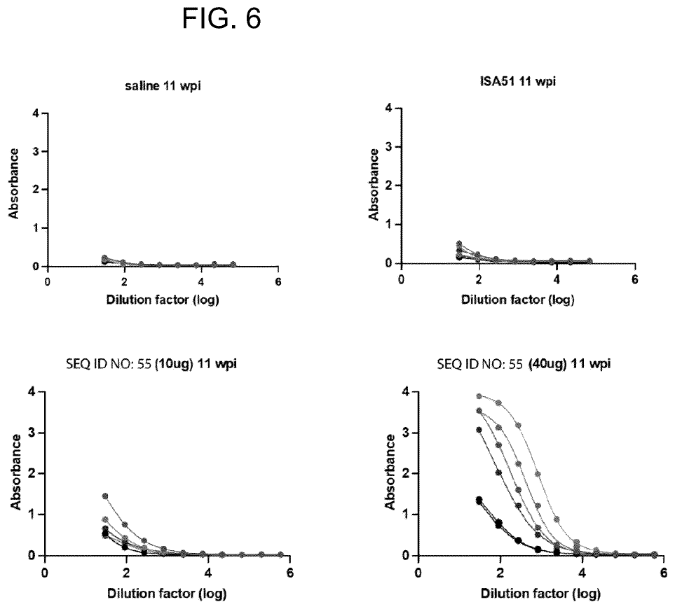

The results of antibody titer analysis at week 11 are shown in FIG. 6. The results show the presence of antibodies in sera in tested in peptide-coated wells from mice immunized with peptide (SEQ ID NO: 55) (10 μg or 40 μg), as compared to saline and adjuvant (ISA-51) only control.

Methods: Pre-immune and immune sera from animals were collected according to experimental vaccination protocols and heated at 56° C. for 30 minutes to inactivate serum complement factors. The peptide-coated wells were incubated with 250 μL of 3% by weight of gelatin in PBS in 37° C. for 1 hour to block non-specific protein binding sites, followed by three washes with PBS containing 0.05% by volume of TWEEN® 20 and dried. 100 μL of samples were added to each of the wells and allowed to react for 60 minutes at 37° C. The wells were then washed six times with 0.05% by volume TWEEN® 20 in PBS to remove unbound antibodies. Horseradish peroxidase (HRP)-conjugated species-specific goat anti-IgG was used as a labeled tracer to bind with the antibody/peptide antigen complex formed in positive wells. One hundred microliters of the peroxidase-labeled goat anti-IgG, at a pre-titered optimal dilution and in 1% by volume normal goat serum with 0.05% by volume TWEEN® 20 in PBS, was added to each well and incubated at 37° C. for another 30 minutes. The wells were washed six times with 0.05% by volume TWEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1M H2SO4 and absorbance at 450 nm (A450) determined. For the determination of antibody titers of the vaccinated animals that received the various myostatin derived peptide immunogens, 10-fold serial dilutions of sera from 1:100 to 1:10,000 were tested, and the titer of a tested serum, expressed as Log 10, was calculated by linear regression analysis of the A450 with the cutoff A450 set at 0.5. Serially diluted sera were tested and positive titers were expressed as Log 10 of the reciprocal dilution.

### Body Weight

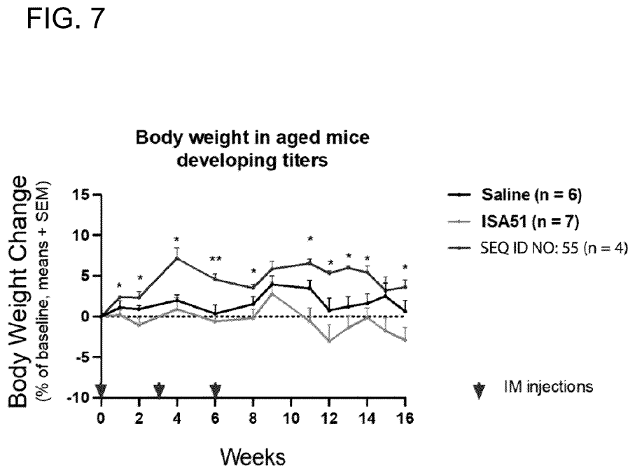

The results of body weight analysis are shown in FIG. 7. The results show that the body weights of mice vaccinated with peptide (SEQ ID NO: 55) were higher than those of mice in the saline and adjuvant (ISA-51) only treated controls.

Body weights were measured on a weekly or bi-weekly basis. Only control mice (administered saline or adjuvant) and mice developing high antibody titers after immunization with peptide (SEQ ID NO: 55) are included in the analysis (4 mice in the peptide (SEQ ID NO: 55) 40 mg/dose group). Body weights are presented as mean percentage change from baseline+SEM. A two-way ANOVA indicated significant treatment x time interaction (F_{24,167}=2.703; p<0.001), a significant group effect (F_{2,14}=5.738; p<0.05) and a significant time effect (F_{3.679,51.20}=7.526; p<0.001). * p<0.05, ** p<0.01 versus ISA51-treated mice.

### Muscle Force

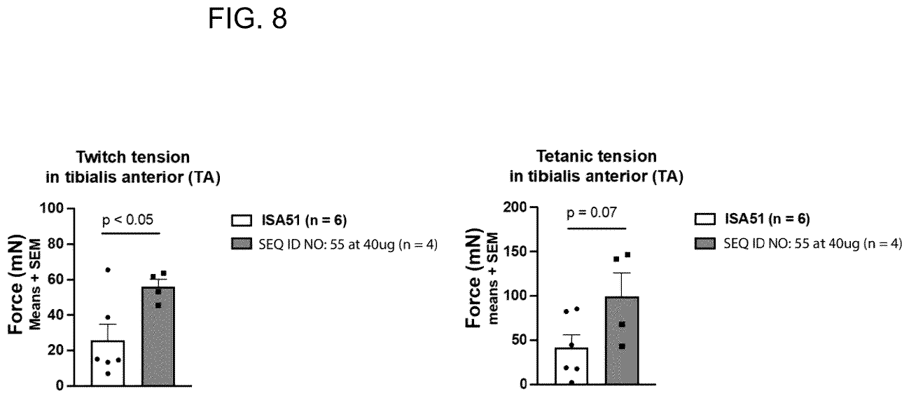

The results of ex vivo muscle force analysis are shown in FIG. 8. The results show increased twitch tension and tetanic tension in tibialis anterior of muscles from mice immunized with peptide (SEQ ID NO: 55), as compared to adjuvant (ISA-51) only treated mice.

Muscle extraction. At completion of the 16-week in-life period, mice were sacrificed by cervical dislocation and their legs were ablated. The skin was removed from the leg. The fascia covering the tibialis anterior (TA) muscle was gently removed. After that, a fishhook was tied on the distal TA tendon by inflexible surgical suture. The distal TA tendon was then cut and used to peel off the TA muscle. Another fishhook was tied on the proximal TA tendon. Lastly, the TA muscle was removed by cutting the proximal tendon. Once removing the TA muscle, the extensor digitorum longus (EDL) muscle was exposed. The EDL muscle was extracted the same way as the TA muscle. To remove the GA muscle, Achilles tendon was cut and used to peel off the gastrocnemius (GA) muscle. As the GA muscle was lifted, the SOL muscle becomes apparent on the underside of the GA muscle. The proximal soleus muscles (SOL) tendon was cut and used to pull the SOL muscle away from the GA muscle. After the whole SOL muscle was free from the GA muscle, cutting the distal SOL tendon to extract the SOL muscle. Finally, the GA muscle was removed by cutting the proximal GA tendon. All muscles were extracted within 1 hour after sacrifice. Muscles were either preserved in Krebs-Henseleit (KH) buffer (118 mM NaCl, 4.7 mM KCl, 1.2 mM MgSO₄, 1.25 mM CaCl₂), 1.2 mM KH₂PO₄, 25 mM NaHCO₃, 11 mM glucose, pH 7.4) for ex vivo force measurement, or transferred to 4% paraformaldehyde (PFA) for long-term storage.

Ex vivo force measurement. Muscles were immersed in KH buffer at 25° C. in 4-channel tissue bath system (SINGA #MB04II, Taiwan, accompanied with Xctionview 2.0 software) with air bubbling (O₂:CO₂=95%:5%) for 15 minutes prior to force measurement. For twitch and tetanic tension measurement, muscles were tightly secured at two hooks, and the length was measured. Maximal twitch tension (MTT) was determined by stimulating muscles with a range of voltages from 5 to 30 V. Each stimulation lasted 0.5 millisecond and muscles were allowed to rest for at least 30 seconds between each stimulation. After MTT was determined, muscles were allowed to rest for at least 3 minutes before further stimulation. For twitch tension (Pt), a supramaximal voltage (20% above MTT) was applied to muscles for around 0.5 millisecond, and contractile force was recorded right after stimulation. For tetanic tension (Po), a supramaximal voltage (20% above MTT) at 150 Hz (totally 300 ms) was applied to muscles, contractile force was measured.

### Example 2

Activin A is a member of the activin family of proteins, which in turn are members of the TGF-β superfamily. Like myostatin, activin A activates the Smad2/3 transcription pathway, which, by limiting protein synthesis and increasing protein degradation, results in a reduction in muscle mass. Activin A is synthesized as a 426 amino acid precursor, which includes an N-terminal signal sequence for secretion. The precursor protein is cleaved by a protease at an RRRR site to generate mature Activin A. Mature activin A is comprised of amino acids 311-426 of the proprotein and is characterized by intrachain disulfide bonds. The three-dimensional structure of activin A is characterized by four loops (loops 1-4). We have designed peptide immunogens corresponding to certain activin A loop sequences. In some examples, the peptides were designed to include certain amino acid substitutions that facilitate cyclization of the peptides, resulting in structures that mimic those of the sequences when present in the context of an intact loop. We have found that the designed peptide immunogens, present in the context of constructs that also include heterologous T cell epitopes, are immunogenic an animal model. The induced antibodies have neutralizing effects in cell culture studies. Additional information about these studies is provided below.

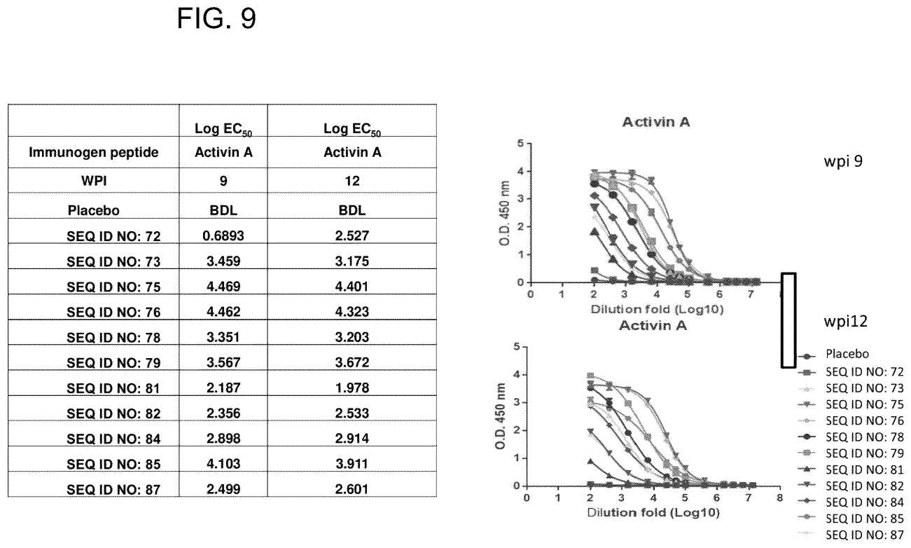

The activin A peptide immunogen constructs listed in FIG. 9 were injected IM into Guinea pigs (400 ug in montanide ISA-51). Serum samples obtained at 9 and 12 weeks-post immunization (wpi) were diluted as indicated in the figure and OD 450 readings were obtained. The results show that the peptide immunogen constructs are immunogenic as compared to placebo, with SEQ ID NOs: 73, 75, 76, 78, 79, and 85 being the most immunogenic.

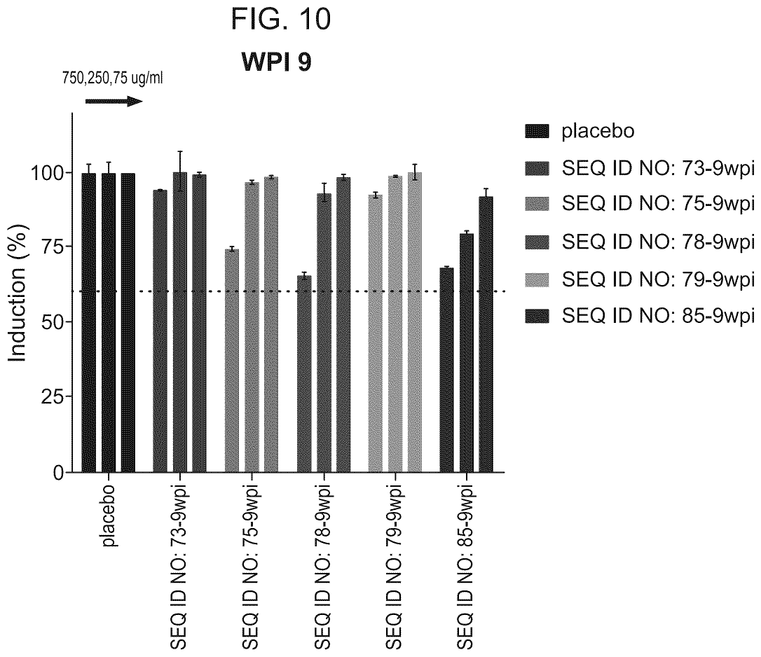

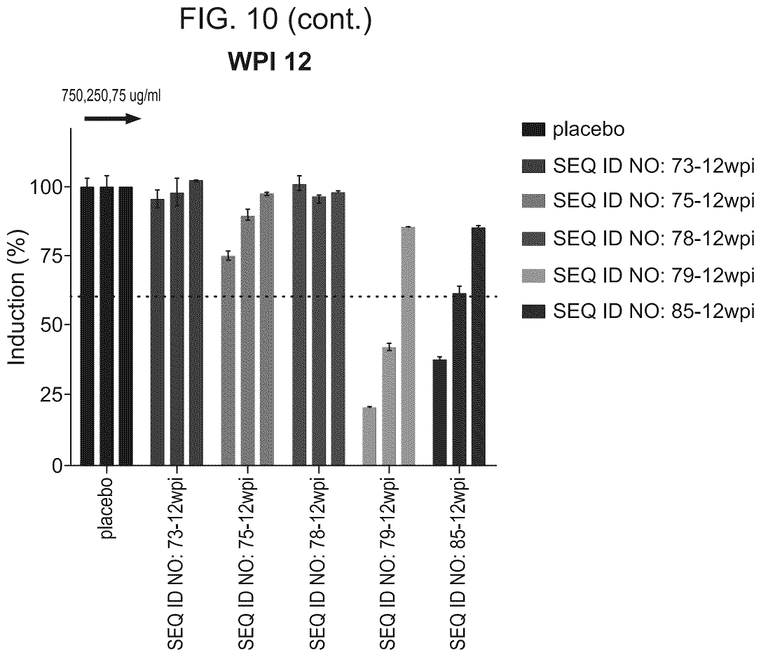

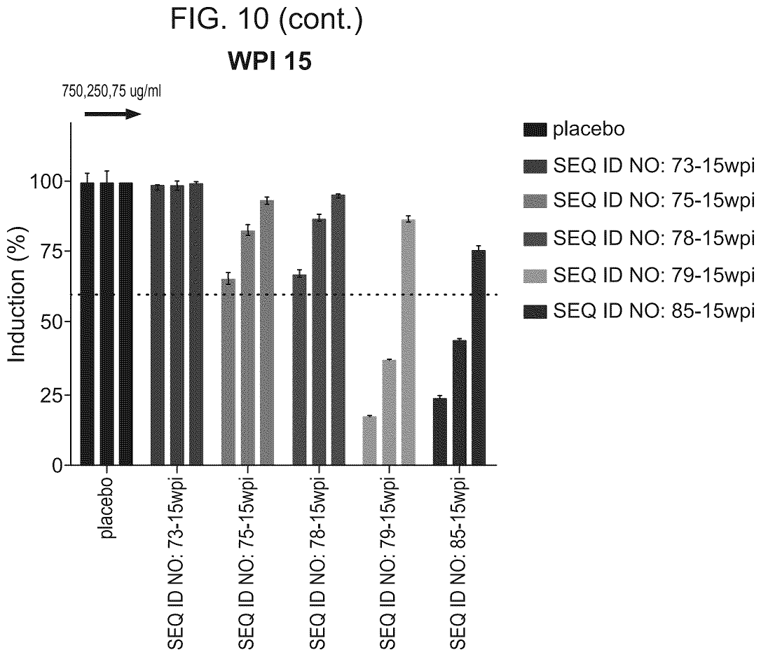

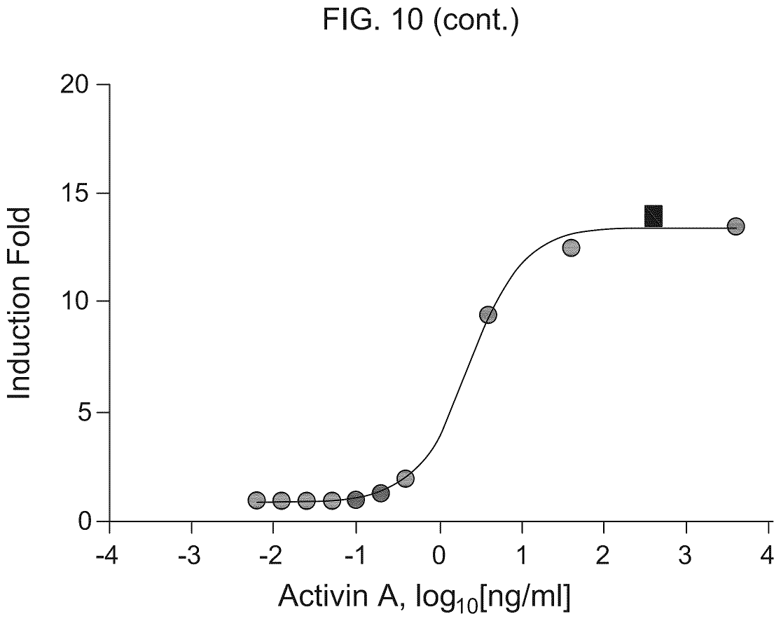

Antibodies were generated by immunization of Guinea pigs with the activin A peptide immunogen constructs listed in FIG. 10. IgGs obtained from the Guinea pigs and purified at 9-, 12-, and 15-weeks post-immunization (wpi) were tested for their ability to inhibit activin A-induced SMAD2/3 signaling in a cell-based reporter system at 750, 250, and 75 μg/mL. The results show time and concentration-dependent inhibition of activin A-induced signaling for most constructs, in particular the following constructs: SEQ ID NOs: 75, 78, 79, and 85.

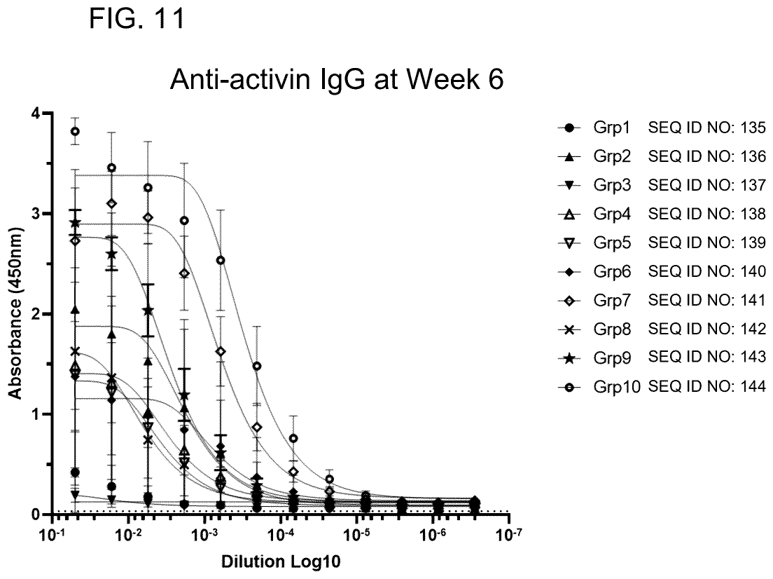

Further, the activin A peptide immunogen constructs listed in FIG. 11 (SEQ ID NO: 135-144) were injected IM into rats at weeks 0 and 3. Each injection consisted of 300 μg of peptide immunogen construct formulated in Adju-Phos with 100 μg of CpG1. Serum samples obtained at 6 weeks-post immunization (wpi) were diluted as indicated in the figure and analyzed on plates coated with full-length activin A protein. OD 450 readings of sera from individual animals were averaged together. FIG. 11 shows that the immunizations resulted in the production of antibodies that recognize activin A, with SEQ ID NOs: 141, 143, and 144 showing the highest activity.

### Example 3

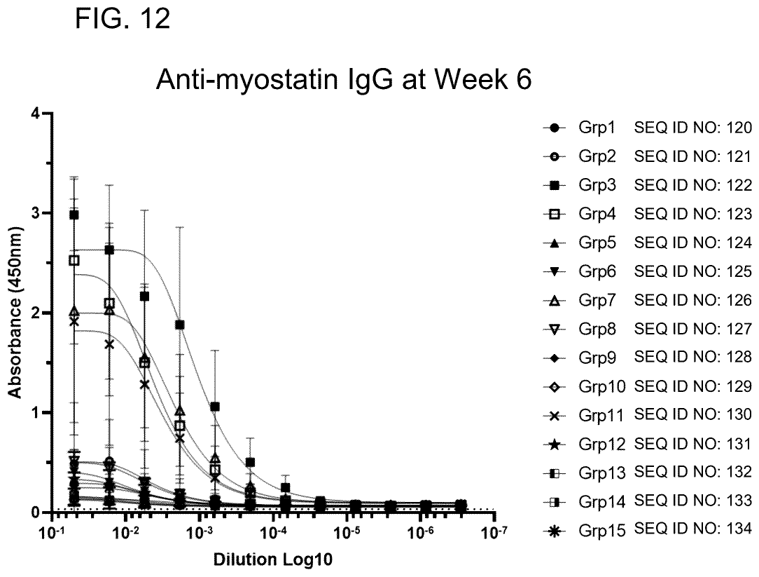

The myostatin peptide immunogen constructs listed in FIG. 12 (SEQ ID Nos: 120-134) were injected IM into rats at weeks 0 and 3. Each injection consisted of 300 μg of peptide immunogen construct formulated in Adju-Phos with 100 μg of CpG1. Serum samples obtained at 6 weeks-post immunization (wpi) were diluted as indicated in the figure and analyzed on plates coated with full-length myostatin protein. OD 450 readings of sera from individual animals were averaged together. FIG. 12 shows that the immunizations resulted in the production of antibodies that recognize myostatin, with SEQ ID NOs: 122, 123, 126, and 130 showing the highest activity.

### Example 4

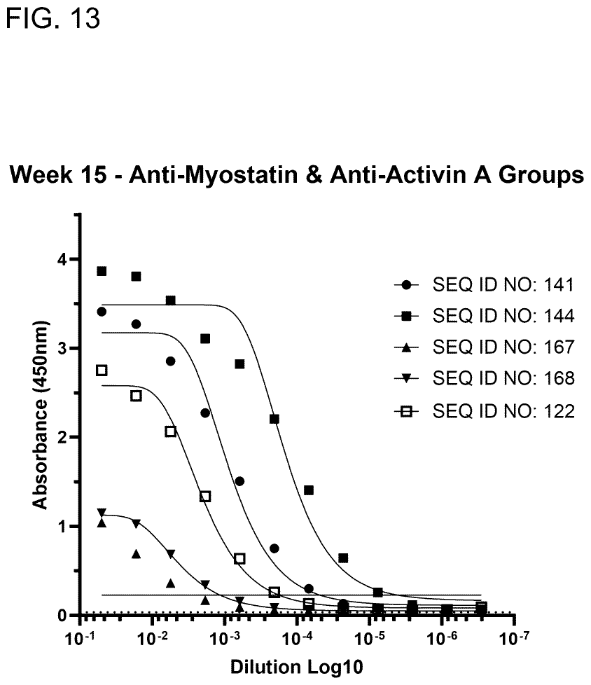

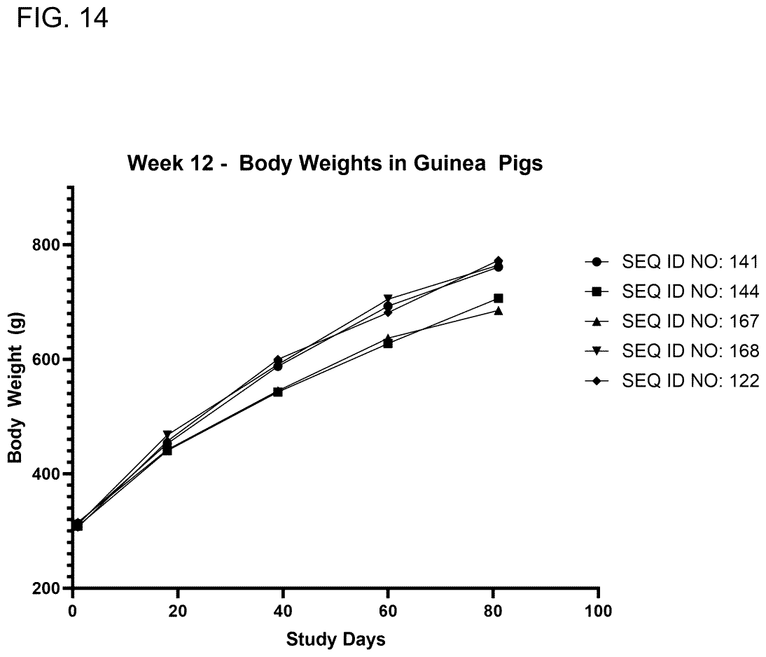

Guinea pigs were individually injected IM with a myostatin construct (SEQ ID NOs: 122, 167, or 168) or with an activin A construct (SEQ ID NOs: 141 or 144) at weeks 0, 3, 6, 9, and 12. Each injection consisted of 300 μg of peptide immunogen construct formulated in Adju-Phos with 100 μg of CpG1. Serum samples obtained at 15 weeks-post immunization (wpi) were diluted as indicated in the figure and analyzed on plates coated with full-length myostatin or activin A protein. OD 450 readings of sera from individual animals were averaged together. The immunogenicity results are shown in FIG. 13. The body weights of the guinea pigs in this study were also tracked over time and FIG. 14 shows that guinea pigs immunized with constructs of SEQ ID NOs: 141, 122, and 168 gained more weight than those immunized with the other constructs shown.

### Example 5

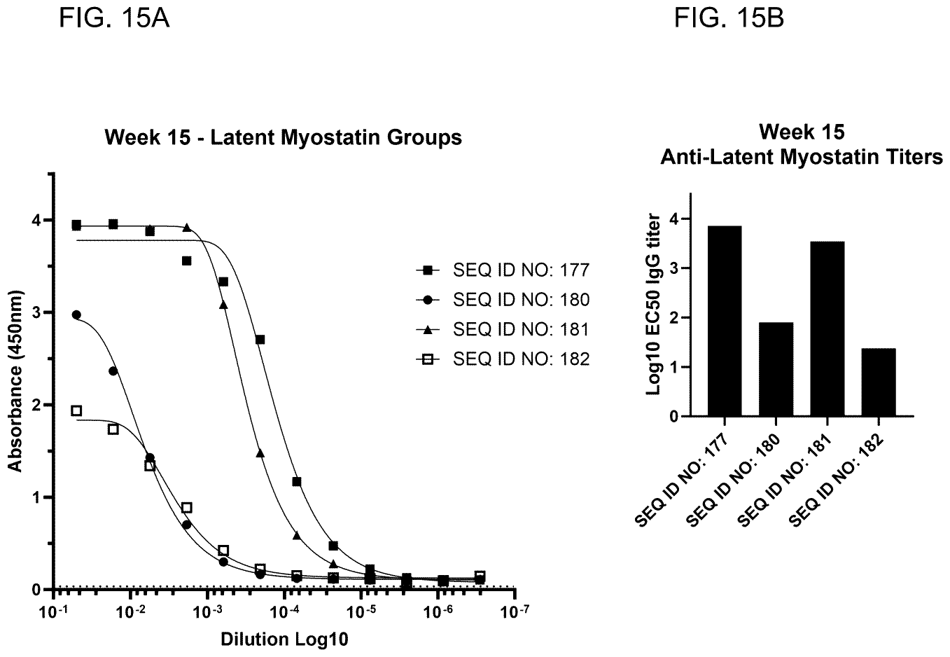

Additional myostatin constructs were generated with the goal of reducing or removing cross-reactivity with GDF-11. Specifically, constructs were made to target latent myostatin, promyostatin, and mature myostatin. Additional constructs were also made to target activin A. In FIGS. 15, 16, 17, and 18, the immunogenicity and antibody titers were determined as follows. The myostatin or activin A peptide immunogen constructs shown in each figure were injected IM into individual rats at weeks 0, 3, 6, and 12. Each injection consisted of 300 μg of peptide immunogen construct formulated in Adju-Phos with 100 μg of CpG1. For immunogenicity assays, serum samples obtained at 15 weeks-post immunization (wpi) were diluted as indicated in the figure and analyzed on plates coated with either latent myostatin, promyostatin, mature myostatin, or mature activin A. OD 450 readings of sera from individual animals were averaged together. To determine antibody titers, pre-immune sera and sera from immunized rats were collected at week 15 according to experimental vaccination protocols and stored at −80° C. 96-well microtiter plates were coated with 1 μg/mL of protein (mature myostatin, latent myostatin, promyostatin, mature activin A, or GDF-11) diluted in PBS. Coated plates were sealed and incubated at 4° C. overnight. Plates were removed from incubation, washed four times with PBS containing 0.05% by volume TWEEN® 20 (PBS-T) and then blocked by adding 250 μL of SuperBlock™ blocking buffer followed by a 2-hour incubation, shaking (500 rpm) at 37° C. Blocking buffer was removed after incubation and plates were washed once with PBS-T before the addition of sera. Pre-immune and immune serum samples were thawed at 4° C. and then diluted 1:20 in ELISA diluent (2% BSA by weight in PBS). Diluted sera were titrated by 8- or 12-point serial 3-fold dilution in a dilution block and 100 μL aliquots of each serum dilution were added to the 96 well plate in duplicate. Diluted sera were incubated for 1 hour at 25° C. and then washed six times with PBS-T. Horseradish peroxidase (HRP)-conjugated species-specific goat anti-IgG was used as a labeled tracer to bind with the antibody/peptide antigen complex formed in positive wells. One hundred microliters of the HRP linked goat anti-species IgG at a pre-titered optimal dilution in ELISA diluent was added to each well and incubated at 25° C., shaking (500 rpm) for 45 minutes. The wells were then washed six times with PBS-T to remove unbound antibody and reacted with 100 μL of the substrate (1-Step™ TMB ELISA Substrate) for 15 minutes. This substrate mixture was used to detect the peroxidase label by forming a colored product. Reactions were stopped by the addition of 100 μL of 1M H₂SO₄ and absorbance at 450 nm (A450) determined. For the determination of antibody titers of the vaccinated animals that received the various myostatin or activin A derived peptide immunogens, 3-fold serial dilutions of sera from 1:20 to 1:3542940 were tested, and the titer of a tested serum was determined as the EC50 from a nonlinear four-parameter curve and expressed as the Log 10 EC50 titer.

FIG. 15 shows the immunogenicity and antibody titers resulting from immunizations with the latent myostatin constructs of SEQ ID Nos: 177, 180, 181, or 182. FIG. 15A shows that, at week 15, immunization with all of the constructs resulted in production of antibodies against latent myostatin, but the immunizations with SEQ ID NO: 177 and SEQ ID NO: 181 resulted in the strongest production of antibodies that recognize latent myostatin. FIG. 15B shows that immunization with all four constructs led to high antibody titers, but immunizations with SEQ ID NO: 177 and SEQ ID NO: 181 resulted in the highest antibody titers.

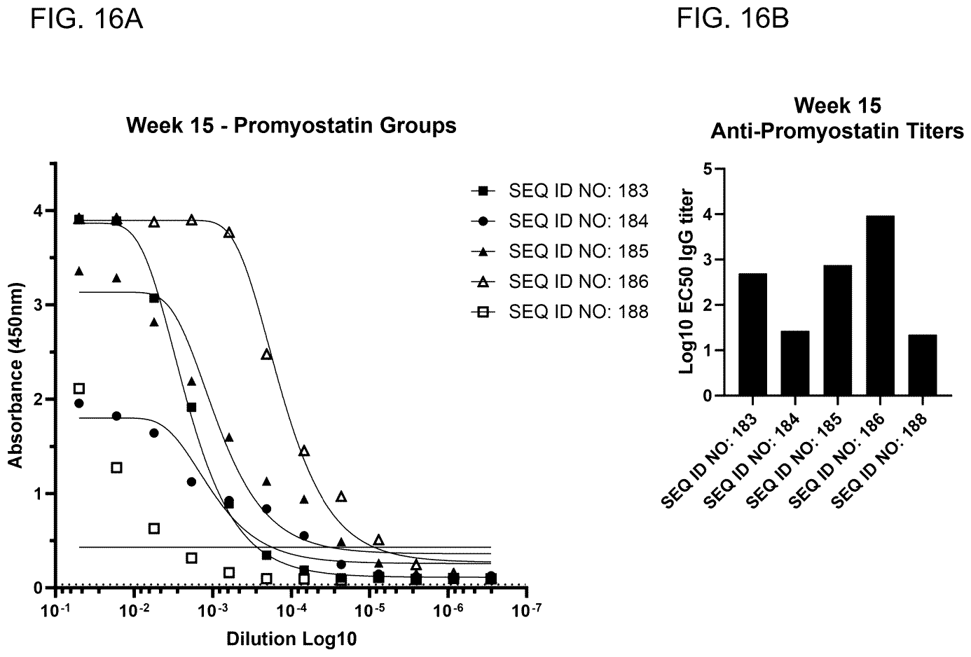

The immunogenicity and antibody titers resulting from immunizations with the promyostatin peptide immunogen constructs of SEQ ID NOs: 183, 184, 185, 186, or 188 are shown in FIG. 16. FIG. 16A and FIG. 16B show that the immunizations all constructs resulted in production of antibodies against promyostatin, but immunizations with SEQ ID NO: 183, SEQ ID NO: 185, and SEQ ID NO: 186 resulted in the strongest production of antibodies that recognize promyostatin and the highest antibody titers at week 15.

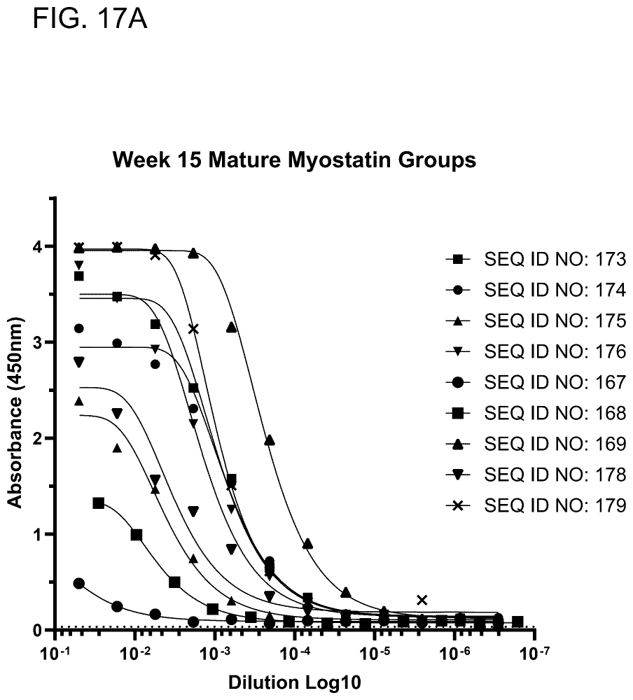

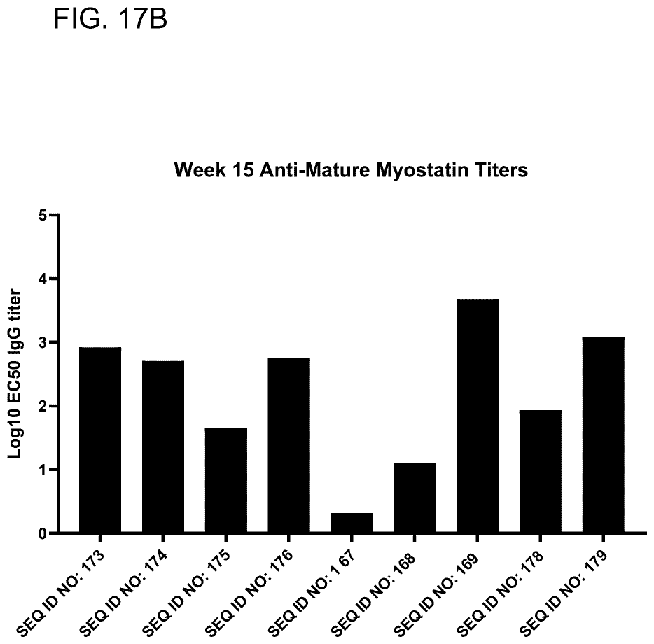

The constructs targeting mature myostatin of SEQ ID NO: 167, 168, 169, 173, 174, 175, 176, 178, or 179 were injected into individual rats at weeks 0, 3, 6, and 9 and immunogenicity and antibody titers against mature myostatin were determined at week 15. FIG. 17A shows that the majority of the constructs resulted in production of antibodies that recognize mature myostatin, but immunizations with SEQ ID NO: 169, SEQ ID NO: 173, SEQ ID NO: 174, SEQ ID NO: 176, and SEQ ID NO: 179 resulted in the strongest production of antibodies. As shown in FIG. 17B, antibody titers were the highest in rats immunized with SEQ ID NO: 169, SEQ ID NO: 173, SEQ ID NO: 174, SEQ ID NO: 175, SEQ ID NO: 176, SEQ ID NO: 178, and SEQ ID NO: 179.

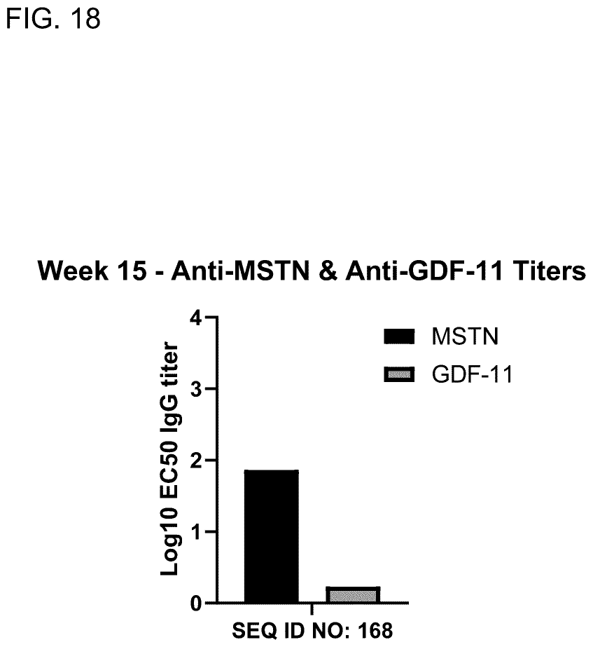

SEQ ID NO: 168 was injected IM into rats at weeks 0, 3, 6, 9, and 12. At week 15, antibody titers against mature myostatin and mature GDF-11 were determined, and the results are shown in FIG. 18. The rats immunized with SEQ ID NO: 168 had high titers of antibodies that recognize mature myostatin but low titers of antibodies that recognize GDF-11.

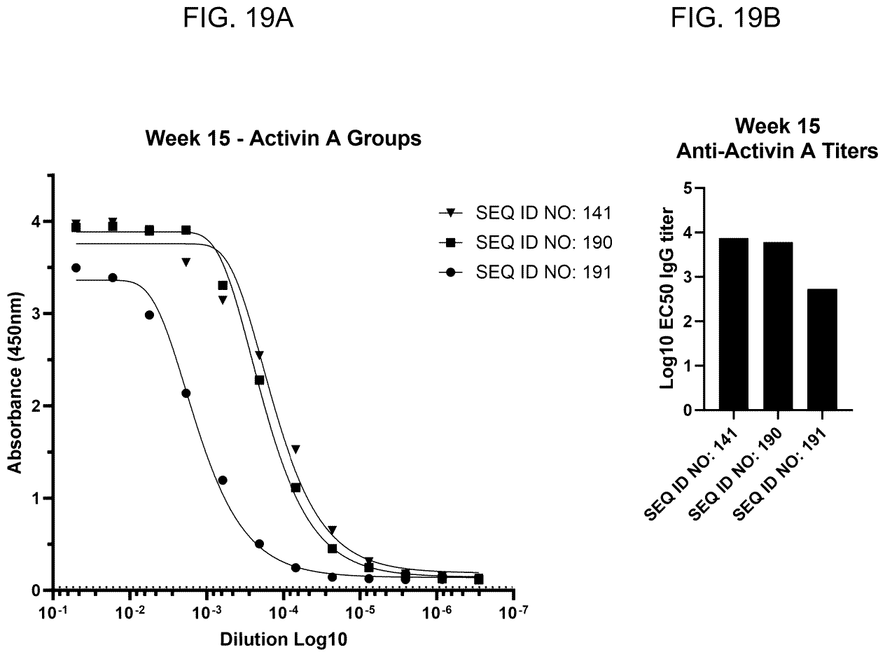

Lastly, rats were immunized at weeks 0, 3, 6, 9, and 12 with the activin A constructs of SEQ ID NOs: 141, 190, or 191. Immunization with all three constructs resulted in the production of antibodies (FIG. 19A) and high antibody titers (FIG. 19B) at week 15, with SEQ ID NO: 141 and SEQ ID NO: 190 showing the greatest effect.

### Example 6

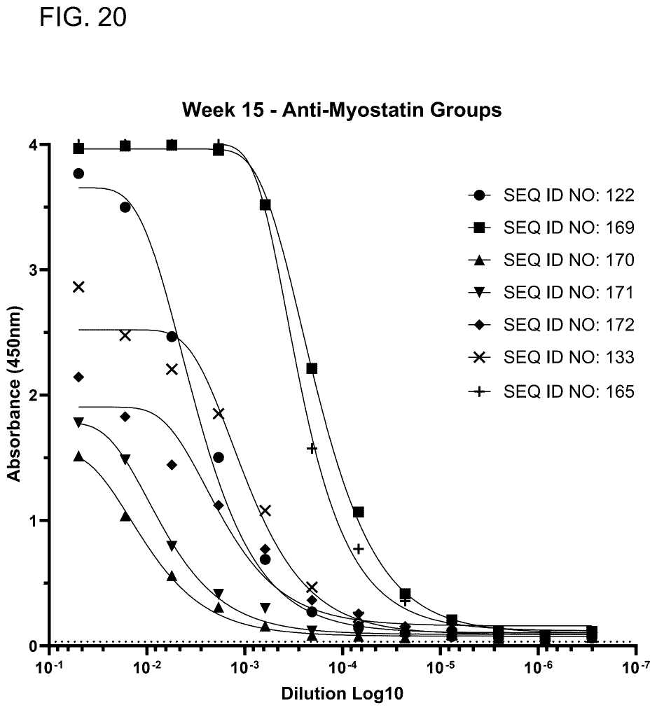

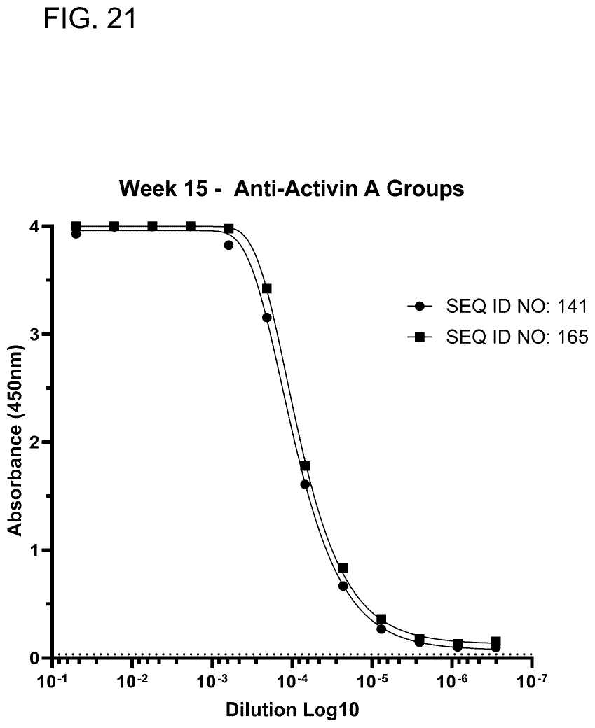

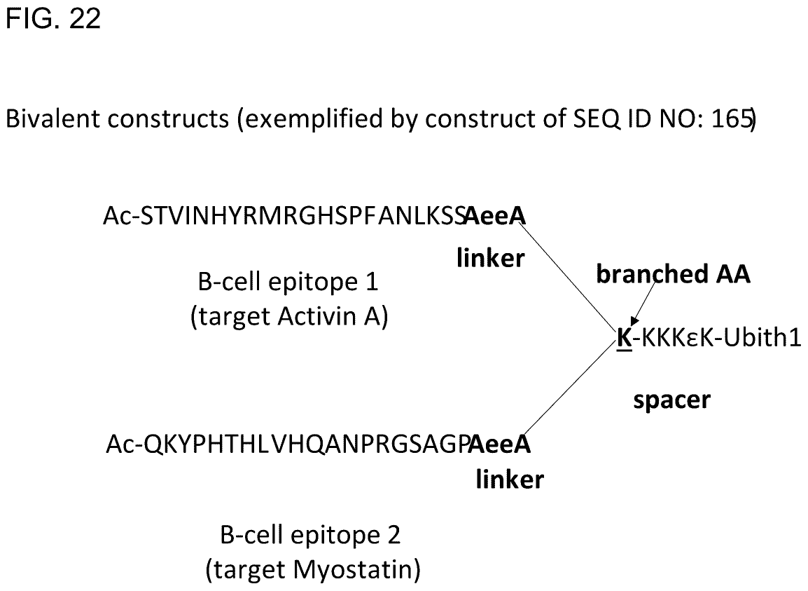

A combo myostatin—activin A peptide immunogen construct was generated using the myostatin B cell epitope of SEQ ID NO: 97 and the activin A B cell epitope of SEQ ID NO: 116. The resulting construct has the following amino acid sequence: Ac-STVINHYRMRGHSPFANLKSSAeeA(Ac-QKYPHTHLVHQANPRGSAGPAeeA)K-KKKrKUbith1 (SEQ ID NO: 165), where the peptide between ( ) indicates that it is off the side chain of the Lys and not the alpha amino group, AeeA is a peg spacer, and the Ac indicates that the construct is acetylated on its terminal NH2. SEQ ID NO: 165 was injected IM into rats at weeks 0, 3, 6, 9, and 12. Each injection consisted of 300 μg of peptide immunogen construct formulated in Adju-Phos with 100 μg of CpG1. Serum samples obtained at 15 weeks-post immunization (wpi) were diluted as indicated in the figure and analyzed on plates coated with full-length myostatin protein (FIG. 20) or full-length activin A protein (FIG. 21). OD 450 readings of sera from individual animals were averaged together. In the same experiment, additional rats were individually injected with other myostatin constructs (SEQ ID NOs: 122, 133, 169, 170, 171, or 172) or other activin constructs (SEQ ID NO: 141) at the same dose and timing and the immunogenicity of those constructs was also evaluated. FIGS. 20 and 21 show that the immunizations with SEQ ID NO: 165 resulted in the production of antibodies that recognize both myostatin and activin A. Immunization with SEQ ID NO: 165 generates a higher antibody titer against myostatin than immunization with SEQ ID NO: 122 (construct containing SEQ ID NO: 97) and a similar antibody titer against activin A compared to immunization with SEQ ID NO: 141 (construct containing SEQ ID NO: 116). SEQ ID NO: 169 also generates a high antibody titer at 15 weeks.

### Example 7

Sarcopenia is characterized by the progressive loss of skeletal muscle mass, strength, and physical performance that occurs with aging. The present example demonstrates the effect of myostatin (“MSTN”) and/or activin A (“Act A”) inhibition, using peptide immunogen constructs of the present invention, on muscle mass, strength, and performance in growth hormone (“GH”) sufficient and GH deficient subjects. The present example examined how GH presence impacts MSTN and Act A inhibition, and demonstrated that peptide immunogen constructs of the present invention increase muscle strength/performance in both GH sufficient and GH deficient subjects. The present example also demonstrated that peptide immunogen constructs of the present invention increase muscle mass in GH sufficient subjects (but not GH deficient subjects).

### Experimental Animals and Experimental Design

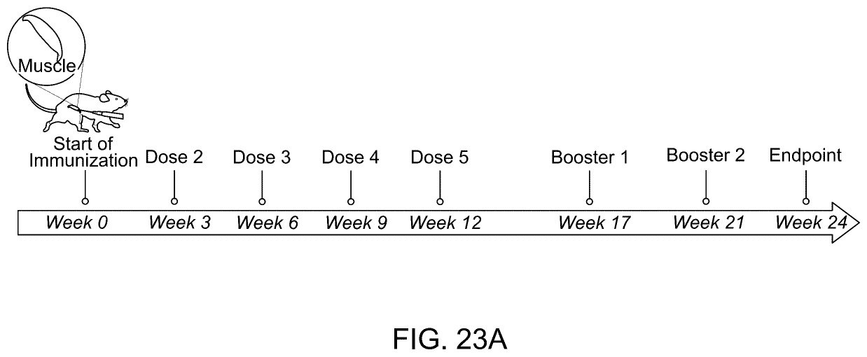

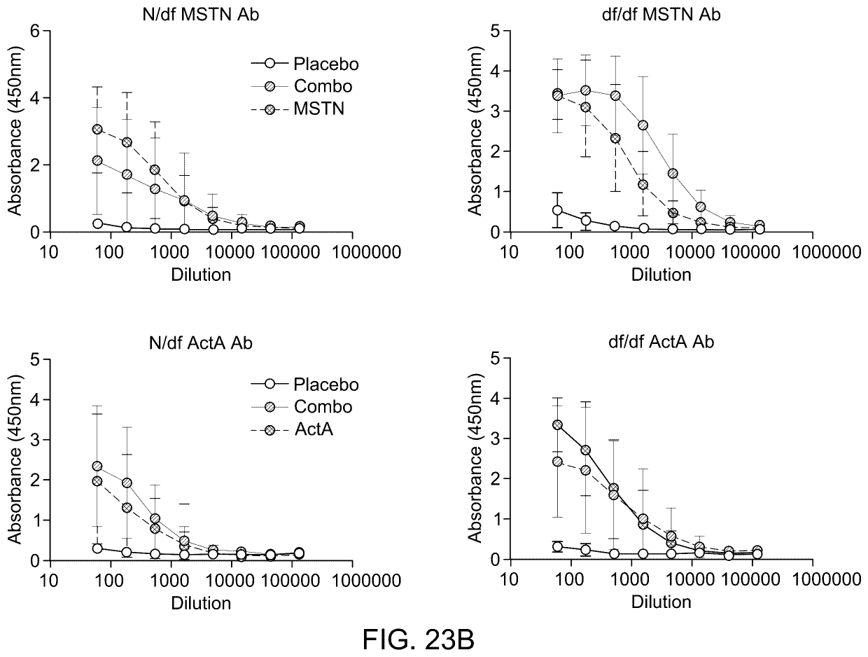

GH-deficient Ames dwarf (df/df) mice and normal littermate counterparts (control) (n=5-8; 8-14 months old; females) were housed in a pathogen-free, temperature and light-controlled environment with access to food (Rodent Laboratory Chow 5001) and water. The mice were immunized every 3 weeks with a placebo or one of peptide immunogen constructs of SEQ ID NOs: 122 (“anti-MSTN”), 141 (“anti-Act A”), and 165 (“anti-MSTN and anti-Act A combination” or “combination”) for 12 weeks (i.e., at weeks 0, 3, 6, 9, and 12) and for 2 additional doses at weeks 17 and 21. Sera were collected every 3 weeks after each of the initial five doses and analyzed by ELISA for the presence of anti-MSTN or anti-Act A antibodies according to a schedule described in FIG. 23A. Functional tests were conducted. At 24 weeks after the first treatment timepoint, mice were anesthetized under 2.5% isoflurane to collect blood via cardiac puncture and were subsequently euthanized by cervical dislocation. Tissues were collected and snap-frozen in liquid nitrogen to be stored at −80° C. for further molecular processing.

### ELISA

For ELISA, recombinant human mature myostatin (Peprotech) or activin A (ACRO Biosystems) were used to coat high-bind 96 well plates (Maxisorp, Thermo Scientific) at a coating concentration of 1 g/mL in coating buffer (1×PBS, Thermo Scientific). Coated plates were allowed to incubate at 25° C. for 1 hour followed by overnight incubation at 4° C. Following incubation, plates were blocked by the addition of blocking buffer (Blocker™ BSA, Thermo Scientific) and incubated for 2 hours at 37° C. with shaking (500 rpm) on an orbital shaker. Following incubation, murine serum samples were diluted 1:20 in diluent (blocking buffer diluted 1:5 in 1×PBS) and then used to prepare 8- or 12-point, 3-fold serial dilutions. Diluted serum samples along with positive and negative control serum samples (diluted 1:20) were then added to the plate and incubated for 1 hour at 25° C. Following incubation, each well received detection antibody (sheep anti-mouse IgG, R&D Systems) diluted 1:4000 in diluent. Secondary antibody (donkey anti-sheep IgG HRP, R&D Systems) was diluted 1:10,000 then applied to each plate and incubated for 45 min at 25° C. After incubation, 3,3′,5,5′-tetramethylbenzidine (TMB) Ultra ELISA substrate (Thermo Scientific) was applied to each well, and the reaction was stopped after 15 min by addition of 2 N H₂SO₄. Absorbance was measured at 450 nm on a SpectraMax iD5 plate reader (Molecular Devices) within 10 min of halting the reaction. A nonlinear four-parameter curve fit was applied to each dilution series to calculate the antibody titer, expressed as the half-maximal effective concentration (EC50; SoftMax Pro, version 7.1 or GraphPad Prism, version 9.2). For titers below the lower detection limit, a titer of <20 (starting dilution factor) was reported and a value of 10 assigned to the sample to calculate the group mean. Assay plates were washed three times between all ELISA steps in 1×PBS-T (0.05%, Thermo Scientific) except for the final wash (following secondary antibody incubation) where the plates were washed six times in 1×PBS-T. Plates were covered during all incubation steps and all working volumes in the microplate were 100 μL/well, apart from the blocking buffer and wash buffer steps which used 200 μL/well.

### Grip Strength

Forelimb grip strength was measured using a force transducer (Bioseb, Model GT3). Mice were allowed to grip a wire grid connected to the transducer and were pulled away with a constant, steady motion until the grip was lost. Each mouse was given three trials and the average grip strength was calculated and normalized to the weight of the animal. The mice were rested between trials to reduce muscle fatigue.

### Rotarod

Mice were tested for endurance using a five-lane rotarod (Ugo Basile). The animals were trained for 3 days at a constant speed of 18 rpm for 120 seconds. Animals were then subjected to an accelerating rotarod test where the rotating drum accelerated from 5 rpm to 40 rpm for 300 seconds. The time to fall was recorded. When an animal rotated for three continuous rotations without running, it was scored as a fall.

### Body Composition Analysis

Body composition was assessed using a nuclear magnetic resonance (NMR) system LF50-BCA Minispec (Bruker Inc.). This body composition analyzer quantifies lean mass, fat mass and free body fluid. Mice were immobilized in a plastic tube and placed in the NMR scan chamber for a short period to complete the scan.

### RNA Extraction and RNAseq Analysis

Total RNA was isolated from the gastrocnemius muscle using TRIzol reagent (Invitrogen) according to the manufacturer's instructions. The sample quality and concentration were determined using the Epoch Gen5 plate reader. RNA from each group (n=4) of df/df GH-deficient and N/df control gastrocnemius was sent for sequencing. The data were processed using bioinformatics tools in usegalaxy.org and differential expression comparisons were conducted using EdgeR package.

### Metabolic Monitoring

Indirect calorimetry and physical activity data were recorded using the Promethion Cage Monitoring System (Sable Systems International). Measurements included oxygen consumption, carbon dioxide production, food and water intake, and infrared beam breaks as indicators of locomotion in individually housed animals, and the data were exported in 15-minute macro slices. CalR software was used to generate Energy expense plots from the raw data.

### Lipidomics Analysis

Gastrocnemius muscles from aged mice used for lipidomics analysis. Briefly, lipids were isolated and lipidomics data were obtained using multi-dimensional mass spectrometry-based shotgun lipidomics technology. The lipid content for each lipid was normalized to the total protein content of the sample.

### Statistical Analysis

The results are shown as mean±standard error of the mean (SEM). Statistical analysis was performed using GraphPad Prism 9.1.0 (221) software, employing one-way ANOVA with Dunnette's multiple comparisons test or an independent t-test for two-group comparisons. A P-value less than 0.05 was deemed statistically significant.

### Results

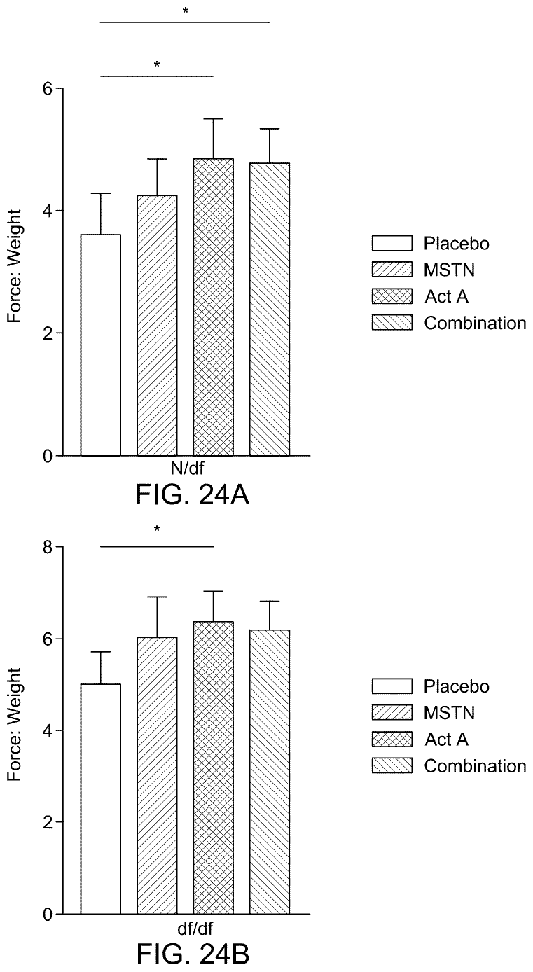

Antibody titer increases were observed after each dose. Highest levels of antibodies were present by week 15 (FIG. 23B). A grip strength assay was performed on the mice and the maximal forelimb force produced was recorded and normalized to the individual mouse's weight. Control animals with anti-MSTN and anti-Activin A antibodies showed increased levels of grip strength (P-MSTN=0.2213, P-ActA=0.0226, P-Combination=0.0345) (FIG. 24A). Increase in grip strength was not impacted by the absence of GH and lack of increase of lean mass, and was also observed in the df/df GH-deficient mice (P-MSTN=0.0750, P-ActA=0.0190, P-Combination=0.0573) (FIG. 24B).

To confirm the increased grip strength was due to the antibody levels present in these animals, a regression analysis was performed on individual mouse's antibody mean half maximal effective concentration (EC50) titer and their grip strength. The analysis revealed that observed grip strength showed a significant positive correlation to the levels of anti-MSTN and anti-Activin A antibodies (P-MSTN=0.004, P-ActA=0.01) (Experimental Table 1).

EXPERIMENTAL TABLE 1
Summary of Regression Analysis

| | Coefficient | SE | Z | P-value | 95% C.I. | |
|---|---|---|---|---|---|---|
| Log | 1.001 | 0.327 | 3.06 | 0.004 | 0.337 | 1.666 |
| (EC50), | | | | | | |
| MSTN | | | | | | |
| Constant | 2.397 | 0.86 | 2.79 | 0.009 | 0.647 | 4.147 |
| Log | 1.255 | 0.454 | 2.76 | 0.01 | 0.325 | 2.185 |
| (EC50), | | | | | | |
| ActA | | | | | | |
| Constant | 2.161 | 1.094 | 1.97 | 0.058 | -0.08 | 4.402 |

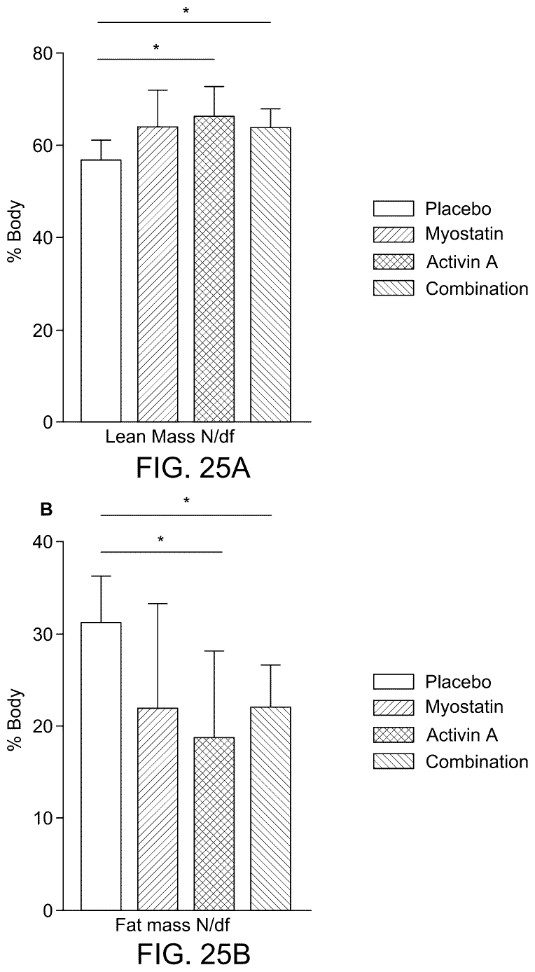

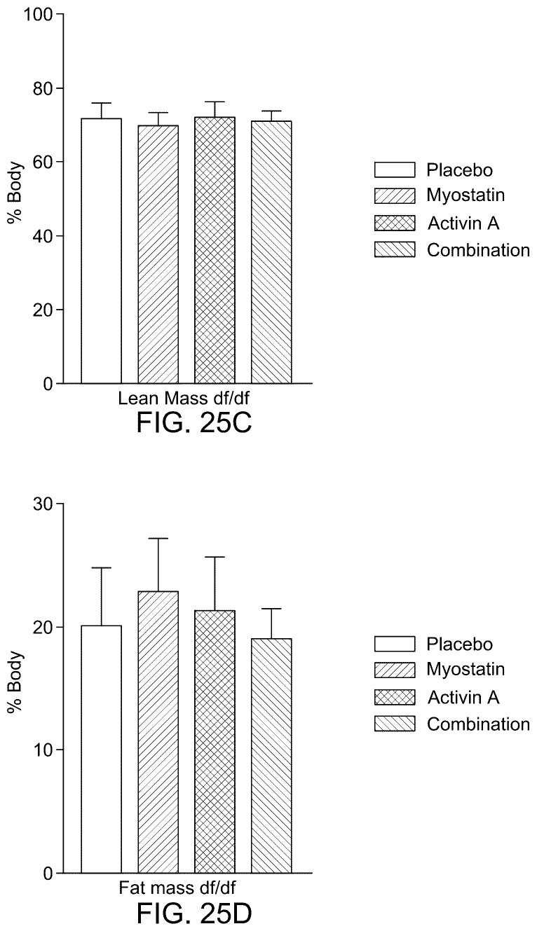

To determine whether increased grip strength was directly associated with immunization-induced gain of muscle mass, the animals were subjected to whole body composition analysis (BCA). BCA revealed that immunization led to lean mass gain (FIG. 25A) and fat mass loss (FIG. 25B) in mice with normal GH activity. However, in the absence of GH in the df/df mice, there was no change in overall lean mass (FIG. 25C) and fat mass (FIG. 25D) in response to immunization. Without wishing to be bound by any particular scientific theory, while GH-assisted lean mass gain appeared to have facilitated muscle strength increase in normal mice, in the absence of GH, myostatin and Activin A inhibition via immunization worked through a different mechanism to facilitate the observed increased muscle strength.

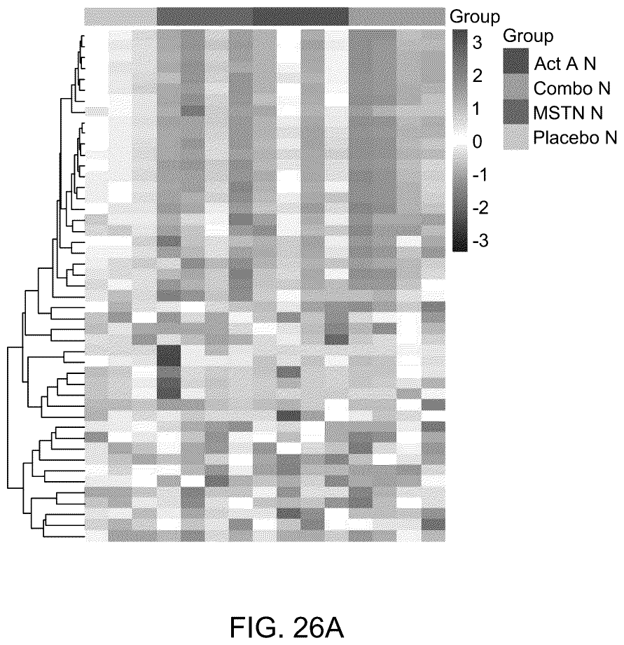

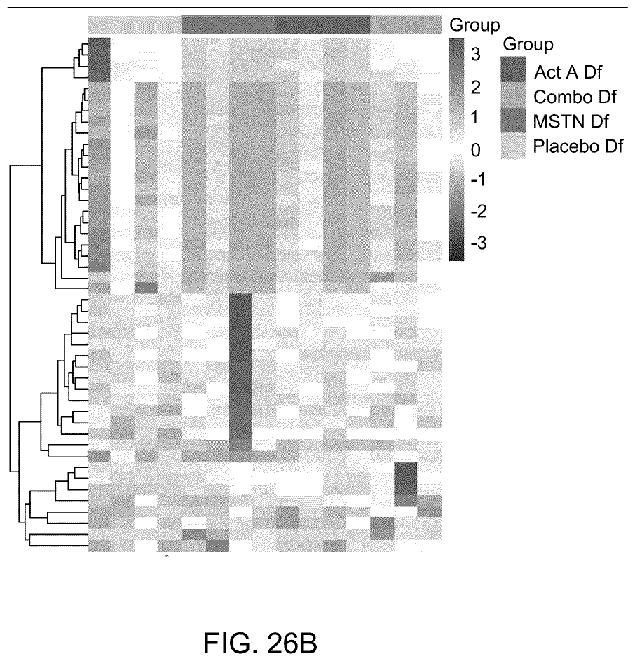

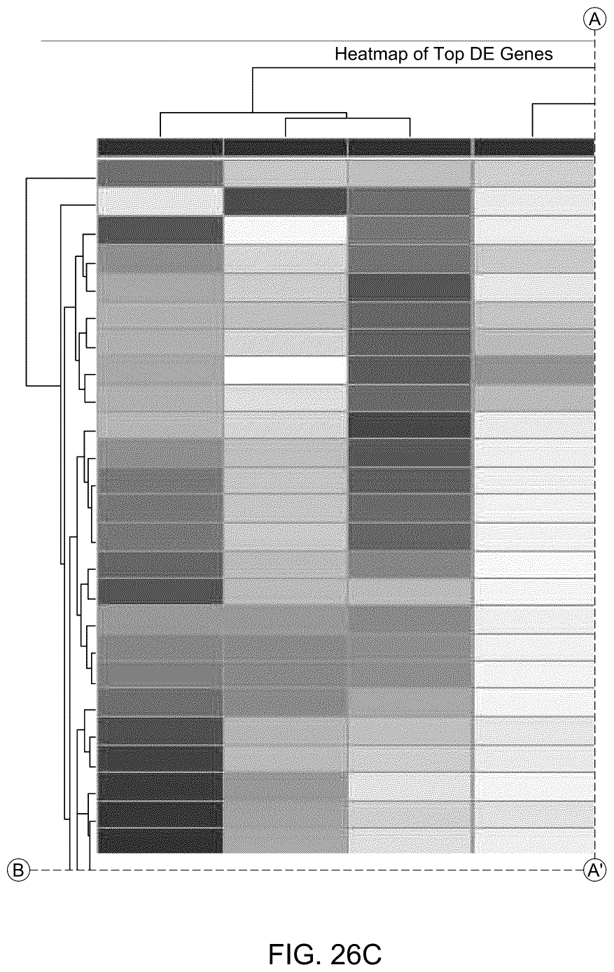

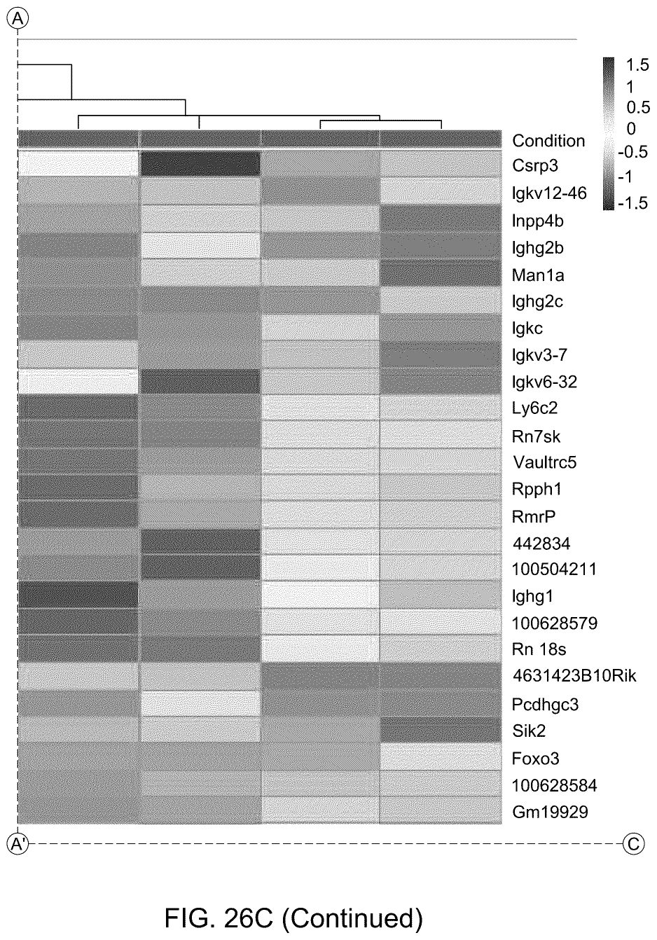

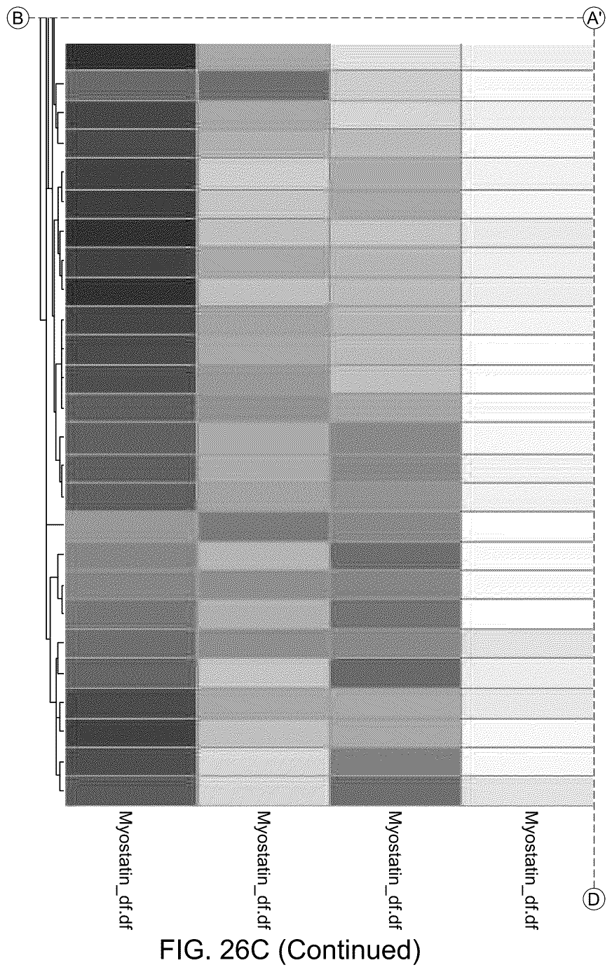

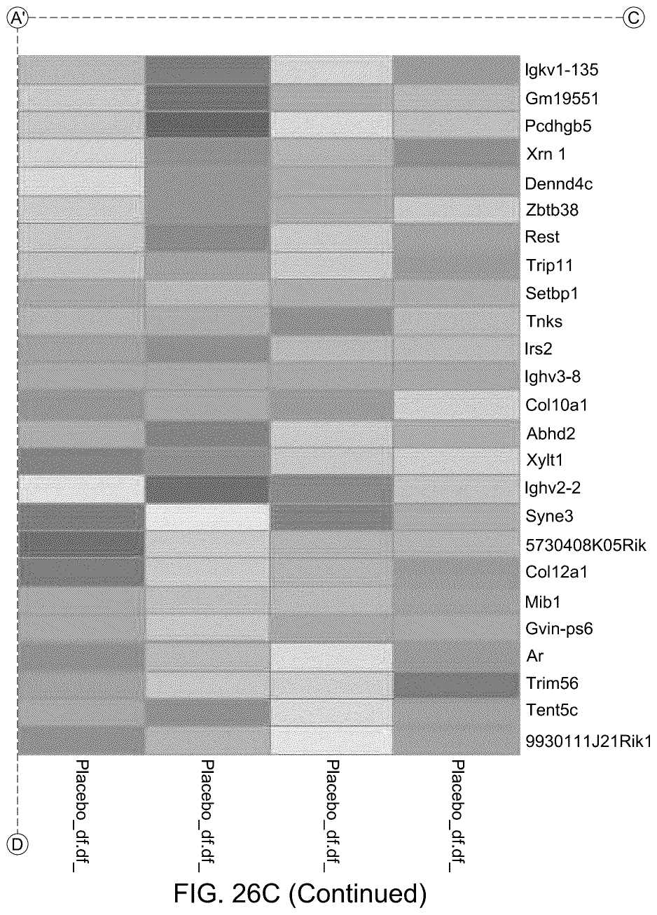

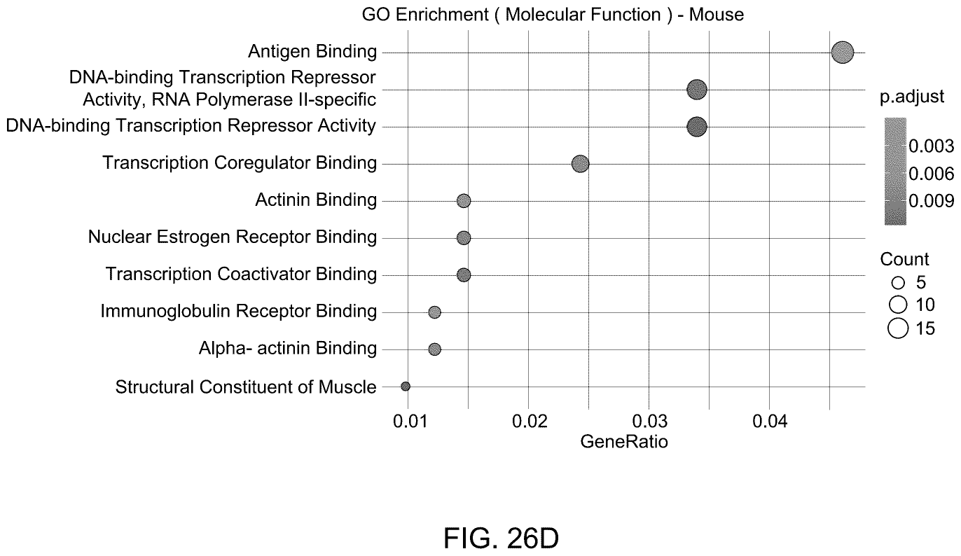

To gain insight into how long-term inhibition of MSTN and Activin A via immunization improves grip strength despite GH deficiency, RNA was isolated from the gastrocnemius muscle of the immunized animals and analyzed. There were no striking changes in the transcriptome in the normal control mice post-immunization (FIG. 26A). However, the gastrocnemius transcriptomic profile of the df/df mice changed in the immunized animals when compared with placebo controls (FIG. 26B). Gene expression changes detected were similar in the singular inhibition of MSTN (with peptide construct of SEQ ID NO: 122) or Activin A (with peptide construct of SEQ ID NO: 141), while the dual inhibition with combination therapy (with peptide construct of SEQ ID NO: 165) showed a different profile.

A comparison was made between placebo control and MSTN inhibition, as the largest number of differentially expressed genes was seen in this pairwise comparison. A gene oncology (GO) enrichment analysis for molecular function was performed (FIG. 26C). Among the hits for molecular functions, actinin binding and structural constituents of muscle were recognized as particularly relevant (FIG. 26D). Without being bound by theory, these findings point to structural remodeling in animals with the long-term inhibition of MSTN in the absence of GH that facilitated the muscle strength improvement in absence of muscle mass increase.

The data presented in this example showed that immunization with the peptide immunogen constructs of the present invention: (i) induced robust antibody production, (ii) increased muscle strength (such as grip strength) in immunized subjects both in the presence and absence of GH, (iii) increased lean muscle mass and decreased fat mass in subjects with normal GH action, but not in subjects with GH-deficiency, and (iv) induced post-immunization transcriptomic shift pointing to structural remodeling of the muscle in the absence of GH.

### Example 8

The present example demonstrates the effect of myostatin and/or activin A inhibition, using peptide immunogen constructs of the present invention, on muscle mass, strength, and performance in aged subjects. In this study, an immunization strategy similar to that described in Example 7 was applied to a mouse aging model characterized by natural, age-associated decline in the GH signaling axis and concomitant reduction in muscle mass and function. The present example demonstrated that peptide immunogen constructs of the present invention increased muscle strength in aged subjects. Similarly to GH-deficient df/df mice, aged subjects did not gain in lean mass or loss of fat mass in response to immunization.

### Experimental Animals and Experimental Design

Aged, 18-month-old C57BL/6 mice were subjected to immunotherapy, and studied until the mice reached the age of 24 months. Briefly, aged mice were immunized every 3 weeks with a placebo or one of peptide immunogen constructs of SEQ ID NOs: 122 (“anti-MSTN”), 141 (“anti-Act A”), and 165 (“anti-MSTN and anti-Act A combination,” or “combination”) for 12 weeks (i.e., at weeks 0, 3, 6, 9, and 12) and for 1 additional dose at week 17. Muscle strength, body composition, energy expense, and lipidomic analyses were performed as described in Example 7.

### Results

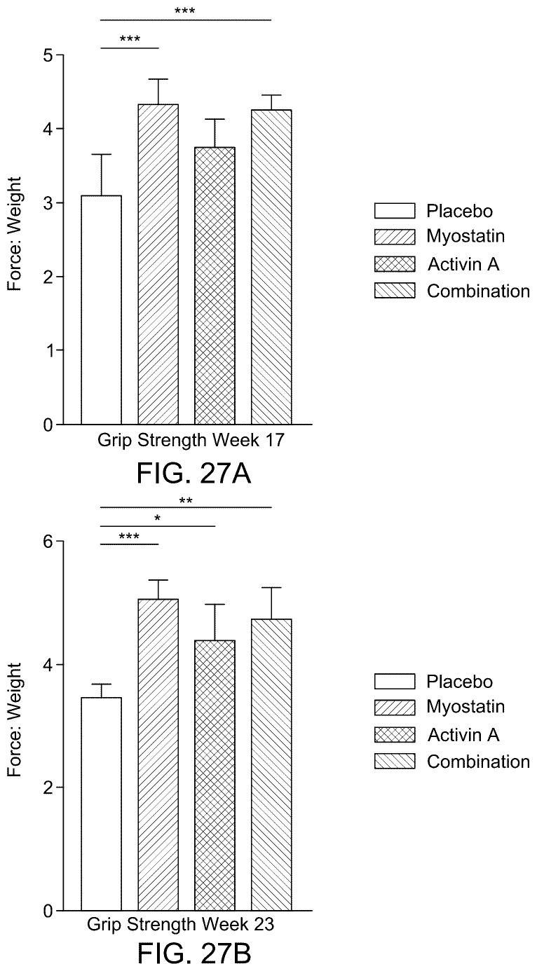

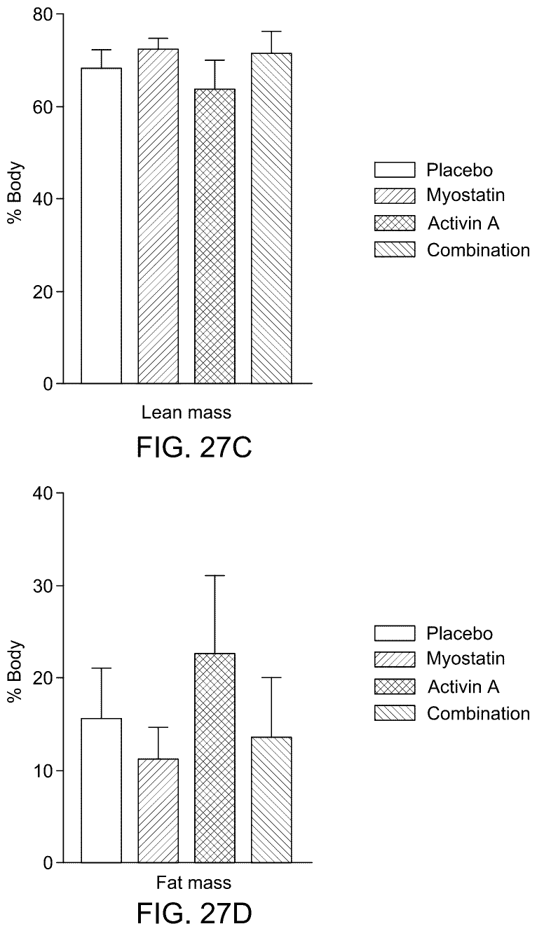

An increase in grip strength was observed in the aged animals as early as week 17 of treatment (FIG. 27A) and this increased strength was sustained through week 23 (FIG. 27B). No clear changes in lean mass or fat mass were observed in aged animals in response to immunization (FIGS. 27C-27D).

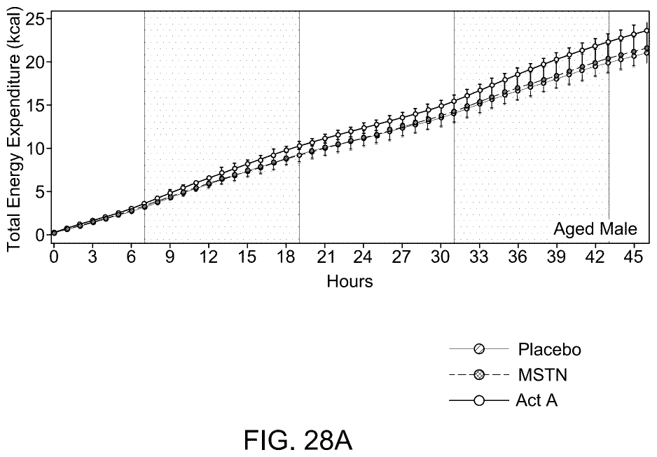

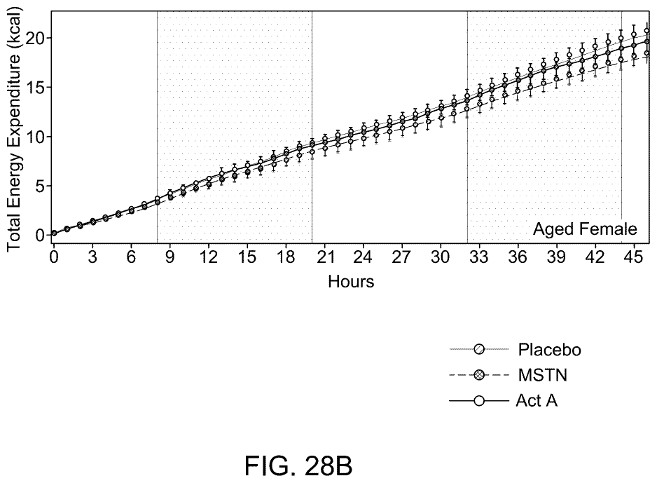

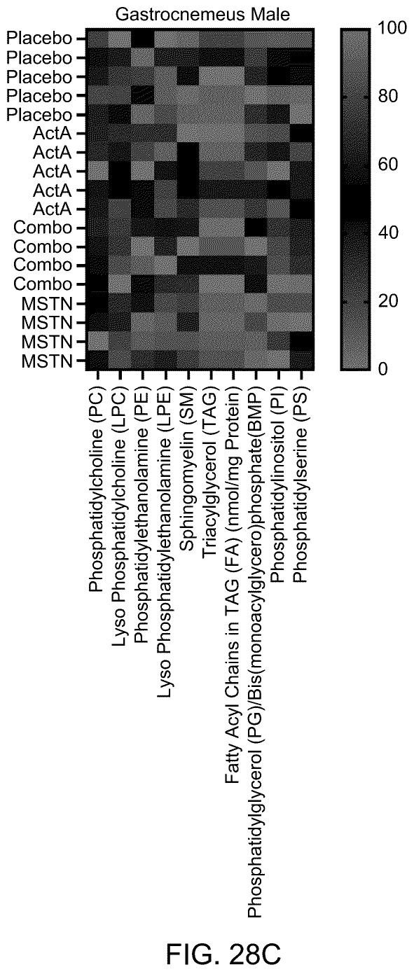

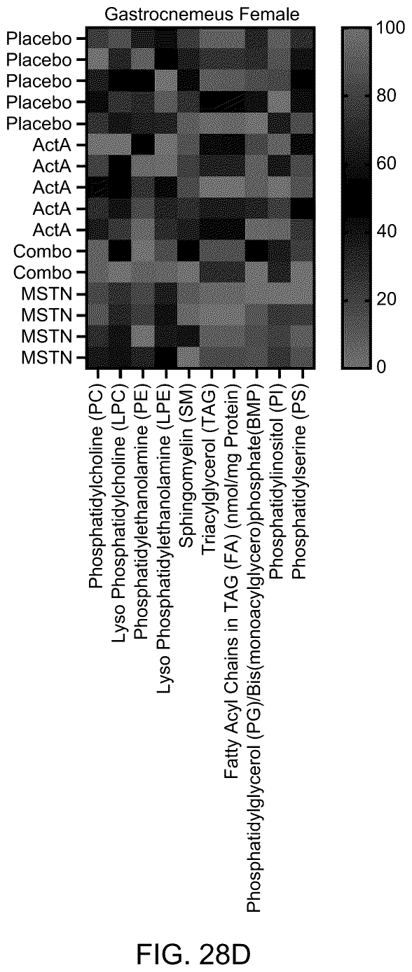

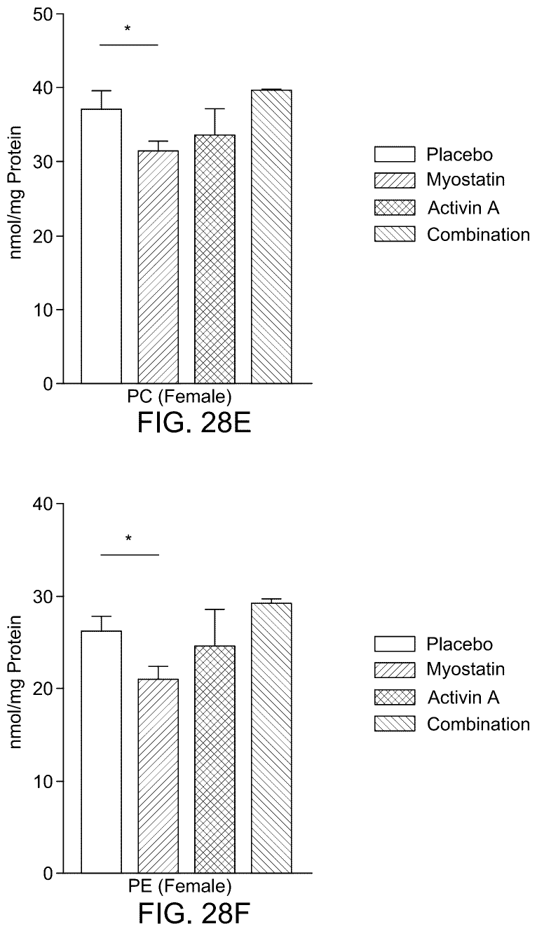

To understand potential changes in metabolism in the immunized animals, the animals were observed in a Promethion metabolic cage system. Single caged mice were observed for multiple consecutive days. The aged animals immunized against MSTN spent more energy over time in both male and female animals (FIGS. 28A-28B). With Act A inhibition, the energy expense increase was only observed in aged females but not in males (FIGS. 28A-28B).

Lipidomic analysis on the lipid extracted from the gastrocnemius muscle of the aged mice revealed a shift in some lipids with MSTN inhibition, including phosphatidylcholine (PC), lyso phosphatidylcholine (LPC), phosphatidylethanolamine (PE), lysophosphatidylethanolamine (LPE) and sphingomyelin (SM) (FIGS. 28C-28D). This shift was more noticeable in female aged mice than in the males. In particular, levels of PE and PC, which are highly relevant lipids in muscle function, were significantly reduced with MSTN inhibition in aged females (FIGS. 28E-28F).

The data presented in this example showed that immunization with the peptide immunogen constructs of the present invention: (i) increased muscle strength (such as grip strength) in aged subjects, (ii) resulted in an increased energy expense in aged subjects, and (iii) resulted in a lipidomic profile shift in the muscle.

Conclusions from Examples 7 and 8

Example 7 showed that in GH-deficient df/df mice, due to a mutation associated suppression of GH/IGF-1 axis, there was no increase in lean mass after immunization against MSTN and Act A. Yet, despite the lack of observed muscle mass growth, the immunized df/df mice showed improved grip strength, which is important for developing muscle performance therapies for older adults, who have significantly reduced GH levels (see Franco et al., 1989, New England Journal of Medicine 321(26):1797-1803; Anawalt et al., 2001, Endocrinol. Metab Clin North Am 30(3):647-669). These data showed that the peptide immunogen constructs described herein can successfully prevent or promote recovery from sarcopenia, which is highly relevant in geriatric practice when working with patients with suppression of the GH/IGF-1 axis due to aging. Although sarcopenia is often characterized by both loss of muscle mass and muscle strength in an accelerated manner, muscle strength decline is not proportional to muscle mass loss (see Menant et al., 2017, Osteoporosis International 28(1):59-70; Riviati, 2023, SAGE Open Med. 11, doi:10.1177/20503121231214650). Strength loss is very severe and likely contributes to increased fall risk and frailty (see Yeung et al., 2019, J Cachexia Sarcopenia Muscle 10(3):485-500). The data presented herein importantly indicate that it is possible to enhance muscle strength regardless of lean mass gains (possibly due to muscle tissue remodeling rather than mass increase). In addition to improved muscle strength, a higher percentage of the immunized df/df mice showed increased endurance in rotarod trials, compared to the placebo-treated mice.

Importantly, the immunization strategy provided herein was effective in aged murine models, where immunization initiated in 18-month-old mice yielded benefits by the time they reached 24 months of age. This enhanced muscle performance was persistent, with increased grip strength 17 weeks following the first treatment dose and by week 23 this increase was still observable. In the aged model, no notable increases in lean mass were observed, and thus this enhanced performance could have been attained in a similar way as in the GH-deficient df/df mice (without muscle mass increase).

The lipidomic analysis of the gastrocnemius muscles of the aged mice showed that, with inhibition of myostatin, aged female gastrocnemius muscles displayed significantly reduced PE and PC, two key lipids implicated in muscle performance. It has been shown previously that athletes in recovery after exercise have reduced levels of PE and PC, and this recovery period is when most of the muscle remodeling occurs (see Newsom et al, 2016, J Appl Physiol. 120:1355-1363).

In addition, no symptoms of autoimmune reaction were observed in the immunized mice, eliminating the possibility of an adverse T cell reaction to the peptide immunogen constructs tested.

In conclusion, the data presented herein show that the peptide immunogen constructs provided herein were capable of enhancing muscle performance regardless of GH-associated muscle development or age. This supports use of the provided constructs in therapies directed to improving muscle performance such as for sarcopenia, including in older subjects.

TABLE 1
Amino Acid sequences of myostatin and fragments (myostatin B cell epitopes)
thereof*

| SEQ | Epitope | Sequence |
|---|---|---|
| 1 | Myostatin | MQKLQLCVYIYLFMLIVAGPVDLNENSEQKENVEKEGLCNACTW |
| | preproprotein | RQNTKSSRIEAIKIQILSKLRLETAPNISKDVIRQLLPKAPPLRELIDQ |
| | | YDVQRDDSSDGSLEDDDYHATTETIITMPTESDFLMQVDGKPKCC |
| | | FFKFSSKIQYNKVVKAQLWIYLRPVETPTTVFVQILRLIKPMKDGTR |
| | | YTGIRSLKLDMNPGTGIWQSIDVKTVLQNWLKQPESNLGIEIKALD |
| | | ENGHDLAVTFPGPGEDGLNPFLEVKVTDTPKRSRRDFGLDCDEHS |
| | | TESRCCRYPLTVDFEAFGWDWIIAPKRYKANYCSGECEFVFLQKYP |
| | | HTHLVHQANPRGSAGPCCTPTKMSPINMLYFNGKEQIIYGKIPAMV |
| | | VDRCGCS |
| 2 | Mature | DFGLDCDEHSTESRCCRYPLTVDFEAFGWDWIIAPKRYKANYCSG |
| | myostatin | ECEFVFLQKYPHTHLVHQANPRGSAGPCCTPTKMSPINMLYFNGK |
| | | EQIIYGKIPAMVVDRCGCS |
| 3 | Myostatin | CDEHSTESRSC |
| | peptide - aa272- | |
| | 282, | |
| | C281→S281 | |
| 4 | Myostatin | CLTVDFEAFGWDWIIAPKRYKAC |
| | peptide - aa285- | |
| | 307, | |
| | P285→C285 | |
| | and | |
| | N307→C307 | |
| 5 | Myostatin | CKYPHTHLVHQANPRGSAGC |
| | peptide - aa319- | |
| | 338, | |
| | Q319→C319 | |
| | and p338→C338 | |
| 6 | Myostatin | CINMLYFNGKEQIIYGKIC |
| | peptide - aa347- | |
| | 365, | |
| | P347→C347 | |
| | and | |
| | P365→C365 | |
| 7 | Myostatin | CFVFLQKYPHTHLVHQANPRGSAGC |
| | peptide - aa314- | |
| | 338, | |
| | E314→C314 | |
| | and | |
| | P338→C338 | |
| 8 | Myostatin | CEFVFLQKYPHTHLVHQANPRGSAGC |
| | peptide - | |
| | aa313- | |
| | 338,P338→C338 | |
| 9 | Myostatin | CESEFVFLQKYPHTHLVHQANPRGSAGC |
| | peptide - aa311- | |
| | 338, | |
| | G311→C311, | |
| | P338→C338, | |
| | C313→S313 | |
| 10 | Myostatin | CSGESEFVFLQC |
| | peptide - aa309- | |
| | 320, | |
| | K320→C320, | |
| | C313→S313, | |
| 95 | Myostatin | EFVFLQKYPH |
| | peptide - aa314- | |
| | 323 | |
| 96 | Myostatin | ECEFVFCQKY |
| | peptide - aa312- | |
| | 321, | |
| | L318→C318 | |
| | S-S bridge (i.e., | |
| | disulfide | |
| | linkage) | |
| 97 | Myostatin | QKYPHTHLVHQANPRGSAGP |
| | peptide - aa319- | |
| | 338 | |
| 98 | Myostatin | CKYPHTHLVHQANPRGSAGC |
| | peptide - aa319- | |
| | 338, | |
| | Q319→C319, | |
| | P338→C338 | |
| | S-S bridge | |
| 99 | Myostatin | ECEFVFCQKYPHTHLVH |
| | peptide - aa314- | |
| | 328, | |
| | L318→C318 | |
| | S-S bridge | |
| 100 | Myostatin | EFVFLQKYPHTHLVHQANPRGSAGP |
| | peptide - aa314- | |
| | 338 | |
| 101 | Myostatin | CEFVFLQKYPHTHLVHQANPRGSAGPC |
| | peptide - aa313- | |
| | 339 | |
| | S-S bridge | |
| 102 | Myostatin | EFVFLQKYPHTHLVHQA |
| | peptide - aa314- | |
| | 330 | |
| 103 | Myostatin | Ahx-KYPHTHLVHQANPRGSAGE |
| | peptide - aa319- | |
| | 338, | |
| | Q319→Ahx319, | |
| | P338→E338 | |
| | Head to tail | |
| | linkage | |
| 104 | Myostatin | QKYPCTHLVHQANPRGSC |
| | peptide - aa319- | |
| | 336, | |
| | H323→C323, | |
| | A336→C336 | |
| | S-S bridge | |
| 105 | Myostatin | QKYPDTHLVHQANPRGSDap |
| | peptide - aa319- | |
| | 336, | |
| | H323→D323, | |
| | A336→Dap336 | |
| | Lactam linkage | |
| 106 | Myostatin | PHTHLVHQANPRGSAE |
| | peptide - aa322- | |
| | 337, | |
| | G337→E337 | |
| | Head to tail | |
| 107 | Myostatin | EFVFLQKY |
| | peptide - aa314- | |
| | 321 | |
| | Lactam side | |
| | chain to side | |
| | chain linkage | |
| 108 | Myostatin | EFVFLQKYPCTHLVHQANPRGSC |
| | peptide - aa314- | |
| | 336, | |
| | H323→C323, | |
| | A336→C336 | |
| | S-S bridge + | |
| | lactam | |
| 109 | Myostatin | DKYPHTHLVHQANPRGSAGDap |
| | peptide - aa319- | |
| | 338, | |
| | Q319→D319, | |
| | P338→Dap338 | |
| | Lactam | |
| 145 | Myostatin | YKANYSSGESEFEFLDab |
| | peptide - aa304- | |
| | 319, | |
| | C309→S309, | |
| | C313→S313, | |
| | V316→E316, | |
| | Q319→Dab319 | |
| | cyclized | |
| 146 | Myostatin | YKANYSSGESEFVFLQ |
| | peptide - aa304- | |
| | 319, | |
| | C309→S309, | |
| | C313→S313 | |
| 147 | Myostatin | QDabYPHTHLEHQANPR |
| | peptide - aa319- | |
| | 333, | |
| | K320→Dab320, | |
| | V327→E327 | |
| | cyclic | |
| 148 | Myostatin | QKYPHTHLVHQ |
| | peptide - aa319- | |
| | 329 | |
| 149 | Myostatin | HTHLVHQANPR |
| | peptide - aa323- | |
| | 333 | |
| 150 | Myostatin | QKYPHTHLCHQANPRC |
| | peptide - aa319- | |
| | 334, | |
| | V327→C327, | |
| | G334→ C334 | |
| | cyclic | |
| 151 | Myostatin | YKANYSSGESEFVFLQKYPHT |
| | peptide - aa304- | |
| | 324, | |
| | C309→S309, | |
| | C313→S313 | |
| 152 | Myostatin | YSSGESEFVFLQKYPHTHLV |
| | peptide - aa308- | |
| | 327, | |
| | C309→S309, | |
| | C313→S313 | |
| 153 | Myostatin | YKANYSSGESEFEFLDabKYPHT |
| | peptide - aa304- | |
| | 324, | |
| | C309→S309, | |
| | C313→S313, | |
| | V316→E316, | |
| | Q319→Dab319 | |
| 154 | Myostatin | YSSGESEFEFLDabKYEHTHLVOrn |
| | peptide - aa308- | |
| | 328, | |
| | C309→S309, | |
| | C313→S313, | |
| | V316→E316, | |
| | Q319→Dab319, | |
| | P322→E322, | |
| | H328→Orn328 | |
| 155 | Myostatin | LLPKAPPLRELID |
| | peptide - aa80- | |
| | 92 | |
| 156 | Myostatin | QDabYPHTHLE |
| | peptide - aa319- | |
| | 327 | |
| | K320→Dab320, | |
| | V327→E327 | |
| | cyclized | |
| 157 | Myostatin | QDabYPHTHLEH |
| | peptide - aa319- | |
| | 328, | |
| | K320→Dab320, | |
| | V327→E327 | |
| | cyclized | |
| 158 | Myostatin | LCELICQYDVQR |
| | peptide - aa87- | |
| | 98, | |
| | R88→C88, | |
| | D92→C92 | |
| 159 | Myostatin | VQRDDSSDGSLEDDDYH |
| | peptide - aa96- | |
| | 112 | |
| 160 | Myostatin | IITMPTESDFLM |
| | peptide - aa118- | |
| | 129 | |
| 161 | Myostatin | LNPFLEVKVTDT |
| | peptide - aa249- | |
| | 260 | |
| 162 | Myostatin | VTDTPKSRRDFGL |
| | peptide - aa257- | |
| | 270 | |
| 163 | Myostatin | FLEVKVTDTPK |
| | peptide - aa252- | |
| | 262 | |
| 164 | Myostatin | LAVTFPGPGEDGL |
| | peptide - aa237- | |
| | 249 | |
| 166 | Myostatin | TVFVQILRLIKPM |
| | peptide - aa168- | |
| | 180 | |

*Note:
“Ahx” is 6-aminohexanoic acid. “Dap” is L-2,3 diaminopropionic acid (CAS 1482-97-9). “Dab” is L-2,4 diaminobutyric acid (CAS 1883-09-6). “Orn” is L-Ornithine (CAS 3184-13-2).
*Each of the listed B cell epitope peptides is acetylated on the N-terminus and has an amide on the C-terminus (except for the peptides that are cyclized using the N-terminus amino group, such as peptides of SEQ ID NO: 103 (Ahx-KYPHTHLVHQANPRGSAGE) and SEQ ID NO: 106 (PHTHLVHQANPRGSAE)).

TABLE 2
Amino Acid sequences of activin A and fragments (activin A B cell epitopes)
thereof*

| SEQ | Epitope | Sequence |
|---|---|---|
| 11 | Activin A | MPLLWLRGFLLASCWIIVRSSPTPGSEGHSAAPDCPSCALAALP |
| | preproprotein | KDVPNSQPEMVEAVKKHILNMLHLKKRPDVTQPVPKAALLNA |
| | | IRKLHVGKVGENGYVEIEDDIGRRAEMNELMEQTSEIITFAESG |
| | | TARKTLHFEISKEGSDLSVVERAEVWLFLKVPKANRTRTKVTIRL |
| | | FQQQKHPQGSLDTGEEAEEVGLKGERSELLLSEKVVDARKSTW |
| | | HVFPVSSSIQRLLDQGKSSLDVRIACEQCQESGASLVLLGKKKKK |
| | | EEEGEGKKKGGGEGGAGADEEKEQSHRPFLMLQARQSEDHPH |
| | | RRRRRGLECDGKVNICCKKQFFVSFKDIGWNDWIIAPSGYHANY |
| | | CEGECPSHIAGTSGSSLSFHSTVINHYRMRGHSPFANLKSCCVPT |
| | | KLRPMSMLYYDDGQNIIKKDIQNMIVEECGCS |
| 12 | Mature activin A | GLECDGKVNICCKKQFFVSFKDIGWNDWIIAPSGYHANYCEGECP |
| | | SHIAGTSGSSLSFHSTVINHYRMRGHSPFANLKSCCVPTKLRPMS |
| | | MLYYDDGQNIIKKDIQNMIVEECGCS |
| 13 | Activin A peptide - | CDGKVNISC |
| | epitope design on | |
| | loop 1: | |
| | aa314-322, | |
| | C321→S321, | |
| | cyclized. (4-12) | |
| 14 | Activin A peptide - | CFKDIGWNDWIIAPSC |
| | epitope design on | |
| | loop 2: | |
| | aa329-344, | |
| | S329→C329, | |
| | G344→C344, | |
| | cyclized. (19-34) | |
| 15 | Activin A peptide - | CSLSFHSTVINHYRMRGHSPFANLKSC |
| | epitope design on | |
| | loop 3A: | |
| | aa364-390, | |
| | S364→C364, | |
| | cyclized. (54-80) | |
| 16 | Activin A peptide - | CPSHIAGTSGSSLSFHC |
| | epitope design on | |
| | loop 3B: | |
| | aa354-370, | |
| | S370 → C370, | |
| | cyclized. (44-60) | |
| 17 | Activin A peptide - | CTVINHYRMRGHSPFANLKSC |
| | epitope design on | |
| | loop 3C: | |
| | aa370-390, S370 → | |
| | C370, cyclized. | |
| | (64-80) | |
| 18 | Activin A peptide - | CSMLYYDDGQNIIKKDC |
| | epitope design on | |
| | loop 4: | |
| | aa399-415, | |
| | M399→C399, | |
| | I415→C415, | |
| | cyclized. (89-105) | |
| 110 | Activin A peptide - | DIGCNDWIIAC |
| | aa332-342, | |
| | W335→C335, | |
| | P342→C342, S-S | |
| | bridge | |
| 111 | Activin A peptide - | WNDWIIAPSG |
| | aa335-344 | |
| 112 | Activin A peptide - | STVINHYRMR |
| | aa370-379 | |
| 113 | Activin A peptide - | HYRMRGHSPF |
| | aa375-384 | |
| 114 | Activin A peptide - | HSPFANLKSS |
| | aa381-390, | |
| | C390→S390 | |
| 115 | Activin A peptide - | FKDIGWNDW |
| | aa330-338 | |
| 116 | Activin A peptide - | STVINHYRMRGHSPFANLKSS |
| | aa370-390, | |
| | C390→S390 | |
| 117 | Activin A peptide - | CTVINHYRMRGHSPFANLKSC |
| | aa370-390, | |
| | S370→C370, S-S | |
| | bridge | |
| 118 | Activin A peptide - | CFKDIGWNDWIIAPSC |
| | aa329-344, | |
| | S329→C329, | |
| | G344→C344, | |
| | S-S bridge | |
| 119 | Activin A peptide - | SFKDIGWNDWIIAPSG |
| | aa329-344 | |
| 187 | Activin A peptide - | YRMRGHSPFA |
| | aa376-385 | |
| 189 | Activin A peptide - | HYRMRGHSPFAN |
| | aa375-386 | |

*Each of the listed B cell epitope peptides is acetylated on the N-terminus and has an amide on the C-terminus (except for the peptides that are cyclized using the N-terminus amino group, such as peptides of SEQ ID NO: 103 (Ahx-KYPHTHLVHQANPRGSAGE) and SEQ ID NO: 106 (PHTHLVHQANPRGSAE)).

TABLE 3
Amino Acid Sequences of Pathogen Protein Derived Th Epitopes Including Idealized
Artificial Th Epitopes for Use in Myostatin and Activin A Peptide Immunogen Constructs

| Description | SEQ ID NO: | Sequence |
|---|---|---|
| Clostridium tetani1 Th | 19 | KKQYIKANSKFIGITEL |
| MvF1 Th | 20 | LSEIKGVIVHRLEGV |
| Bordetella pertussis Th | 21 | GAYARCPNGTRALTVAELRGNAEL |
| Clostridium tetani2 Th | 22 | WVRDIIDDFTNESSQKT |
| Diphtheria Th | 23 | DSETADNLEKTVAALSILPGHGC |
| Plasmodium falciparum Th | 24 | DHEKKHAKMEKASSVFNVVNS |
| Schistosoma mansoni Th | 25 | KWFKTNAPNGVDEKHRH |
| Cholera Toxin Th | 26 | ALNIWDRFDVFCTLGATTGYLKGNS |
| MvF2 Th | 27 | ISEIKGVIVHKIEGI |
| KKKMvF3 Th | 28 | KKKISISEIKGVIVHKIEGILF |
| | | T RT TR T |
| HBsAg1 Th | 29 | KKKLFLLTKLLTLPQSLD |
| | | RRRIKII RII I L IR |
| | | VRVV VV V I V |
| | | F FF FF F V F |
| | | F |
| MvF4 Th (UBITh®3) | 30 | ISISEIKGVIVHKIETILF |
| | | T RT TR |
| HBsAg2 Th | 31 | KKKIITITRIITIPQSLD |
| | | FFLL L ITTI |
| MvF5 Th (UBITh®1) | 32 | ISITEIKGVIVHRIETILF |
| HBsAg3 Th (UBITh®2) | 33 | KKKIITITRIITIITTID |
| Influenza MP1_1 Th | 34 | FVFTLTVPSER |
| Influenza MP1_2 Th | 35 | SGPLKAEIAQRLEDV |
| Influenza NSP1 Th | 36 | DRLRRDQKS |
| EBV BHRF1 Th | 37 | AGLILSLLVICSYLFISRG |
| Clostridium tetani TT1 Th | 38 | QYIKANSKFIGITEL |
| EBV EBNA-1 Th | 39 | PGPLRESIVCYFMVFLQTHI |
| Clostridium tetani TT2 Th | 40 | FNNFTVSFWLRVPKVSASHLE |
| Clostridium tetani TT3 Th | 41 | KFIIKRYTPNNEIDSF |
| Clostridium tetani TT4 Th | 42 | VSIDKFRIFCKALNPK |
| EBV CP Th | 43 | VPGLYSPCRAFFNKEELL |
| HCMV IE1 Th | 44 | DKREMWMACIKELH |
| EBV GP340 Th | 45 | TGHGARTSTEPTTDY |
| EBV BPLF1 Th | 46 | KELKRQYEKKLRQ |
| EBV EBNA-2 Th | 47 | TVFYNIPPMPL |

TABLE 4
Amino Acid Sequences of Myostatin Peptide Immunogen Constructs*

| SEQ | | |
|---|---|---|
| ID | | |
| NO | Description | Sequence |
| 48 | Myostatin construct - | UBITh3-ϵK-CDEHSTESRSC |
| | UBITh3-ϵK- (C272- | |
| | C282, C281→S281), | |
| | cyclized | |
| 49 | Myostatin construct - | CDEHSTESRSC-ϵK-UBITh3 |
| | (C272-C282, | |
| | C281→S281)-ϵK- | |
| | UBITh3, cyclized | |
| 50 | Myostatin construct - | UBITh3-ϵK-CDEHSTESRSC-ϵK-UBITh3 |
| | UBITh3-ϵK- (C272- | |
| | C282, C281→S281)- | |
| | ϵK-UBITh3, cyclized | |
| 51 | Myostatin construct - | UBITh3-ϵK-KKK- |
| | UBITh3-ϵK-KKK- | CLTVDFEAFGWDWIIAPKRYKAC |
| | (P285→C285 to | |
| | N307→C307), cyclized | |
| 52 | Myostatin construct - | CLTVDFEAFGWDWIIAPKRYKAC -KKK- |
| | (P285→C285 to | ϵK-UBITh3 |
| | N307→C307) -KKK - | |
| | ϵK-UBITh3, cyclized | |
| 53 | Myostatin construct - | UBITh3-ϵK-KKK- |
| | UBITh3-ϵK-KKK- | CLTVDFEAFGWDWIIAPKRYKAC -KKK- |
| | (P285→C285 to | ϵK-UBITh3 |
| | N307→C307) -KKK- | |
| | ϵK-UBITh3, cyclized | |
| 54 | Myostatin construct - | UBITh3-ϵK-KKK- |
| | UBITh3-ϵK-KKK- | CKYPHTHLVHQANPRGSAGC |
| | (Q319→C319 to | |
| | p338→C338), cyclized | |
| 55 | Myostatin construct - | CKYPHTHLVHQANPRGSAGC -KKK-ϵK- |
| | (Q319→C319 to | UBITh3 |
| | p338→C338) -KKK - | |
| | ϵK-UBITh3, cyclized | |
| 56 | Myostatin construct - | UBITh3-ϵK-KKK- |
| | UBITh3-ϵK-KKK- | CKYPHTHLVHQANPRGSAGC -KKK-ϵK- |
| | (Q319→C319 to | UBITh3 |
| | p338→C338) -KKK- | |
| | ϵK-UBITh3, cyclized | |
| 57 | Myostatin construct - | UBITh3-ϵK-KKK- |
| | UBITh3-ϵK-KKK- | CINMLYFNGKEQIIYGKIC |
| | (P347→C347 to | |
| | P365→C365), cyclized | |
| 58 | Myostatin construct - | CINMLYFNGKEQIIYGKIC -KKK-ϵK- |
| | (P347→C347 to | UBITh3 |
| | P365→C365) -KKK - | |
| | ϵK-UBITh3, cyclized | |
| 59 | Myostatin construct - | UBITh3-ϵK-KKK- |
| | UBITh3-ϵK-KKK- | CINMLYFNGKEQIIYGKIC - KKK-ϵK- |
| | (P347→C347 to | UBITh3 |
| | P365→C365) -KKK- | |
| | ϵK-UBITh3, cyclized | |
| 60 | Myostatin construct - | UBITh3--ϵK-KKK- |
| | UBITh3-ϵk-kkk-(E314- | CFVFLQKYPHTHLVHQANPRGSAGC |
| | P338 to C314-C338), | |
| | cyclized | |
| 61 | Myostatin construct - | CFVFLQKYPHTHLVHQANPRGSAGC- |
| | (E314-P338 to C314- | KKK-ϵK-UBITh3 |
| | C338)-kkk-ϵk-UBITh3, | |
| | cyclized | |
| 62 | Myostatin construct - | UBITh3--ϵK-KKK- |
| | UBITh3-ϵk-kkk-(E314- | CFVFLQKYPHTHLVHQANPRGSAGC- |
| | P338 to C314-C338)- | KKK-ϵK-UBITh3 |
| | kkk-ϵk-UBITh3, | |
| | cyclized | |
| 63 | Myostatin construct - | UBITh3-ϵK-KKK- |
| | UBITh3-ϵk-kkk-(C313- | CEFVFLQKYPHTHLVHQANPRGSAGC |
| | P338, P338→C338), | |
| | cyclized | |
| 64 | Myostatin construct - | CEFVFLQKYPHTHLVHQANPRGSAGC- |
| | (C313- P338, | KKK-ϵK-UBITh3 |
| | P338→C338)-kkk-ϵk- | |
| | UBITh3, cyclized | |
| 65 | Myostatin construct - | UBITh3--ϵK-KKK- |
| | UBITh3-ϵk-kkk-(C313- | CEFVFLQKYPHTHLVHQANPRGSAGC- |
| | P338, P338→C338)- | KKK-ϵK-UBITh3 |
| | kkk-ϵk-UBITh3, | |
| | cyclized | |
| 66 | Myostatin construct - | UBITh3--ϵK-KKK- |
| | UBITh3-ϵk-kkk-(G311- | CESEFVFLQKYPHTHLVHQANPRGSAGC |
| | P338 to C311-C338, | |
| | C47→S47), cyclized | |
| 67 | Myostatin construct - | CESEFVFLQKYPHTHLVHQANPRGSAGC- |
| | (G311-P338 to C311- | KKK-ϵK-UBITh3 |
| | C338, C47→S47)-kkk- | |
| | ϵk-UBITh3, cyclized | |
| 68 | Myostatin construct - | UBITh3-ϵK-KKK- |
| | UBITh3-ϵk-kkk-(G311- | CESEFVFLQKYPHTHLVHQANPRGSAGC- |
| | P338 to C311-C338, | KKK-ϵK-UBITh3 |
| | C47→S47)-kkk-ϵk- | |
| | UBITh3, cyclized | |
| 69 | Myostatin construct - | UBITh3-ϵk-kkk-CSGESEFVFLQC |
| | UBITh3-ϵk-kkk-(C309- | |
| | K320, K320→C320, | |
| | C313→S313), cyclized | |
| 70 | Myostatin construct - | CSGESEFVFLQC-KKK-ϵK-UBITh3 |
| | (C309-K320, | |
| | K320→C320, | |
| | C313→S313)-kkk-ϵk- | |
| | UBITh3, cyclized | |
| 71 | Myostatin construct - | UBITh3-ϵk-kkk-CSGESEFVFLQC-KKK-ϵK- |
| | UBITh3-ϵk-kkk-(C309- | UBITh3 |
| | K320, K320→C320, | |
| | C313→S313) | |
| 120 | Myostatin construct - | EFVFLQKYPH-KKK-ϵK-UBITh1 |
| | (E314-H323)-kkk-ϵk- | |
| | UBITh1 | |
| 121 | Myostatin construct - | ECEFVFCQKY-KKK-ϵK-UBITh1 |
| | (E312-Y321, | |
| | L318→C318)-kkk-ϵk- | |
| | UBITh1, cyclized | |
| 122 | Myostatin construct - | QKYPHTHLVHQANPRGSAGP-KKK-ϵK- |
| | (Q319-P338)-kkk-ϵk- | UBITh1 |
| | UBITh1 | |
| 123 | Myostatin construct - | CKYPHTHLVHQANPRGSAGC-KKK-ϵK- |
| | (Q319-P338, | UBITh1 |
| | Q319→C319, | |
| | P338→C338)-kkk-ϵk- | |
| | UBITh1, cyclized | |
| 124 | Myostatin construct - | ECEFVFCQKYPHTHLVH-KKK-ϵK-UBITh1 |
| | (E314-H328, | |
| | L318→C318)-kkk-ϵk- | |
| | UBITh1, cyclized | |
| 125 | Myostatin construct - | EFVFLQKYPHTHLVHQANPRGSAGP-KKK- |
| | (E314-P338)-kkk-ϵk- | ϵK-UBITh1 |
| | UBITh1 | |
| 126 | Myostatin construct - | CEFVFLQKYPHTHLVHQANPRGSAGPC- |
| | (C313-C339)-kkk-ϵk- | KKK-ϵK-UBITh1 |
| | UBITh1, cyclized | |
| 127 | Myostatin construct - | EFVFLQKYPHTHLVHQA-KKK-ϵK-UBITh1 |
| | (E314-A330)-kkk-ϵk- | |
| | UBITh1 | |
| 128 | Myostatin construct - | Ahx-KYPHTHLVHQANPRGSAGE-KKK-ϵK- |
| | (Q319-P338, | UBITh1 |
| | Q319→Ahx319, | |
| | P338→E338)-kkk-ϵk- | |
| | UBITh1, cyclized | |
| 129 | Myostatin construct - | QKYPCTHLVHQANPRGSC-KKK-ϵK- |
| | (Q319-A336, | UBITh1 |
| | H323→C323, | |
| | A336→C336)-kkk-ϵk- | |
| | UBITh1, cyclized | |
| 130 | Myostatin construct - | QKYPDTHLVHQANPRGSDap-KKK-ϵK- |
| | (Q319-A336, | UBITh1 |
| | H323→D323, | |
| | A336→Dap336)-kkk- | |
| | ϵk-UBITh1, cyclized | |
| 131 | Myostatin construct - | PHTHL VHQANPRGSAE-KKK-ϵK-UBITh1 |
| | (P322-G337, | |
| | G337→E337)-kkk-ϵk- | |
| | UBITh1, cyclized | |
| 132 | Myostatin construct - | EFVFLQKY-KKK-ϵK-UBITh1 |
| | (E314-Y321)-kkk-ϵk- | |
| | UBITh1, cyclized | |
| 133 | Myostatin construct - | EFVFLQKYPCTHLVHQANPRGSC-KKK-ϵK- |
| | (E314-A336, | UBITh1 |
| | H323→C323, | |
| | A336→C336)-kkk-ϵk- | |
| | UBITh1, cyclized | |
| 134 | Myostatin construct - | DKYPHTHLVHQANPRGSAGDap-KKK-ϵK- |
| | (Q319-P338, | UBITh1 |
| | Q319→D319, | |
| | P338→Dap338)-kkk- | |
| | ϵk-UBITh1, cyclized | |
| 167 | Myostatin construct - | YKANYSSGESEFEFLDab-KKK-ϵK-UBITh1 |
| | (Y304-Q319, | |
| | C309→S309, | |
| | C313→S313, | |
| | V316→E316, | |
| | Q319→Dab319)-kkk- | |
| | ϵk-UBITh1, cyclized | |
| 168 | Myostatin construct - | YKANYSSGESEFVFLQ-KKK-ϵK-UBITh1 |
| | (Y304-Q319, | |
| | C309→S309, | |
| | C313→S313)-kkk-ϵk- | |
| | UBITh1 | |
| 169 | Myostatin construct - | QDab YPHTHLEHQANPR-KKK-ϵK-UBITh1 |
| | (Q319-R333, | |
| | K320→Dab320, | |
| | V327→ E327)-kkk-ϵk- | |
| | UBITh1, cyclized | |
| 170 | Myostatin construct - | QKYPHTHLVHQ-KKK-ϵK-UBITh1 |
| | (Q319-Q329)-kkk-ϵk- | |
| | UBITh1 | |
| 171 | Myostatin construct - | HTHLVHQANPR-KKK-ϵK-UBITh1 |
| | (H323-R333)-kkk-ϵk- | |
| | UBITh1 | |
| 172 | Myostatin construct - | QKYPHTHLCHQANPRC-KKK-ϵK-UBITh1 |
| | (Q319-G334, | |
| | V327→C327, G334→ | |
| | C334)-kkk-ϵk-UBITh1, | |
| | cyclized | |
| 173 | Myostatin construct - | YKANYSSGESEFVFLQKYPHT-KKK-ϵK- |
| | (Y304-T324, | UBITh1 |
| | C309→S309, | |
| | C313→S313)-kkk-ϵk- | |
| | UBITh1 | |
| 174 | Myostatin construct - | YSSGESEFVFLQKYPHTHLV-KKK-ϵK- |
| | (Y308-V327, | UBITh1 |
| | C309→S309, | |
| | C313→S313)-kkk-ϵk- | |
| | UBITh1 | |
| 175 | Myostatin construct - | YKANYSSGESEFEFLDabKYPHT-KKK-ϵK- |
| | (Y304-T324, | UBITh1 |
| | C309→S309, | |
| | C313→S313, | |
| | V316→E316, | |
| | Q319→Dab319)-kkk- | |
| | ϵk-UBITh1 | |
| 176 | Myostatin construct - | YSSGESEFEFLDabKYEHTHLVOrn-KKK- |
| | (Y308-H328, | ϵK-UBITh1 |
| | C309→S309, | |
| | C313→S313, | |
| | V316→E316, | |
| | Q319→Dab319, | |
| | P322→E322, | |
| | H328→Orn328)-kkk- | |
| | ϵk-UBITh1 | |
| 177 | Myostatin construct - | LLPKAPPLRELID-KKK-ϵK-UBITh1 |
| | (L80-D92)-kkk-ϵk- | |
| | UBITh1 | |
| 178 | Myostatin construct - | QDabYPHTHLE-KKK-ϵK-UBITh1 |
| | (Q319-E327, | |
| | K320→Dab320, | |
| | V327→ E327)-kkk-ϵk- | |
| | UBITh1, cyclized | |
| 179 | Myostatin construct - | QDabYPHTHLEH-KKK-ϵK-UBITh2 |
| | (Q319-H328, | |
| | K320→Dab320, | |
| | V327→ E327)-kkk-ϵk- | |
| | UBITh2, cyclized | |
| 180 | Myostatin construct - | LCELICQYDVQR-KKK-ϵK-UBITh1 |
| | (L87-R98, | |
| | R88→C88, D92→C92)- | |
| | kkk-ϵk-UBITh1 | |
| 181 | Myostatin construct - | VQRDDSSDGSLEDDDYH-KKK-ϵK-UBITh1 |
| | (V96-H112)-kkk-ϵk- | |
| | UBITh1 | |
| 182 | Myostatin construct - | IITMPTESDFLM-KKK-ϵK-UBITh1 |
| | (I118-M129)-kkk-ϵk- | |
| | UBITh1 | |
| 183 | Myostatin construct - | LNPFLEVKVTDT-KKK-ϵK-UBITh1 |
| | (L249-T260)-kkk-ϵk- | |
| | UBITh1 | |
| 184 | Myostatin construct - | VTDTPKSRRDFGL-KKK-ϵK-UBITh1 |
| | (V257-L270)-kkk-ϵk- | |
| | UBITh1 | |
| 185 | Myostatin construct - | FLEVKVTDTPK-KKK-ϵK-UBITh1 |
| | (F252-K262)-kkk-ϵk- | |
| | UBITh1 | |
| 186 | Myostatin construct - | LAVTFPGPGEDGL-KKK-ϵK-UBITh1 |
| | (L237-L249)-kkk-ϵk- | |
| | UBITh1 | |
| 188 | Myostatin construct - | TVFVQILRLIKPM-KKK-ϵK-UBITh1 |
| | (T168-M180)-kkk-ϵk- | |
| | UBITh1 | |

*Note:
“Ahx” is 6-aminohexanoic acid. “Dap” is L-2,3 diaminopropionic acid (CAS 1482-97-9). “Dab” is L-2,4 diaminobutyric acid (CAS 1883-09-6). “Orn” is L-Ornithine (CAS 3184-13-2).
*For each peptide immunogen construct listed, B cell epitope peptides are acetylated on the N-terminus and have an amide on the C-terminus (except for the peptides that are cyclized using the N-terminus amino group, such as peptides of SEQ ID NO: 103 (Ahx-KYPHTHLVHQANPRGSAGE) and SEQ ID NO: 106 (PHTHLVHQANPRGSAE)).
Note:
for each peptide immunogen construct listed in this table, the indication of a mixed T cell epitope (e.g., UBITh3) is to be considered as including the presence of any form of the T cell epitope peptide, including any one or more (e.g., all) of the possible combinations. Accordingly, the sequence of UBITh3 can be considered as ISIXEIXXVIVXXIETILF (SEQ ID NO: 193), wherein the 1st X is S or T, the 2nd X is K or R, the 3rd X is G or T, the 4th X is H or T, and the 5th X is K or R, and the selections are independent of one another. If more than one mixed T cell epitope (e.g., UBITh3) is present, then each is to be considered as independently including any one or more (e.g., all) of the possible combinations as described herein. The same applies for other mixed T cell epitopes. See Table 3 for details. In some embodiments, the UBITh3 peptide is replaced with the UBITh1 or UBITh2 peptide. This note also applies to Table 5.

TABLE 5
Amino Acid Sequences of Activin A Peptide Immunogen Constructs*

| SEQ | | |
|---|---|---|
| ID | | |
| NO | Description | Sequence |
| 72 | Activin A peptide - | UBITh3-ϵK-CDGKVNISC |
| | epitope design on loop | |
| | 1: | |
| | aa314-322, | |
| | C321→S321, | |
| | cyclized. (4-12) | |
| 73 | Activin A peptide - | CDGKVNISC-ϵK-UBITh3 |
| | epitope design on loop | |
| | 1: | |
| | aa314-322, | |
| | C321→S321, | |
| | cyclized. (4-12) | |
| 74 | Activin A peptide - | UBITh3-ϵK-CDGKVNISC-ϵK-UBITh3 |
| | epitope design on loop | |
| | 1: | |
| | aa314-322, | |
| | C321→S321, | |
| | cyclized. (4-12) | |
| 75 | Activin A peptide - | UBITh3-ϵK-CFKDIGWNDWIIAPSC |
| | epitope design on loop | |
| | 2: | |
| | aa329-344, | |
| | S329→C329, | |
| | G344→C344, | |
| | cyclized. (19-34) | |
| 76 | Activin A peptide - | CFKDIGWNDWIIAPSC-ϵK-UBITh3 |
| | epitope design on loop | |
| | 2: | |
| | aa329-344, | |
| | S329→C329, | |
| | G344→C344, | |
| | cyclized. (19-34) | |
| 77 | Activin A peptide - | UBITh3-ϵK-CFKDIGWNDWIIAPSC-ϵK- |
| | epitope design on loop | UBITh3 |
| | 2: | |
| | aa329-344, | |
| | S329→C329, | |
| | G344→C344, | |
| | cyclized. (19-34) | |
| 78 | Activin A peptide - | UBITh3-ϵK- |
| | epitope design on loop | CSLSFHSTVINHYRMRGHSPFANLKSC |
| | 3A: | |
| | aa364-390, | |
| | S364→C364, | |
| | cyclized. (54-80) | |
| 79 | Activin A peptide - | CSLSFHSTVINHYRMRGHSPFANLKSC-ϵK- |
| | epitope design on loop | UBITh3 |
| | 3A: | |
| | aa364-390, | |
| | S364→C364, | |
| | cyclized. (54-80) | |
| 80 | Activin A peptide - | UBITh3-ϵK- |
| | epitope design on loop | CSLSFHSTVINHYRMRGHSPFANLKSC-ϵK- |
| | 3A: | UBITh3 |
| | aa364-390, | |
| | S364→C364, | |
| | cyclized. (54-80) | |
| 81 | Activin A peptide - | UBITh3-ϵK-CPSHIAGTSGSSLSFHC |
| | epitope design on loop | |
| | 3B: | |
| | aa354-370, | |
| | S370 → C370, cyclized. | |
| | (44-60) | |
| 82 | Activin A peptide - | CPSHIAGTSGSSLSFHC-ϵK-UBITh3 |
| | epitope design on loop | |
| | 3B: | |
| | aa354-370, | |
| | S370 → C370, cyclized. | |
| | (44-60) | |
| 83 | Activin A peptide - | UBITh3-ϵK-CPSHIAGTSGSSLSFHC-ϵK- |
| | epitope design on loop | UBITh3 |
| | 3B: | |
| | aa354-370, | |
| | S370 → C370, cyclized. | |
| | (44-60) | |
| 84 | Activin A peptide - | UBITh3-ϵK-CTVINHYRMRGHSPFANLKSC |
| | epitope design on loop | |
| | 3C: | |
| | aa370-390, S370 → | |
| | C370, cyclized. (64-80) | |
| 85 | Activin A peptide - | CTVINHYRMRGHSPFANLKSC-ϵK-UBITh3 |
| | epitope design on loop | |
| | 3C: | |
| | aa370-390, S370 → | |
| | C370, cyclized. (64-80) | |
| 86 | Activin A peptide - | UBITh3-ϵK-CTVINHYRMRGHSPFANLKSC- |
| | epitope design on loop | ϵK-UBITh3 |
| | 3C: | |
| | aa370-390, S370 → | |
| | C370, cyclized. (64-80) | |
| 87 | Activin A peptide - | UBITh3-ϵK-CSMLYYDDGQNIIKKDC |
| | epitope design on loop | |
| | 4: | |
| | aa399-415, | |
| | M399→C399, | |
| | I415→C415, | |
| | cyclized. (89-105) | |
| 88 | Activin A peptide - | CSMLYYDDGQNIIKKDC-ϵK-UBITh3 |
| | epitope design on loop | |
| | 4: | |
| | aa399-415, | |
| | M399→C399, | |
| | I415→C415, | |
| | cyclized. (89-105) | |
| 89 | Activin A peptide - | UBITh3-ϵK-CSMLYYDDGQNIIKKDC-ϵK- |
| | epitope design on loop | UBITh3 |
| | 4: | |
| | aa399-415, | |
| | M399→C399, | |
| | I415→C415, | |
| | cyclized. (89-105) | |
| 135 | Activin A construct - | DIGCNDWIIAC-KKK-ϵK-UBITh1 |
| | (D332-P342, | |
| | W335→C335, | |
| | P342→C342)-kkk-ϵk- | |
| | UBITh1, cyclized. | |
| 136 | Activin A construct - | WNDWIIAPSG-KKK-ϵK-UBITh1 |
| | (W335-G344)-kkk-ϵk- | |
| | UBITh1 | |
| 137 | Activin A construct - | STVINHYRMR-KKKϵ-K-UBITh1 |
| | (S370-R379)-kkk-ϵk- | |
| | UBITh1 | |
| 138 | Activin A construct - | HYRMRGHSPF-KKK-ϵK-UBITh1 |
| | (H375-F384)-kkk-ϵk- | |
| | UBITh1 | |
| 139 | Activin A construct - | HSPFANLKSS-KKK-ϵK-UBITh1 |
| | (H381-C390, | |
| | C390→S390)-kkk-ϵk- | |
| | UBITh1 | |
| 140 | Activin A construct - | FKDIGWNDW-KKK-ϵK-UBITh1 |
| | (F330-W338)-kkk-ϵk- | |
| | UBITh1 | |
| 141 | Activin A construct - | STVINHYRMRGHSPFANLKSS-KKK-ϵK- |
| | (S370-C390, | UBITh1 |
| | C390→S390)-kkk-ϵk- | |
| | UBITh1 | |
| 142 | Activin A construct - | CTVINHYRMRGHSPFANLKSC-KKK-ϵK- |
| | (S370-C390, | UBITh1 |
| | S370→C370)-kkk-ϵk- | |
| | UBITh1, cyclized. | |
| 143 | Activin A construct - | CFKDIGWNDWIIAPSC-KKK-ϵK-UBITh1 |
| | (S329-G344, | |
| | S329→C329, | |
| | G344→C344)-kkk-ϵk- | |
| | UBITh1, cyclized. | |
| 144 | Activin A construct - | SFKDIGWNDWIIAPSG-KKK-ϵK-UBITh1 |
| | (S329-G344)-kkk-ϵk- | |
| | UBITh1 | |
| 190 | Activin A construct - | YRMRGHSPFA-KKK-ϵK-UBITh1 |
| | (Y376-A385)-kkk-ϵk- | |
| | UBITh1 | |
| 191 | Activin A construct - | HYRMRGHSPFAN-KKK-ϵK-UBITh1 |
| | (H375-N386)-kkk-ϵk- | |
| | UBITh1 | |

*For each peptide immunogen construct listed, B cell epitope peptides are acetylated on the N-terminus and have an amide on the C-terminus (except for the peptides that are cyclized using the N-terminus amino group, such as peptides of SEQ ID NO: 103 (Ahx-KYPHTHLVHQANPRGSAGE) and SEQ ID NO: 106 (PHTHLVHQANPRGSAE)).

Accordingly, the invention includes each of the constructs listed in Table 4, above, but with any one of the Th epitopes listed in Table 3 inserted in place of UBITh1, UBITh2, or UBITh3. Furthermore, any of the linkers described herein can be utilized in place of the linkers set forth in Table 4. The invention also includes each of the constructs listed in Table 5, above, but with any one of the Th epitopes listed in Table 3 inserted in place of UBITh1 or UBITh3. Furthermore, any of the linkers described herein can be utilized in place of the linkers set forth in Table 5.

Exemplary linker sequences are as follows:

SEQ ID NO: 90
Pro-Pro-Xaa-Pro-Xaa-Pro
Xaa = any amino acid, e.g., aspartic acid
SEQ ID NO: 91
ϵ-N-Lys-Lys-Lys-Lys

The disclosure is not limited to the exemplary embodiments and applications presented herein or to the manner in which the exemplary embodiments and applications operate or are described herein. Other embodiments are within the scope of the claims.

1. A peptide immunogen construct comprising a myostatin B cell epitope, a heterologous T cell (Th) epitope, and an optional heterologous spacer, wherein the B cell epitope is covalently linked to the Th epitope directly or through the optional heterologous spacer; wherein the myostatin B cell epitope comprises about 7 to about 30 contiguous amino acids of SEQ ID NO: 1 or SEQ ID NO: 2 or a variant thereof.

2. The peptide immunogen construct of claim 1, wherein
(i) the myostatin B cell epitope comprises a variant of an 7-30 amino acid fragment of SEQ ID NO: 1 or SEQ ID NO: 2, and the variant comprises 1, 2, 3, 4, 5, or 6 amino acid substitutions, deletions, or insertions as compared to the corresponding sequence of the 7-30 amino acid fragment of SEQ ID NO: 1 or SEQ ID NO: 2;
(ii) the myostatin B cell epitope comprises up to 3, or up to 6, amino acid substitutions as compared to the corresponding sequence of the 7-30 amino acid fragment of SEQ ID NO: 1 or SEQ ID NO: 2;
(iii) the myostatin B cell epitope comprises one or more (optionally 1, 2, or 3) amino acid substitutions as compared to the sequence of SEQ ID NO: 1 or SEQ ID NO: 2 to facilitate or enable cyclization of the sequence comprising the myostatin B cell epitope, optionally wherein the myostatin B cell epitope is cyclized; and/or
(iv) the myostatin B cell epitope comprises or consists of an amino acid sequence within loop 1, loop 2, loop 3, or loop 4 of myostatin, or a variant thereof.

3-5. (canceled)

6. The peptide immunogen construct of claim 1, wherein the myostatin B cell epitope comprises or consists of the amino acid sequence of any one of SEQ ID NOs: 3-10, 95-109, 145-164, and 166 or a variant thereof; optionally wherein the myostatin B cell epitope comprises or consists of:
(i) the amino acid sequence of SEQ ID NO: 5, 97, 98, 101, 105, 146, 147, 151, 152, 153, 154, 155, 157, 159, 161, 163, 164, or 166;
(ii) the amino acid sequence of SEQ ID NO: 5, 97, 98, 101, or 105; or
(iii) the amino acid sequence of SEQ ID NOs: 146, 147, 151, 152, 154, 155, 157, 159, 161, 163, 164, or 166.

7-9. (canceled)

10. The peptide immunogen construct of claim 1, wherein the Th epitope is selected from any one of SEQ ID NOs: 19-47, optionally wherein the Th epitope is of SEQ ID NO: 30, 32, or 33.

11. (canceled)

12. The peptide immunogen construct of claim 1, wherein the myostatin B cell epitope and the Th epitope are linked to one another by the heterologous spacer; optionally wherein
(i) the heterologous spacer comprises or consists of one or more amino acids, which are optionally selected from: Lys-, Gly-, Lys-Lys-Lys-, Lys-Lys-Lys-ε-Lys (SEQ ID NO: 192), (α, ε-N)Lys, ε-N-Lys-Lys-Lys-Lys (SEQ ID NO: 91), and Pro-Pro-Xaa-Pro-Xaa-Pro, wherein Xaa is any amino acid or is aspartic acid (SEO ID NO: 90); or
(ii) the heterologous spacer is Lys-Lys-Lys-, Lys-Lys-Lys-ε-Lys (SEQ ID NO: 192), (α, ε-N)Lys, or ε-N-Lys-Lys-Lys-Lys (SEQ ID NO: 91).

13-14. (canceled)

15. The peptide immunogen construct of claim 1, wherein:
(i) (a) the myostatin B cell epitope is located N-terminal to the Th epitope;
(b) the Th epitope is located N-terminal to the myostatin B cell epitope;
(c) the myostatin B cell epitope is flanked with a Th epitope on both N-terminal and C-terminal ends;
(d) the myostatin B cell epitope is acetylated on the N-terminus or cyclized using the N-terminus amino group, and have an amide on the C-terminus;
(e) any of (a)-(d) wherein the myostatin B cell epitope(s) is linked to the Th epitope(s) by direct covalent linkage; or
(f) any one of (a)-(d) wherein the myostatin B cell epitope(s) is linked to the Th epitope(s) by the heterologous spacer; and/or
(ii) the peptide immunogen construct is of following formula: 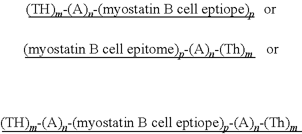
wherein
Th is the T helper epitope;
A is the heterologous spacer, which optionally comprises an amino acid;
m is from 1 to about 4;
p is from 1 to about 4;
and
n is from 0 to about 10, wherein optionally the C-terminus of the peptide immunogen construct comprises α-COOH or α-CONH₂ of an amino acid; optionally wherein m is 1, p is 1 or 2, and n is 1.

16. (canceled)

17. The peptide immunogen construct of claim 1, wherein the peptide immunogen construct comprises or consists of the sequence of any one of SEQ ID NOs: 48-71, 120-134, 167-186 and 188 or a variant thereof; optionally wherein the peptide immunogen construct comprises or consists of:
(i) the sequence of SEQ ID NO: 54, 55, 56, 122, 123, 126, 130, 168, 169, 173, 174, 175, 176, 177, 178, 179, 181, 183, 185, or 186;
(ii) the sequence of SEQ ID NO: 54, 55, 56, 122, 123, 126 or 130; or
(iii) the sequence of SEQ ID NO: 168, 169, 173, 174, 176, 177, 179, 181, 183, 185, or 186.

18-20. (canceled)

21. A peptide immunogen construct comprising an activin A B cell epitope, a heterologous T cell (Th) epitope, and an optional heterologous spacer, wherein the B cell epitope is covalently linked to the Th epitope directly or through the optional heterologous spacer, wherein the activin A B cell epitope comprises about 7 to about 30 contiguous amino acids of SEQ ID NO: 12 or a variant thereof.

22. The peptide immunogen construct of claim 21, wherein
(i) the activin A B cell epitope comprises a variant of a 7-30 amino acid fragment of SEQ ID NO: 12, and the variant comprises 1, 2, 3, 4, 5, or 6 amino acid substitutions, deletions, or insertions as compared to the corresponding sequence of the 7-30 amino acid fragment of SEQ ID NO: 12;
(ii) the activin A B cell epitope comprises up to 3, or up to 6, amino acid substitutions as compared to the corresponding sequence of the 7-30 amino acid fragment of SEQ ID NO: 12
(iii) the activin A B cell epitope comprises one or more (optionally 1, 2, or 3) amino acid substitutions as compared to the sequence of SEQ ID NO: 12 to facilitate or enable cyclization of the sequence comprising the activin A B cell epitope, optionally wherein the activin A B cell epitope is cyclized; and/or
(iv) the activin A B cell epitope comprises or consists of an amino acid sequence within loop 1, loop 2, loop 3, or loop 4 of activin A, or a variant thereof.

23-25. (canceled)

26. The peptide immunogen construct of claim 21, wherein the activin A B cell epitope comprises or consists of the amino acid sequence of any one of SEQ ID NOs: 13-18, 110-119, 187, and 189 or a variant thereof; optionally wherein the activin A B cell epitope comprises or consists of the amino acid sequence of SEQ ID NO: 14, 15, 17, 111, 116, 118, 119, or 187.

27. (canceled)

28. The peptide immunogen construct of claim 21, wherein the Th epitope is selected from any one of SEQ ID NOs: 19-47, optionally wherein the Th epitope comprises or consists of the sequence of SEQ ID NO: 30, 32, or 33.

29. (canceled)

30. The peptide immunogen construct of claim 21, wherein the activin A B cell epitope and the Th epitope are linked to one another by the heterologous spacer; optionally wherein
(i) the heterologous spacer comprises or consists of one or more amino acids, which are optionally selected from: Lys-, Gly-, Lys-Lys-Lys-, Lys-Lys-Lys-ε-Lys (SEO ID NO: 192), (α, ε-N)Lys, ε-N-Lys-Lys-Lys-Lys (SEQ ID NO: 91), and Pro-Pro-Xaa-Pro-Xaa-Pro, wherein Xaa is any amino acid or is aspartic acid (SEO ID NO: 90); or
(ii) the heterologous spacer is Lys-Lys-Lys-, Lys-Lys-Lys-ε-Lys (SEQ ID NO: 192), (α, ε-N)Lys, or ε-N-Lys-Lys-Lys-Lys (SEQ ID NO: 91).

31-32. (canceled)

33. The peptide immunogen construct of claim 21, wherein:
(i) (a) the activin A B cell epitope is located N-terminal to the Th epitope;
(b) the Th epitope is located N-terminal to the activin A B cell epitope;
(c) the activin A B cell epitope is flanked with a Th epitope on both N-terminal and C-terminal ends;
(d) the activin A B cell epitope is acetylated on the N-terminus or cyclized using the N-terminus amino group, and have an amide on the C-terminus;
(e) any of (a)-(d) wherein the activin A B cell epitope(s) is linked to the Th epitope(s) by direct covalent linkage; or
(f) any one of (a)-(d) wherein the activin A B cell epitope(s) is linked to the Th epitope(s) by the heterologous spacer; and/or
(ii) the peptide immunogen construct is of following formula: 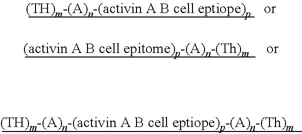
wherein
Th is the T helper epitope;
A is the heterologous spacer, which optionally comprises an amino acid;
m is from 1 to about 4;
p is from 1 to about 4;
and
n is from 0 to about 10, wherein optionally the C-terminus of the peptide immunogen construct comprises α-COOH or α-CONH₂ of an amino acid; optionally m is 1, p is 1 or 2, and n is 1.

34. (canceled)

35. The peptide immunogen construct of claim 21, wherein the peptide immunogen construct comprises or consists of the sequence of any one of SEQ ID NOs: 72-89, 135-144, and 190-191 or a variant thereof; optionally wherein the peptide immunogen construct comprises or consists of the sequence of SEO ID NOs: 75, 76, 78, 79, 85, 136, 138, 139, 140, 141, 142, 143, 144, or 190; and further optionally wherein the construct comprises or consists of SEQ ID NOs: 75, 76, 78, 79, 85, 136, 141, 143, 144, or 190.

36. (canceled)

37. A peptide immunogen construct comprising:
(i) two or more B cell epitopes selected from:
a myostatin B cell epitope comprising about 7 to about 30 contiguous amino acids of SEQ ID NO: 1 or SEQ ID NO: 2 or a variant thereof, and
an activin A B cell epitope comprising about 7 to about 30 contiguous amino acids of SEQ ID NO: 12 or a variant thereof;
(ii) a heterologous T cell (Th) epitope, and
(iii) optionally, one or more heterologous spacers and/or linkers,
wherein the myostatin and activin A B cell epitopes are covalently linked to the Th epitope directly or through the one or more heterologous spacers and/or linkers.

38. The peptide immunogen construct of claim 37, wherein the two or more B cell epitopes are two to four B cell epitopes, optionally wherein the two or more B cell epitopes are two or three B cell epitopes.

39. The peptide immunogen construct of claim 37, wherein
(i) the two or more B cell epitopes are two or more of the myostatin B cell epitopes, optionally wherein the two or more B cell epitopes do not comprise an activin A B cell epitope;
(ii) the two or more B cell epitopes are two or more of the activin A B cell epitopes, optionally wherein the two or more B cell epitopes do not comprise a myostatin B cell epitope; or
(iii) the two or more B cell epitopes comprise at least one myostatin B cell epitope and at least one activin A B cell epitope.

40-41. (canceled)

42. A peptide immunogen construct comprising:
(i) a myostatin B cell epitope, wherein the myostatin B cell epitope comprises about 7 to about 30 contiguous amino acids of SEQ ID NO: 1 or SEQ ID NO: 2 or a variant thereof;
(ii) an activin A B cell epitope, wherein the activin A B cell epitope comprises about 7 to about 30 contiguous amino acids of SEQ ID NO: 12 or a variant thereof;
(iii) a heterologous T cell (Th) epitope, and
(iv) one or more heterologous spacers and/or linkers,
wherein the myostatin and activin A B cell epitopes are covalently linked to the Th epitope through the one or more heterologous spacers and/or linkers.

43. The peptide immunogen construct of claim 37, wherein the one or more heterologous spacers and/or linkers comprise a branched amino acid; optionally wherein:
(i) the branched amino acid is a diamino acid and the B cell epitopes are attached to the N-terminus of the Th epitope, optionally wherein the diamino acid is lysine; or
(ii) the branched amino acid is a dicarboxylic acid and the B cell epitopes are attached to the C-terminus of the Th epitope, optionally wherein the dicarboxylic acid is glutamic acid.

44-45. (canceled)

46. The peptide immunogen construct of claim 43, wherein
(i) the branched amino acid is linked to each of the B cell epitopes by a linker, optionally wherein the linker is an amino acid peg derivative; optionally wherein the peg derivative is 8-amino-3,6-dioxaoctanoic acid or 2-[2-(2-aminoethoxy)ethoxy]acetic acid (“AeeA”); and/or
(ii) the branched amino acid is covalently linked to the Th epitope by a heterologous spacer, optionally wherein the heterologous spacer is a polylysine spacer, optionally wherein the polylysine spacer comprises or consists of 3 or 4 lysines, further optionally wherein the heterologous spacer is Lys-Lys-Lys-, Lys-Lys-Lys-ε-Lys (SEO ID NO: 192) (α, ε-N)Lys, or ε-N-Lys-Lys-lys-Lys (SEQ ID NO: 91).

47-50. (canceled)

51. The peptide immunogen construct of claim 42, wherein
(i) the myostatin B cell epitope comprises or consists of the amino acid sequence of any one of SEQ ID NOs: 3-10, 95-109, 145-164, and 166 or a variant thereof; optionally wherein the myostatin B cell epitope is of the amino acid sequence of SEQ ID NO: 5, 97, 98, 101, 105, 146, 147, 151-155, 157, 159, 161, 163, 164, or 166;
(ii) the activin A B cell epitope comprises or consists of the amino acid sequence of any one of SEQ ID NOs: 13-18, 110-119, 187, and 189 or a variant thereof; optionally wherein the activin A B cell epitope is of the amino acid sequence of SEQ ID NO: 14, 15, 17, 111, 116, 118, 119, or 187; and/or
(iii) the myostatin B cell epitope and/or activin A B cell epitope is cyclized;
optionally wherein the myostatin B cell epitope is of the amino acid sequence of SEQ ID NO: 97 or a variant thereof, and/or the activin A B cell epitope is of the amino acid sequence of SEQ ID NO: 116 or a variant thereof.

52-54. (canceled)

55. The peptide immunogen construct of claim 37, wherein the Th epitope is selected from any one of SEQ ID NOs: 19-47, optionally wherein the Th epitope is of the amino acid sequence of SEQ ID NO: 30, 32, or 33, further optionally wherein the Th epitope is of the amino acid sequence of SEQ ID NO: 32.

56. The peptide immunogen construct of claim 42, which comprises or consists of Ac-STVINHYRMRGHSPFANLKSSAeeA(Ac-QKYPHTHLVHQANPRGSAGPAeeA)K-KKKrK-Ubith1 (SEQ ID NO: 165).

57. A nucleic acid molecule encoding a peptide immunogen construct of claim 37.

58. A vector comprising the nucleic acid molecule of claim 57.

59. An isolated antibody or an epitope-binding fragment thereof that specifically binds to and/or is elicited by the myostatin B cell epitope and/or the activin A B cell epitope of the peptide immunogen construct of claim 37.

60. A composition comprising: (a) one or more peptide immunogen constructs of claim 37, and (b) a pharmaceutically acceptable carrier, diluent, or adjuvant; optionally wherein the composition comprises (i) an adjuvant that is a mineral salt of aluminum, which optionally is selected from Al(OH)₃ and AlPO₄, and/or (ii) a CpG oligonucleotide, optionally a GpG1 oligonucleotide.

61-62. (canceled)

63. A method of treating or preventing the development of muscle wasting in a subject, the method comprising administering to the subject an effective amount of an immunotherapy targeting myostatin and/or activin A, wherein the immunotherapy comprises a peptide immunogen construct of claim 37.

64. A method of treating, preventing, reducing, inhibiting, or slowing the development of one or more symptom of a muscle wasting disease in a subject, the method comprising administering to the subject an effective amount of an immunotherapy targeting myostatin and/or activin A, wherein the immunotherapy comprises a peptide immunogen construct of claim 37.

65. A method of inducing an immune response to myostatin and/or activin A in a subject, the method comprising administering to the subject an effective amount of an immunotherapy targeting myostatin and/or activin A, wherein the immunotherapy comprises a peptide immunogen construct of claim 37.

66. A method of treating or preventing insulin resistance in a subject, the method comprising administering to the subject an effective amount of an immunotherapy targeting myostatin and/or activin A, wherein the immunotherapy comprises a peptide immunogen construct of claim 37; optionally wherein the subject has or is at risk of developing insulin resistance, type II diabetes, elevated A1C levels, pre-diabetes, metabolic syndrome, hypertension, hyperglycemia, hypercholesterolemia, high triglycerides, overweight, or obesity.

67. The method of claim 63, wherein
(i) the subject has or is at risk of developing muscle wasting due to an inherited or genetic cause, inflammation, infection, injury, systemic disease, toxin, or other environmental exposure;
(ii) the subject has or is at risk of developing muscle wasting due to an inherited or genetic myopathy, which is optionally selected from muscular dystrophy (e.g., Duchenne Muscular Dystrophy (DMD) and Becker Muscular Dystrophy (BMD), myotonic dystrophy (e.g., DM1 and DM2), facioscapulohumeral muscular dystrophy, oculopharyngeal muscular dystrophy, and limb girdle muscular dystrophy), congenital myopathy (e.g., nemaline myopathy and central core myopathy), metabolic myopathy (e.g., acid maltase or acid alpha-1,4-glucosidase deficiency (Pompe's disease), glycogen storage disorders 3-11, carnitine deficiency, fatty acid oxidation defects, and carnitine palmitoyl transferase deficiency), and mitochondrial myopathy;
(iii) the subject has or is at risk of developing muscle wasting due to an autoimmune or inflammatory myopathy (i.e., myositis), which is optionally selected from dermatomyositis, polymyositis, inclusion body myositis, and juvenile myositis, or another autoimmune disorder (e.g., myasthenia gravis);
(iv) the subject has or is at risk of developing muscle wasting due to an infection, which optionally is selected from an infection by a virus (e.g., HIV, influenza virus, and Epstein-Barr virus), a bacterium (e.g., pyomyositis, S. aureus, and streptococci), a spirochete (e.g., Lyme disease), and a parasite (e.g., trichinosis);
(v) the subject has or is at risk of developing muscle wasting due to exposure to a toxin, which optionally is selected from a medication (e.g., a cholesterol-lowering medication (e.g., a statin), propofol, amiodaron, colchicine, chloroquine, an antiviral, a protease inhibitor, omeprazole, and tryptophan), alcohol, and toluene;
(vi) the subject has or is at risk of developing muscle wasting due to a systemic disease, which optionally is selected from an endocrine disorder (e.g., thyroid, parathyroid, pituitary, and adrenal disorder), systemic inflammatory disease (e.g., systemic lupus erythematosus, rheumatoid arthritis, scleroderma, Sjogren's syndrome, mixed connective disease, and sarcoidosis), electrolyte imbalance, critical illness myopathy, and amyloid myopathy;
(vii) the subject has or is at risk of developing muscle wasting due to a neurological disease or condition, which optionally is selected from amyotrophic lateral sclerosis (ALS), multiple sclerosis (MS), spinal muscular atrophy (SMA), Guillain-Barre syndrome, carpel tunnel syndrome, Charcot-Marie-Tooth disease, Parkinson's disease, Lewy body disease, polio, spinal cord injury, peripheral nerve injury, stroke, and nerve damage caused by diabetes, toxins, or alcohol;
(viii) the subject has or is at risk of developing muscle wasting due to presence in an environment with decreased or no gravity, e.g., during a space flight or being at a space station or the like;
(ix) the subject has or is at risk of developing muscle wasting due to prolonged inactivity (e.g., is bedridden, has a seated job or a sedentary lifestyle, or cannot move limbs due to stroke or other brain disease), burn, long-term corticosteroid therapy, malnutrition, anorexia, cachexia, osteoarthritis, advanced age, cancer, chronic kidney disease, heart failure, or chronic obstructive pulmonary disease; or
(x) the subject has or is at risk of developing one or more symptom selected from muscle loss, movement issues, balance problems, trouble walking, trouble using arms or legs, contractures, muscle rigidity, muscle twitching, fasciculations, muscle cramps, numbness, tingling, painful sensations, breathing problems, curved spine, heart problems, swallowing problems (which may lead to, e.g., nutritional problems and aspiration pneumonia), reduced muscle mass (e.g., in general or in one limb), weakness (e.g., in general or in one limb), pain, discomfort, one arm or leg being smaller than the other, weakness in one arm or leg, numbness or tingling in an arm or leg, trouble walking, balance problems, facia weakness, gradual memory loss, reduced muscle strength, impaired ability to perform physical activities, decrease in muscle size, cramps, stiffness, spasms, fatigue, motor delay, respiratory impairment, and bulbar muscle dysfunction.

68-77. (canceled)

78. A method of maintaining or improving muscle physiology in a subject, the method comprising administering to the subject an effective amount of an immunotherapy targeting myostatin and/or activin A, wherein the immunotherapy comprises a peptide immunogen construct of claim 37; optionally wherein the maintaining or improving muscle physiology is maintaining or increasing muscle mass, muscle strength, muscle tone, and/or muscle endurance.

79. (canceled)

80. A method of treating or preventing muscular or musculoskeletal injury in a subject, the method comprising administering to the subject an effective amount of an immunotherapy targeting myostatin and/or activin A, wherein the immunotherapy comprises a peptide immunogen construct of claim 37; optionally wherein the muscular or musculoskeletal injury is muscle tear or muscle strain.

81. (canceled)

82. A method of treating or preventing a disorder associated with muscle loss or insufficient muscle growth in a subject, the method comprising administering to the subject an effective amount of an immunotherapy targeting myostatin and/or activin A, wherein the immunotherapy comprises a peptide immunogen construct of claim 37; optionally wherein the subject has muscle atrophy; further optionally wherein the disorder is cachexia or muscle loss associated with a cancer or tumor.

83-84. (canceled)

85. A method of treating or preventing sarcopenia or a symptom thereof in a subject (optionally age-related sarcopenia), the method comprising administering to the subject an effective amount of an immunotherapy targeting myostatin and/or activin A, wherein the immunotherapy comprises a peptide immunogen construct of claim 37.

86. The method of claim 63, wherein the subject is a human, optionally wherein the subject is at least or over 55 years old, 60 years old, 65 years old, 70 years old, 75 years old, or 80 years old.

87. (canceled)

88. A pharmaceutical composition or a kit comprising:
(i) a peptide immunogen construct comprising a myostatin B cell epitope, a heterologous T cell (Th) epitope, and an optional heterologous spacer, wherein the B cell epitope is covalently linked to the Th epitope directly or through the optional heterologous spacer; wherein the myostatin B cell epitope comprises about 7 to about 30 contiguous amino acids of SEQ ID NO: 1 or SEQ ID NO: 2 or a variant thereof; and
(ii) a peptide immunogen construct comprising an activin A B cell epitope, a heterologous T cell (Th) epitope, and an optional heterologous spacer, wherein the B cell epitope is covalently linked to the Th epitope directly or through the optional heterologous spacer, wherein the activin A B cell epitope comprises about 7 to about 30 contiguous amino acids of SEQ ID NO: 12 or a variant thereof.

89-90. (canceled)


SEQUENCE LISTING (193 sequences)

SEQ ID NO: 1 - protein, 375 aa, synthetic construct
  REGION 1..375: note = Synthetic
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 3 - protein, 11 aa, synthetic construct
  REGION 1..11: note = Synthetic
CDEHSTESRSC

SEQ ID NO: 4 - protein, 23 aa, synthetic construct
  REGION 1..23: note = Synthetic
CLTVDFEAFGWDWIIAPKRYKAC

SEQ ID NO: 5 - protein, 20 aa, synthetic construct
  REGION 1..20: note = Synthetic
CKYPHTHLVHQANPRGSAGC

SEQ ID NO: 6 - protein, 19 aa, synthetic construct
  REGION 1..19: note = Synthetic
CINMLYFNGKEQIIYGKIC

SEQ ID NO: 7 - protein, 25 aa, synthetic construct
  REGION 1..25: note = Synthetic
CFVFLQKYPHTHLVHQANPRGSAGC

SEQ ID NO: 8 - protein, 26 aa, synthetic construct
  REGION 1..26: note = Synthetic
CEFVFLQKYPHTHLVHQANPRGSAGC

SEQ ID NO: 9 - protein, 28 aa, synthetic construct
  REGION 1..28: note = Synthetic
CESEFVFLQKYPHTHLVHQANPRGSAGC

SEQ ID NO: 10 - protein, 12 aa, synthetic construct
  REGION 1..12: note = Synthetic
CSGESEFVFLQC

SEQ ID NO: 11 - protein, 426 aa, synthetic construct
  REGION 1..426: note = Synthetic
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 12 - protein, 116 aa, synthetic construct [sequence omitted]

SEQ ID NO: 13 - protein, 9 aa, synthetic construct
  REGION 1..9: note = Synthetic
CDGKVNISC

SEQ ID NO: 14 - protein, 16 aa, synthetic construct
  REGION 1..16: note = Synthetic
CFKDIGWNDWIIAPSC

SEQ ID NO: 15 - protein, 27 aa, synthetic construct
  REGION 1..27: note = Synthetic
CSLSFHSTVINHYRMRGHSPFANLKSC

SEQ ID NO: 16 - protein, 17 aa, synthetic construct
  REGION 1..17: note = Synthetic
CPSHIAGTSGSSLSFHC

SEQ ID NO: 17 - protein, 21 aa, synthetic construct
  REGION 1..21: note = Synthetic
CTVINHYRMRGHSPFANLKSC

SEQ ID NO: 18 - protein, 17 aa, synthetic construct
  REGION 1..17: note = Synthetic
CSMLYYDDGQNIIKKDC

SEQ ID NO: 19 - protein, 17 aa, synthetic construct
  REGION 1..17: note = Synthetic
KKQYIKANSKFIGITEL

SEQ ID NO: 20 - protein, 15 aa, synthetic construct
  REGION 1..15: note = Synthetic
LSEIKGVIVHRLEGV

SEQ ID NO: 21 - protein, 24 aa, synthetic construct
  REGION 1..24: note = Synthetic
GAYARCPNGTRALTVAELRGNAEL

SEQ ID NO: 22 - protein, 17 aa, synthetic construct
  REGION 1..17: note = Synthetic
WVRDIIDDFTNESSQKT

SEQ ID NO: 23 - protein, 23 aa, synthetic construct
  REGION 1..23: note = Synthetic
DSETADNLEKTVAALSILPGHGC

SEQ ID NO: 24 - protein, 21 aa, synthetic construct
  REGION 1..21: note = Synthetic
DHEKKHAKMEKASSVFNVVNS

SEQ ID NO: 25 - protein, 17 aa, synthetic construct
  REGION 1..17: note = Synthetic
KWFKTNAPNGVDEKHRH

SEQ ID NO: 26 - protein, 25 aa, synthetic construct
  REGION 1..25: note = Synthetic
ALNIWDRFDVFCTLGATTGYLKGNS

SEQ ID NO: 27 - protein, 15 aa, synthetic construct
  REGION 1..15: note = Synthetic
ISEIKGVIVHKIEGI

SEQ ID NO: 28 - protein, 22 aa, synthetic construct
  REGION 1..22: note = Synthetic
  VARIANT 7: note = can be T
  VARIANT 10: note = can be R
  VARIANT 11: note = can be T
  VARIANT 15: note = can be T
  VARIANT 16: note = can be R
  VARIANT 19: note = can be T
KKKISISEIKGVIVHKIEGILF

SEQ ID NO: 29 - protein, 18 aa, synthetic construct
  REGION 1..18: note = Synthetic
  VARIANT 2: note = can be R
  VARIANT 3: note = can be R
  VARIANT 4: note = can be I, V or F
  VARIANT 5: note = can be K, R
  VARIANT 6: note = can be I, V or F
  VARIANT 7: note = can be I, V or F
  VARIANT 9: note = can be R
  VARIANT 10: note = can be I, V or F
  VARIANT 11: note = can be I, V or F
  VARIANT 13: note = can be I, V or F
  VARIANT 15: note = can be L, I, V or F
  VARIANT 17: note = can be I, V or F
  VARIANT 18: note = can be R
KKKLFLLTKLLTLPQSLD

SEQ ID NO: 30 - protein, 19 aa, synthetic construct
  REGION 1..19: note = Synthetic
  VARIANT 4: note = can be T
  VARIANT 7: note = can be R
  VARIANT 8: note = can be T
  VARIANT 12: note = can be T
  VARIANT 13: note = can be R
ISISEIKGVIVHKIETILF

SEQ ID NO: 31 - protein, 18 aa, synthetic construct
  REGION 1..18: note = Synthetic
  VARIANT 4: note = can be F
  VARIANT 5: note = can be F
  VARIANT 6: note = can be L
  VARIANT 7: note = can be L
  VARIANT 11: note = can be L
  VARIANT 14: note = can be I
  VARIANT 15: note = can be T
  VARIANT 16: note = can be T
  VARIANT 17: note = can be I
KKKIITITRIITIPQSLD

SEQ ID NO: 32 - protein, 19 aa, synthetic construct
  REGION 1..19: note = Synthetic
ISITEIKGVIVHRIETILF

SEQ ID NO: 33 - protein, 18 aa, synthetic construct
  REGION 1..18: note = Synthetic
KKKIITITRIITIITTID

SEQ ID NO: 34 - protein, 11 aa, synthetic construct
  REGION 1..11: note = Synthetic
FVFTLTVPSER

SEQ ID NO: 35 - protein, 15 aa, synthetic construct
  REGION 1..15: note = Synthetic
SGPLKAEIAQRLEDV

SEQ ID NO: 36 - protein, 9 aa, synthetic construct
  REGION 1..9: note = Synthetic
DRLRRDQKS

SEQ ID NO: 37 - protein, 19 aa, synthetic construct
  REGION 1..19: note = Synthetic
AGLTLSLLVICSYLFISRG

SEQ ID NO: 38 - protein, 15 aa, synthetic construct
  REGION 1..15: note = Synthetic
QYIKANSKFIGITEL

SEQ ID NO: 39 - protein, 20 aa, synthetic construct
  REGION 1..20: note = Synthetic
PGPLRESIVCYFMVFLQTHI

SEQ ID NO: 40 - protein, 21 aa, synthetic construct
  REGION 1..21: note = Synthetic
FNNFTVSFWLRVPKVSASHLE

SEQ ID NO: 41 - protein, 16 aa, synthetic construct
  REGION 1..16: note = Synthetic
KFIIKRYTPNNEIDSF

SEQ ID NO: 42 - protein, 16 aa, synthetic construct
  REGION 1..16: note = Synthetic
VSIDKFRIFCKALNPK

SEQ ID NO: 43 - protein, 18 aa, synthetic construct
  REGION 1..18: note = Synthetic
VPGLYSPCRAFFNKEELL

SEQ ID NO: 44 - protein, 14 aa, synthetic construct
  REGION 1..14: note = Synthetic
DKREMWMACIKELH

SEQ ID NO: 45 - protein, 15 aa, synthetic construct
  REGION 1..15: note = Synthetic
TGHGARTSTEPTTDY

SEQ ID NO: 46 - protein, 13 aa, synthetic construct
  REGION 1..13: note = Synthetic
KELKRQYEKKLRQ

SEQ ID NO: 47 - protein, 11 aa, synthetic construct
  REGION 1..11: note = Synthetic
TVFYNIPPMPL

SEQ ID NO: 48 - protein, 31 aa, synthetic construct
  REGION 1..31: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKCDEHSTESRSC

SEQ ID NO: 49 - protein, 31 aa, synthetic construct
  REGION 1..31: note = Synthetic
  MOD_RES 12: note = epsilon lysine
  VARIANT 16: note = can be T
  VARIANT 19: note = can be R
  VARIANT 20: note = can be T
  VARIANT 24: note = can be T
  VARIANT 25: note = can be R
CDEHSTESRSCKISISEIKGVIVHKIETILF

SEQ ID NO: 50 - protein, 51 aa, synthetic construct
  REGION 1..51: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 32: note = epsilon lysine
  VARIANT 36: note = can be T
  VARIANT 39: note = can be R
  VARIANT 40: note = can be T
  VARIANT 44: note = can be T
  VARIANT 45: note = can be R
ISISEIKGVIVHKIETILFKCDEHSTESRSCKISISEIKGVIVHKIETILF

SEQ ID NO: 51 - protein, 46 aa, synthetic construct
  REGION 1..46: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKKKKCLTVDFEAFGWDWIIAPKRYKAC

SEQ ID NO: 52 - protein, 46 aa, synthetic construct
  REGION 1..46: note = Synthetic
  MOD_RES 27: note = epsilon lysine
  VARIANT 31: note = can be T
  VARIANT 34: note = can be R
  VARIANT 35: note = can be T
  VARIANT 39: note = can be T
  VARIANT 40: note = can be R
CLTVDFEAFGWDWIIAPKRYKACKKKKISISEIKGVIVHKIETILF

SEQ ID NO: 53 - protein, 69 aa, synthetic construct
  REGION 1..69: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 50: note = epsilon lysine
  VARIANT 54: note = can be T
  VARIANT 57: note = can be R
  VARIANT 58: note = can be T
  VARIANT 62: note = can be T
  VARIANT 63: note = can be R
ISISEIKGVIVHKIETILFKKKKCLTVDFEAFGWDWIIAPKRYKACKKKKISISEIKGVIVHKIETILF

SEQ ID NO: 54 - protein, 43 aa, synthetic construct
  REGION 1..43: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKKKKCKYPHTHLVHQANPRGSAGC

SEQ ID NO: 55 - protein, 43 aa, synthetic construct
  REGION 1..43: note = Synthetic
  MOD_RES 24: note = epsilon lysine
  VARIANT 28: note = can be T
  VARIANT 31: note = can be R
  VARIANT 32: note = can be T
  VARIANT 36: note = can be T
  VARIANT 37: note = can be R
CKYPHTHLVHQANPRGSAGCKKKKISISEIKGVIVHKIETILF

SEQ ID NO: 56 - protein, 66 aa, synthetic construct
  REGION 1..66: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 47: note = epsilon lysine
  VARIANT 51: note = can be T
  VARIANT 54: note = can be R
  VARIANT 55: note = can be T
  VARIANT 59: note = can be T
  VARIANT 60: note = can be R
ISISEIKGVIVHKIETILFKKKKCKYPHTHLVHQANPRGSAGCKKKKISISEIKGVIVHKIETILF

SEQ ID NO: 57 - protein, 42 aa, synthetic construct
  REGION 1..42: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKKKKCINMLYFNGKEQIIYGKIC

SEQ ID NO: 58 - protein, 42 aa, synthetic construct
  REGION 1..42: note = Synthetic
  MOD_RES 23: note = epsilon lysine
  VARIANT 27: note = can be T
  VARIANT 30: note = can be R
  VARIANT 31: note = can be T
  VARIANT 35: note = can be T
  VARIANT 36: note = can be R
CINMLYFNGKEQIIYGKICKKKKISISEIKGVIVHKIETILF

SEQ ID NO: 59 - protein, 65 aa, synthetic construct
  REGION 1..65: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 46: note = epsilon lysine
  VARIANT 50: note = can be T
  VARIANT 53: note = can be R
  VARIANT 54: note = can be T
  VARIANT 58: note = can be T
  VARIANT 59: note = can be R
ISISEIKGVIVHKIETILFKKKKCINMLYFNGKEQIIYGKICKKKKISISEIKGVIVHKIETILF

SEQ ID NO: 60 - protein, 48 aa, synthetic construct
  REGION 1..48: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKKKKCFVFLQKYPHTHLVHQANPRGSAGC

SEQ ID NO: 61 - protein, 48 aa, synthetic construct
  REGION 1..48: note = Synthetic
  MOD_RES 29: note = epsilon lysine
  VARIANT 33: note = can be T
  VARIANT 36: note = can be R
  VARIANT 37: note = can be T
  VARIANT 41: note = can be T
  VARIANT 42: note = can be R
CFVFLQKYPHTHLVHQANPRGSAGCKKKKISISEIKGVIVHKIETILF

SEQ ID NO: 62 - protein, 71 aa, synthetic construct
  REGION 1..71: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 52: note = epsilon lysine
  VARIANT 56: note = can be T
  VARIANT 59: note = can be R
  VARIANT 60: note = can be T
  VARIANT 64: note = can be T
  VARIANT 65: note = can be R
ISISEIKGVIVHKIETILFKKKKCFVFLQKYPHTHLVHQANPRGSAGCKKKKISISEIKGVIVHKIETILF

SEQ ID NO: 63 - protein, 49 aa, synthetic construct
  REGION 1..49: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKKKKCEFVFLQKYPHTHLVHQANPRGSAGC

SEQ ID NO: 64 - protein, 49 aa, synthetic construct
  REGION 1..49: note = Synthetic
  MOD_RES 30: note = epsilon lysine
  VARIANT 34: note = can be T
  VARIANT 37: note = can be R
  VARIANT 38: note = can be T
  VARIANT 42: note = can be T
  VARIANT 43: note = can be R
CEFVFLQKYPHTHLVHQANPRGSAGCKKKKISISEIKGVIVHKIETILF

SEQ ID NO: 65 - protein, 72 aa, synthetic construct
  REGION 1..72: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 53: note = epsilon lysine
  VARIANT 57: note = can be T
  VARIANT 60: note = can be R
  VARIANT 61: note = can be T
  VARIANT 65: note = can be T
  VARIANT 66: note = can be R
ISISEIKGVIVHKIETILFKKKKCEFVFLQKYPHTHLVHQANPRGSAGCKKKKISISEIKGVIVHKIETILF

SEQ ID NO: 66 - protein, 51 aa, synthetic construct
  REGION 1..51: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKKKKCESEFVFLQKYPHTHLVHQANPRGSAGC

SEQ ID NO: 67 - protein, 51 aa, synthetic construct
  REGION 1..51: note = Synthetic
  MOD_RES 32: note = epsilon lysine
  VARIANT 36: note = can be T
  VARIANT 39: note = can be R
  VARIANT 40: note = can be T
  VARIANT 44: note = can be T
  VARIANT 45: note = can be R
CESEFVFLQKYPHTHLVHQANPRGSAGCKKKKISISEIKGVIVHKIETILF

SEQ ID NO: 68 - protein, 74 aa, synthetic construct
  REGION 1..74: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 55: note = epsilon lysine
  VARIANT 59: note = can be T
  VARIANT 62: note = can be R
  VARIANT 63: note = can be T
  VARIANT 67: note = can be T
  VARIANT 68: note = can be R
ISISEIKGVIVHKIETILFKKKKCESEFVFLQKYPHTHLVHQANPRGSAGCKKKKISISEIKGVIVHKIETILF

SEQ ID NO: 69 - protein, 35 aa, synthetic construct
  REGION 1..35: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKKKKCSGESEFVFLQC

SEQ ID NO: 70 - protein, 35 aa, synthetic construct
  REGION 1..35: note = Synthetic
  MOD_RES 16: note = epsilon lysine
  VARIANT 20: note = can be T
  VARIANT 23: note = can be R
  VARIANT 24: note = can be T
  VARIANT 28: note = can be T
  VARIANT 29: note = can be R
CSGESEFVFLQCKKKKISISEIKGVIVHKIETILF

SEQ ID NO: 71 - protein, 58 aa, synthetic construct
  REGION 1..58: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 39: note = epsilon lysine
  VARIANT 43: note = can be T
  VARIANT 46: note = can be R
  VARIANT 47: note = can be T
  VARIANT 51: note = can be T
  VARIANT 52: note = can be R
ISISEIKGVIVHKIETILFKKKKCSGESEFVFLQCKKKKISISEIKGVIVHKIETILF

SEQ ID NO: 72 - protein, 29 aa, synthetic construct
  REGION 1..29: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKCDGKVNISC

SEQ ID NO: 73 - protein, 29 aa, synthetic construct
  REGION 1..29: note = Synthetic
  MOD_RES 1..29: note = epsilon lysine
  VARIANT 14: note = can be T
  VARIANT 17: note = can be R
  VARIANT 18: note = can be T
  VARIANT 22: note = can be T
  VARIANT 23: note = can be R
CDGKVNISCKISISEIKGVIVHKIETILF

SEQ ID NO: 74 - protein, 49 aa, synthetic construct
  REGION 1..49: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 30: note = epsilon lysine
  VARIANT 34: note = can be T
  VARIANT 37: note = can be R
  VARIANT 38: note = can be T
  VARIANT 42: note = can be T
  VARIANT 43: note = can be R
ISISEIKGVIVHKIETILFKCDGKVNISCKISISEIKGVIVHKIETILF

SEQ ID NO: 75 - protein, 36 aa, synthetic construct
  REGION 1..36: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKCFKDIGWNDWIIAPSC

SEQ ID NO: 76 - protein, 36 aa, synthetic construct
  REGION 1..36: note = Synthetic
  MOD_RES 17: note = epsilon lysine
  VARIANT 21: note = can be T
  VARIANT 24: note = can be R
  VARIANT 25: note = can be T
  VARIANT 29: note = can be T
  VARIANT 30: note = can be R
CFKDIGWNDWIIAPSCKISISEIKGVIVHKIETILF

SEQ ID NO: 77 - protein, 56 aa, synthetic construct
  REGION 1..56: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 37: note = epsilon lysine
  VARIANT 41: note = can be T
  VARIANT 44: note = can be R
  VARIANT 45: note = can be T
  VARIANT 49: note = can be T
  VARIANT 50: note = can be R
ISISEIKGVIVHKIETILFKCFKDIGWNDWIIAPSCKISISEIKGVIVHKIETILF

SEQ ID NO: 78 - protein, 47 aa, synthetic construct
  REGION 1..47: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKCSLSFHSTVINHYRMRGHSPFANLKSC

SEQ ID NO: 79 - protein, 47 aa, synthetic construct
  REGION 1..47: note = Synthetic
  MOD_RES 28: note = epsilon lysine
  VARIANT 32: note = can be T
  VARIANT 35: note = can be R
  VARIANT 36: note = can be T
  VARIANT 40: note = can be T
  VARIANT 41: note = can be R
CSLSFHSTVINHYRMRGHSPFANLKSCKISISEIKGVIVHKIETILF

SEQ ID NO: 80 - protein, 67 aa, synthetic construct
  REGION 1..67: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 48: note = epsilon lysine
  VARIANT 52: note = can be T
  VARIANT 55: note = can be R
  VARIANT 56: note = can be T
  VARIANT 60: note = can be T
  VARIANT 61: note = can be R
ISISEIKGVIVHKIETILFKCSLSFHSTVINHYRMRGHSPFANLKSCKISISEIKGVIVHKIETILF

SEQ ID NO: 81 - protein, 37 aa, synthetic construct
  REGION 1..37: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKCPSHIAGTSGSSLSFHC

SEQ ID NO: 82 - protein, 37 aa, synthetic construct
  REGION 1..37: note = Synthetic
  MOD_RES 18: note = epsilon lysine
  VARIANT 22: note = can be T
  VARIANT 25: note = can be R
  VARIANT 26: note = can be T
  VARIANT 30: note = can be T
  VARIANT 31: note = can be R
CPSHIAGTSGSSLSFHCKISISEIKGVIVHKIETILF

SEQ ID NO: 83 - protein, 57 aa, synthetic construct
  REGION 1..57: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 38: note = epsilon lysine
  VARIANT 42: note = can be T
  VARIANT 45: note = can be R
  VARIANT 46: note = can be T
  VARIANT 50: note = can be T
  VARIANT 51: note = can be R
ISISEIKGVIVHKIETILFKCPSHIAGTSGSSLSFHCKISISEIKGVIVHKIETILF

SEQ ID NO: 84 - protein, 41 aa, synthetic construct
  REGION 1..41: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKCTVINHYRMRGHSPFANLKSC

SEQ ID NO: 85 - protein, 41 aa, synthetic construct
  REGION 1..41: note = Synthetic
  MOD_RES 22: note = epsilon lysine
  VARIANT 26: note = can be T
  VARIANT 29: note = can be R
  VARIANT 30: note = can be T
  VARIANT 34: note = can be T
  VARIANT 35: note = can be R
CTVINHYRMRGHSPFANLKSCKISISEIKGVIVHKIETILF

SEQ ID NO: 86 - protein, 61 aa, synthetic construct
  REGION 1..61: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 42: note = epsilon lysine
  VARIANT 46: note = can be T
  VARIANT 49: note = can be R
  VARIANT 50: note = can be T
  VARIANT 54: note = can be T
  VARIANT 55: note = can be R
ISISEIKGVIVHKIETILFKCTVINHYRMRGHSPFANLKSCKISISEIKGVIVHKIETILF

SEQ ID NO: 87 - protein, 37 aa, synthetic construct
  REGION 1..37: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 20: note = epsilon lysine
ISISEIKGVIVHKIETILFKCSMLYYDDGQNIIKKDC

SEQ ID NO: 88 - protein, 37 aa, synthetic construct
  REGION 1..37: note = Synthetic
  MOD_RES 18: note = epsilon lysine
  VARIANT 22: note = can be T
  VARIANT 25: note = can be R
  VARIANT 26: note = can be T
  VARIANT 30: note = can be T
  VARIANT 31: note = can be R
CSMLYYDDGQNIIKKDCKISISEIKGVIVHKIETILF

SEQ ID NO: 89 - protein, 57 aa, synthetic construct
  REGION 1..57: note = Synthetic
  VARIANT 4: note = can be T
  VARIANT 7: note = can be R
  VARIANT 8: note = can be T
  VARIANT 12: note = can be T
  VARIANT 13: note = can be R
  MOD_RES 38: note = epsilon lysine
  VARIANT 42: note = can be T
  VARIANT 45: note = can be R
  VARIANT 46: note = can be T
  VARIANT 50: note = can be T
  VARIANT 51: note = can be R
ISISEIKGVIVHKIETILFKCSMLYYDDGQNIIKKDCKISISEIKGVIVHKIETILF

SEQ ID NO: 90 - protein, 6 aa, synthetic construct
  REGION 1..6: note = Synthetic
  VARIANT 3: note = X is any amino acid or is aspartic acid
  VARIANT 5: note = X is any amino acid or is aspartic acid
PPXPXP

SEQ ID NO: 91 - protein, 4 aa, synthetic construct
  REGION 1..4: note = Synthetic
  MOD_RES 1: note = epsilon N-terminus lysine
KKKK

SEQ ID NO: 92 - DNA, 32 bp, synthetic construct
  misc_feature 1..32: note = Synthetic
  source 1..32: mol_type = other DNA
tcgtcgttttgtcgttttgtcgttttgtcgtt

SEQ ID NO: 93 - DNA, 24 bp, synthetic construct
  misc_feature 1..24: note = Synthetic
  source 1..24: mol_type = other DNA
tcgtcgttttgtcgttttgtcgtt

SEQ ID NO: 94 - DNA, 24 bp, synthetic construct
  misc_feature 1..24: note = Synthetic
  source 1..24: mol_type = other DNA
tcgtcgttttgtcgttttgtcgtt

SEQ ID NO: 95 - protein, 10 aa, synthetic construct
  REGION 1..10: note = Synthetic
EFVFLQKYPH

SEQ ID NO: 96 - protein, 10 aa, synthetic construct
  REGION 1..10: note = Synthetic
ECEFVFCQKY

SEQ ID NO: 97 - protein, 20 aa, synthetic construct
  REGION 1..20: note = Synthetic
QKYPHTHLVHQANPRGSAGP

SEQ ID NO: 98 - protein, 20 aa, synthetic construct
  REGION 1..20: note = Synthetic
CKYPHTHLVHQANPRGSAGC

SEQ ID NO: 99 - protein, 17 aa, synthetic construct
  REGION 1..17: note = Synthetic
ECEFVFCQKYPHTHLVH

SEQ ID NO: 100 - protein, 25 aa, synthetic construct
  REGION 1..25: note = Synthetic
EFVFLQKYPHTHLVHQANPRGSAGP

SEQ ID NO: 101 - protein, 27 aa, synthetic construct
  REGION 1..27: note = Synthetic
CEFVFLQKYPHTHLVHQANPRGSAGPC

SEQ ID NO: 102 - protein, 17 aa, synthetic construct
  REGION 1..17: note = Synthetic
EFVFLQKYPHTHLVHQA

SEQ ID NO: 103 - protein, 20 aa, synthetic construct
  MOD_RES 1: note = Ahx (6-aminohexanoic acid)
XKYPHTHLVHQANPRGSAGE

SEQ ID NO: 104 - protein, 18 aa, synthetic construct
  REGION 1..18: note = Synthetic
QKYPCTHLVHQANPRGSC

SEQ ID NO: 105 - protein, 18 aa, synthetic construct
  REGION 1..18: note = Synthetic
  MOD_RES 18: note = Dap (L-2,3 diaminopropionic acid)
QKYPDTHLVHQANPRGSX

SEQ ID NO: 106 - protein, 16 aa, synthetic construct
  REGION 1..16: note = Synthetic
PHTHLVHQANPRGSAE

SEQ ID NO: 107 - protein, 8 aa, synthetic construct
  REGION 1..8: note = Synthetic
EFVFLQKY

SEQ ID NO: 108 - protein, 23 aa, synthetic construct
  REGION 1..23: note = Synthetic
EFVFLQKYPCTHLVHQANPRGSC

SEQ ID NO: 109 - protein, 20 aa, synthetic construct
  REGION 1..20: note = Synthetic
  MOD_RES 20: note = Dap (L-2,3 diaminopropionic acid)
DKYPHTHLVHQANPRGSAGX

SEQ ID NO: 110 - protein, 11 aa, synthetic construct
  REGION 1..11: note = Synthetic
DIGCNDWIIAC

SEQ ID NO: 111 - protein, 10 aa, synthetic construct
  REGION 1..10: note = Synthetic
WNDWIIAPSG

SEQ ID NO: 112 - protein, 10 aa, synthetic construct
  REGION 1..10: note = Synthetic
STVINHYRMR

SEQ ID NO: 113 - protein, 10 aa, synthetic construct
  REGION 1..10: note = Synthetic
HYRMRGHSPF

SEQ ID NO: 114 - protein, 10 aa, synthetic construct
  REGION 1..10: note = Synthetic
HSPFANLKSS

SEQ ID NO: 115 - protein, 9 aa, synthetic construct
  REGION 1..9: note = Synthetic
FKDIGWNDW

SEQ ID NO: 116 - protein, 21 aa, synthetic construct
  REGION 1..21: note = Synthetic
STVINHYRMRGHSPFANLKSS

SEQ ID NO: 117 - protein, 21 aa, synthetic construct
  REGION 1..21: note = Synthetic
CTVINHYRMRGHSPFANLKSC

SEQ ID NO: 118 - protein, 16 aa, synthetic construct
  REGION 1..16: note = Synthetic
CFKDIGWNDWIIAPSC

SEQ ID NO: 119 - protein, 16 aa, synthetic construct
  REGION 1..16: note = Synthetic
SFKDIGWNDWIIAPSG

SEQ ID NO: 120 - protein, 33 aa, synthetic construct
  REGION 1..33: note = Synthetic
  MOD_RES 14: note = epsilon lysine
EFVFLQKYPHKKKKISITEIKGVIVHRIETILF

SEQ ID NO: 121 - protein, 33 aa, synthetic construct
  REGION 1..33: note = Synthetic
  MOD_RES 14: note = epsilon lysine
ECEFVFCQKYKKKKISITEIKGVIVHRIETILF

SEQ ID NO: 122 - protein, 43 aa, synthetic construct
  REGION 1..43: note = Synthetic
  MOD_RES 24: note = epsilon lysine
QKYPHTHLVHQANPRGSAGPKKKKISITEIKGVIVHRIETILF

SEQ ID NO: 123 - protein, 43 aa, synthetic construct
  REGION 1..43: note = Synthetic
  MOD_RES 24: note = epsilon lysine
CKYPHTHLVHQANPRGSAGCKKKKISITEIKGVIVHRIETILF

SEQ ID NO: 124 - protein, 40 aa, synthetic construct
  REGION 1..40: note = Synthetic
  MOD_RES 21: note = epsilon lysine
ECEFVFCQKYPHTHLVHKKKKISITEIKGVIVHRIETILF

SEQ ID NO: 125 - protein, 48 aa, synthetic construct
  REGION 1..48: note = Synthetic
  MOD_RES 29: note = epsilon lysine
EFVFLQKYPHTHLVHQANPRGSAGPKKKKISITEIKGVIVHRIETILF

SEQ ID NO: 126 - protein, 50 aa, synthetic construct
  REGION 1..50: note = Synthetic
  MOD_RES 31: note = epsilon lysine
CEFVFLQKYPHTHLVHQANPRGSAGPCKKKKISITEIKGVIVHRIETILF

SEQ ID NO: 127 - protein, 40 aa, synthetic construct
  REGION 1..40: note = Synthetic
  MOD_RES 21: note = epsilon lysine
EFVFLQKYPHTHLVHQAKKKKISITEIKGVIVHRIETILF

SEQ ID NO: 128 - protein, 43 aa, synthetic construct
  MOD_RES 24: note = epsilon lysine
  MOD_RES 1: note = Ahx (6-aminohexanoic acid)
XKYPHTHLVHQANPRGSAGEKKKKISITEIKGVIVHRIETILF

SEQ ID NO: 129 - protein, 41 aa, synthetic construct
  REGION 1..41: note = Synthetic
  MOD_RES 22: note = epsilon lysine
QKYPCTHLVHQANPRGSCKKKKISITEIKGVIVHRIETILF

SEQ ID NO: 130 - protein, 41 aa, synthetic construct
  REGION 1..41: note = Synthetic
  MOD_RES 22: note = epsilon lysine
  MOD_RES 18: note = Dap (L-2,3 diaminopropionic acid)
QKYPDTHLVHQANPRGSXKKKKISITEIKGVIVHRIETILF

SEQ ID NO: 131 - protein, 39 aa, synthetic construct
  REGION 1..39: note = Synthetic
  MOD_RES 20: note = epsilon lysine
PHTHLVHQANPRGSAEKKKKISITEIKGVIVHRIETILF

SEQ ID NO: 132 - protein, 31 aa, synthetic construct
  REGION 1..31: note = Synthetic
  MOD_RES 12: note = epsilon lysine
EFVFLQKYKKKKISITEIKGVIVHRIETILF

SEQ ID NO: 133 - protein, 46 aa, synthetic construct
  REGION 1..46: note = Synthetic
  MOD_RES 27: note = epsilon lysine
EFVFLQKYPCTHLVHQANPRGSCKKKKISITEIKGVIVHRIETILF

SEQ ID NO: 134 - protein, 43 aa, synthetic construct
  REGION 1..43: note = Synthetic
  MOD_RES 24: note = epsilon lysine
  MOD_RES 20: note = (Dap) L-2,3 diaminopropionic acid
DKYPHTHLVHQANPRGSAGXKKKKISITEIKGVIVHRIETILF

SEQ ID NO: 135 - protein, 34 aa, synthetic construct
  REGION 1..34: note = Synthetic
  MOD_RES 15: note = epsilon lysine
DIGCNDWIIACKKKKISITEIKGVIVHRIETILF

SEQ ID NO: 136 - protein, 33 aa, synthetic construct
  REGION 1..33: note = Synthetic
  MOD_RES 14: note = epsilon lysine
WNDWIIAPSGKKKKISITEIKGVIVHRIETILF

SEQ ID NO: 137 - protein, 33 aa, synthetic construct
  REGION 1..33: note = Synthetic
  MOD_RES 14: note = epsilon lysine
STVINHYRMRKKKKISITEIKGVIVHRIETILF

SEQ ID NO: 138 - protein, 33 aa, synthetic construct
  REGION 1..33: note = Synthetic
  MOD_RES 14: note = epsilon lysine
HYRMRGHSPFKKKKISITEIKGVIVHRIETILF

SEQ ID NO: 139 - protein, 33 aa, synthetic construct
  REGION 1..33: note = Synthetic
  MOD_RES 14: note = epsilon lysine
HSPFANLKSSKKKKISITEIKGVIVHRIETILF

SEQ ID NO: 140 - protein, 32 aa, synthetic construct
  REGION 1..32: note = Synthetic
  MOD_RES 13: note = epsilon lysine
FKDIGWNDWKKKKISITEIKGVIVHRIETILF

SEQ ID NO: 141 - protein, 44 aa, synthetic construct
  REGION 1..44: note = Synthetic
  MOD_RES 25: note = epsilon lysine
STVINHYRMRGHSPFANLKSSKKKKISITEIKGVIVHRIETILF

SEQ ID NO: 142 - protein, 44 aa, synthetic construct
  REGION 1..44: note = Synthetic
  MOD_RES 25: note = epsilon lysine
CTVINHYRMRGHSPFANLKSCKKKKISITEIKGVIVHRIETILF

SEQ ID NO: 143 - protein, 39 aa, synthetic construct
  REGION 1..39: note = Synthetic
  MOD_RES 20: note = epsilon lysine
CFKDIGWNDWIIAPSCKKKKISITEIKGVIVHRIETILF

SEQ ID NO: 144 - protein, 39 aa, synthetic construct
  REGION 1..39: note = Synthetic
  MOD_RES 20: note = epsilon lysine
SFKDIGWNDWIIAPSGKKKKISITEIKGVIVHRIETILF

SEQ ID NO: 145 - protein, 16 aa, synthetic construct
  MOD_RES 16: note = Dab (L-2,4 diaminobutyric acid)
YKANYSSGESEFEFLX

SEQ ID NO: 146 - protein, 16 aa, synthetic construct
  REGION 1..16: note = Synthetic
YKANYSSGESEFVFLQ

SEQ ID NO: 147 - protein, 15 aa, synthetic construct
  MOD_RES 2: note = Dab (L-2,4 diaminobutyric acid)
QXYPHTHLEHQANPR

SEQ ID NO: 148 - protein, 11 aa, synthetic construct
  REGION 1..11: note = Synthetic
QKYPHTHLVHQ

SEQ ID NO: 149 - protein, 11 aa, synthetic construct
  REGION 1..11: note = Synthetic
HTHLVHQANPR

SEQ ID NO: 150 - protein, 16 aa, synthetic construct
  REGION 1..16: note = Synthetic
QKYPHTHLCHQANPRC

SEQ ID NO: 151 - protein, 21 aa, synthetic construct
  REGION 1..21: note = Synthetic
YKANYSSGESEFVFLQKYPHT

SEQ ID NO: 152 - protein, 20 aa, synthetic construct
  REGION 1..20: note = Synthetic
YSSGESEFVFLQKYPHTHLV

SEQ ID NO: 153 - protein, 21 aa, synthetic construct
  MOD_RES 16: note = Dab (L-2,4 diaminobutyric acid)
YKANYSSGESEFEFLXKYPHT

SEQ ID NO: 154 - protein, 21 aa, synthetic construct
  MOD_RES 12: note = Dab (L-2,4 diaminobutyric acid)
  MOD_RES 21: note = Orn (L-Ornithine)
YSSGESEFEFLXKYEHTHLVX

SEQ ID NO: 155 - protein, 13 aa, synthetic construct
  REGION 1..13: note = Synthetic
LLPKAPPLRELID

SEQ ID NO: 156 - protein, 9 aa, synthetic construct
  MOD_RES 2: note = Dab (L-2,4 diaminobutyric acid)
QXYPHTHLE

SEQ ID NO: 157 - protein, 10 aa, synthetic construct
  REGION 1..9: note = Synthetic
  MOD_RES 2: note = Dab (L-2,4 diaminobutyric acid)
QXYPHTHLEH

SEQ ID NO: 158 - protein, 12 aa, synthetic construct
  REGION 1..12: note = Synthetic
LCELICQYDVQR

SEQ ID NO: 159 - protein, 17 aa, synthetic construct
  REGION 1..17: note = Synthetic
VQRDDSSDGSLEDDDYH

SEQ ID NO: 160 - protein, 12 aa, synthetic construct
  REGION 1..12: note = Synthetic
IITMPTESDFLM

SEQ ID NO: 161 - protein, 12 aa, synthetic construct
  REGION 1..12: note = Synthetic
LNPFLEVKVTDT

SEQ ID NO: 162 - protein, 13 aa, synthetic construct
  REGION 1..13: note = Synthetic
VTDTPKSRRDFGL

SEQ ID NO: 163 - protein, 11 aa, synthetic construct
  REGION 1..11: note = Synthetic
FLEVKVTDTPK

SEQ ID NO: 164 - protein, 13 aa, synthetic construct
  REGION 1..13: note = Synthetic
LAVTFPGPGEDGL

SEQ ID NO: 165 - protein, 65 aa, synthetic construct
  REGION 1..65: note = Synthetic
  MOD_RES 1: note = acetylated on its terminal NH2
  MOD_RES 21: note = polyethylene glycol spacer AeeA (i.e., 8-amino-3,6-dioxaoctanoic acid or 2-[2-(2-aminoethoxy)ethoxy]acetic acid)
  MOD_RES 22: note = acetylation
  MOD_RES 41: note = polyethylene glycol spacer AeeA (i.e., 8-amino-3,6-dioxaoctanoic acid or 2-[2-(2-aminoethoxy)ethoxy]acetic acid)
STVINHYRMRGHSPFANLKSSQKYPHTHLVHQANPRGSAGPKKKKKISITEIKGVIVHRIETILF

SEQ ID NO: 166 - protein, 13 aa, synthetic construct
  REGION 1..13: note = Synthetic
TVFVQILRLIKPM

SEQ ID NO: 167 - protein, 39 aa, synthetic construct
  REGION 1..38: note = Synthetic
  MOD_RES 16: note = Dab (L-2,4 diaminobutyric acid)
  MOD_RES 20: note = epsilon lysine
YKANYSSGESEFEFLXKKKKISITEIKGVIVHRIETILF

SEQ ID NO: 168 - protein, 39 aa, synthetic construct
  REGION 1..39: note = Synthetic
  MOD_RES 20: note = epsilon lysine
YKANYSSGESEFVFLQKKKKISITEIKGVIVHRIETILF

SEQ ID NO: 169 - protein, 38 aa, synthetic construct
  MOD_RES 2: note = Dab (L-2,4 diaminobutyric acid)
  MOD_RES 19: note = epsilon lysine
QXYPHTHLEHQANPRKKKKISITEIKGVIVHRIETILF

SEQ ID NO: 170 - protein, 34 aa, synthetic construct
  REGION 1..34: note = Synthetic
  MOD_RES 15: note = epsilon lysine
QKYPHTHLVHQKKKKISITEIKGVIVHRIETILF

SEQ ID NO: 171 - protein, 34 aa, synthetic construct
  REGION 1..34: note = Synthetic
  MOD_RES 15: note = epsilon lysine
HTHLVHQANPRKKKKISITEIKGVIVHRIETILF

SEQ ID NO: 172 - protein, 39 aa, synthetic construct
  REGION 1..39: note = Synthetic
  MOD_RES 20: note = epsilon lysine
QKYPHTHLCHQANPRCKKKKISITEIKGVIVHRIETILF

SEQ ID NO: 173 - protein, 44 aa, synthetic construct
  REGION 1..44: note = Synthetic
  MOD_RES 25: note = epsilon lysine
YKANYSSGESEFVFLQKYPHTKKKKISITEIKGVIVHRIETILF

SEQ ID NO: 174 - protein, 43 aa, synthetic construct
  REGION 1..43: note = Synthetic
  MOD_RES 24: note = epsilon lysine
YSSGESEFVFLQKYPHTHLVKKKKISITEIKGVIVHRIETILF

SEQ ID NO: 175 - protein, 44 aa, synthetic construct
  REGION 1..43: note = Synthetic
  MOD_RES 16: note = Dab (L-2,4 diaminobutyric acid)
  MOD_RES 25: note = epsilon lysine
YKANYSSGESEFEFLXKYPHTKKKKISITEIKGVIVHRIETILF

SEQ ID NO: 176 - protein, 44 aa, synthetic construct
  MOD_RES 12: note = Dab (L-2,4 diaminobutyric acid)
  MOD_RES 21: note = Orn (L-Ornithine)
  MOD_RES 25: note = epsilon lysine
YSSGESEFEFLXKYEHTHLVXKKKKISITEIKGVIVHRIETILF

SEQ ID NO: 177 - protein, 36 aa, synthetic construct
  REGION 1..36: note = Synthetic
  MOD_RES 17: note = epsilon lysine
LLPKAPPLRELIDKKKKISITEIKGVIVHRIETILF

SEQ ID NO: 178 - protein, 32 aa, synthetic construct
  MOD_RES 2: note = Dab (L-2,4 diaminobutyric acid)
  MOD_RES 13: note = epsilon lysine
QXYPHTHLEKKKKISITEIKGVIVHRIETILF

SEQ ID NO: 179 - protein, 32 aa, synthetic construct
  MOD_RES 2: note = Dab (L-2,4 diaminobutyric acid)
  MOD_RES 14: note = epsilon lysine
QXYPHTHLEHKKKKKKKIITITRIITIITTID

SEQ ID NO: 180 - protein, 35 aa, synthetic construct
  REGION 1..35: note = Synthetic
  MOD_RES 16: note = epsilon lysine
LCELICQYDVQRKKKKISITEIKGVIVHRIETILF

SEQ ID NO: 181 - protein, 40 aa, synthetic construct
  REGION 1..40: note = Synthetic
  MOD_RES 21: note = epsilon lysine
VQRDDSSDGSLEDDDYHKKKKISITEIKGVIVHRIETILF

SEQ ID NO: 182 - protein, 35 aa, synthetic construct
  REGION 1..35: note = Synthetic
  MOD_RES 16: note = epsilon lysine
IITMPTESDFLMKKKKISITEIKGVIVHRIETILF

SEQ ID NO: 183 - protein, 35 aa, synthetic construct
  REGION 1..35: note = Synthetic
  MOD_RES 16: note = epsilon lysine
LNPFLEVKVTDTKKKKISITEIKGVIVHRIETILF

SEQ ID NO: 184 - protein, 36 aa, synthetic construct
  REGION 1..36: note = Synthetic
  MOD_RES 17: note = epsilon lysine
VTDTPKSRRDFGLKKKKISITEIKGVIVHRIETILF

SEQ ID NO: 185 - protein, 34 aa, synthetic construct
  REGION 1..34: note = Synthetic
  MOD_RES 15: note = epsilon lysine
FLEVKVTDTPKKKKKISITEIKGVIVHRIETILF

SEQ ID NO: 186 - protein, 36 aa, synthetic construct
  REGION 1..36: note = Synthetic
  MOD_RES 17: note = epsilon lysine
LAVTFPGPGEDGLKKKKISITEIKGVIVHRIETILF

SEQ ID NO: 187 - protein, 10 aa, synthetic construct
  REGION 1..10: note = Synthetic
YRMRGHSPFA

SEQ ID NO: 188 - protein, 36 aa, synthetic construct
  REGION 1..36: note = Synthetic
  MOD_RES 17: note = epsilon lysine
TVFVQILRLIKPMKKKKISITEIKGVIVHRIETILF

SEQ ID NO: 189 - protein, 12 aa, synthetic construct
  REGION 1..12: note = Synthetic
HYRMRGHSPFAN

SEQ ID NO: 190 - protein, 33 aa, synthetic construct
  REGION 1..33: note = Synthetic
  MOD_RES 14: note = epsilon lysine
YRMRGHSPFAKKKKISITEIKGVIVHRIETILF

SEQ ID NO: 191 - protein, 35 aa, synthetic construct
  REGION 1..35: note = Synthetic
  MOD_RES 16: note = epsilon lysine
HYRMRGHSPFANKKKKISITEIKGVIVHRIETILF

SEQ ID NO: 192 - protein, 4 aa, synthetic construct
  REGION 1..4: note = Synthetic
  MOD_RES 4: note = epsilon lysine
KKKK

SEQ ID NO: 193 - protein, 19 aa, synthetic construct
  REGION 1..19: note = Synthetic
  VARIANT 4: note = X is S or T
  VARIANT 7: note = X is K or R
  VARIANT 8: note = X is G or T
  VARIANT 12: note = X is H or T
  VARIANT 13: note = X is K or R
ISIXEIXXVIVXXIETILF